(19) United States
(12) Patent Application Publication
(10) Pub. No.: US 2003/0162221 A1
(43) Pub. Date: Aug. 28, 2003

(54) Yeast proteome analysis

(76) Inventors: Gary Bader, North York (CA); Shane Climie, Toronto (CA); Daniel Durocher, Toronto (CA); Joseph Michael Daniel Figeys, Pickering (CA); Albrecht Gruhler, Odense N (DK); Adrian Mark Heilbut, Toronto (CA); Yuen Ho, Toronto (CA); Lynda A. Moore, Toronto (CA); Michael Moran, Toronto (CA); Brenda Muskat, Toronto (CA); Michael Tyers, Toronto (CA); Cheryl Deanna Wolting, Toronto (CA)

Correspondence Address: ROPES & GRAY, ONE INTERNATIONAL PLACE, BOSTON, MA, 02110-2624, US

(21) Appl. No.: 10/252,749

(22) Filed: Sep. 23, 2002

Related U.S. Application Data
(60) Provisional application No. 60/323,930, filed on Sep. 21, 2001.
(60) Provisional application No. 60/341,213, filed on Oct. 30, 2001.
(60) Provisional application No. 60/345,286, filed on Jan. 4, 2002.

(51) Int. Cl. G01N 33/53; G06F 19/00; G01N 33/48; G01N 33/50

(52) U.S. Cl. 435/7.1; 702/19

(57) ABSTRACT

Methods and reagents for high throughput analysis of protein-protein interaction networks using mass spectrometry.

### RELATED APPLICATIONS

This application claims priority to U.S. Provisional Applications, 60/323,930, filed on Sept. 21, 2001; 60/341,213, filed on Oct. 30, 2001; and 60/345,286, filed on Jan. 4, 2002, the entire content of which are incorporated by reference herein.

### FIELD OF THE INVENTION

The invention relates to high-throughput proteome analysis.

### BACKGROUND OF THE INVENTION

Cellular behavior is determined by the dynamic interactions of a vast array of proteins that form complexes and higher order networks¹. The global coordination of cellular function is presumed to require the concerted regulation of such networks. As the human genome is predicted to contain more than 30,000 discrete open reading frames, which may each give rise to multiple protein variants via splicing and other modifications, the problem of systematically decoding protein interactions is daunting. To date, attempts to generate comprehensive protein-protein interaction maps have relied on the yeast two-hybrid system, whereby binary interactions are detected via bridging of transcription factor DNA binding and transactivation domains, thereby activating reporter gene expression². Large scale applications of the two-hybrid method have yielded numerous relevant protein-protein interactions³⁻⁵. In a more direct approach, protein complexes can be purified from cell lysates followed by identification of each constituent. With the advent of ultra-sensitive mass spectrometric protein identification methods, it has become feasible to consider such an approach on a proteome-wide scale⁶⁻⁸.

### SUMMARY OF THE INVENTION

The instant invention is related to the high-throughput (HTP) analysis of protein interaction networks by highly sensitive mass spectrometric identification methods (HTP-MS/MS), also known as high throughput MS/MS protein complex identification (HMS-PCI).

One aspect of the invention provides a method of identifying a protein interaction network using high throughput tandem mass spectrometry, particularly in the setting of proteome-wide analysis. Typically, a bait protein (either in its native form or a modified form—such as an epitope tagged form) is used to retrieve binding prey proteins from an environment, preferably a native environment inside a cell, and complexes comprising the bait and prey proteins are separated and subjected to mass spectrometry analysis to identify prey proteins.

Thus in one aspect, the invention provides a method for identifying a protein interaction network comprising two or more bait proteins, comprising: (a) isolating complexes comprising at least one of said two or more bait proteins and their prey proteins from a sample; (b) separating said complexes; and (c) determining the identity of the prey proteins in each of said complexes using mass spectrometry, thereby identifying the protein interaction network.

In another aspect, the invention provides a method for identifying a protein interaction network comprising two or more bait proteins, comprising: (a) contacting said two or more bait proteins with a sample containing potential prey proteins, wherein the bait proteins and complexes comprising at least one said bait protein(s) are capable of being separated from other proteins in the sample; (b) separating said complexes comprising at least one said bait proteins and their prey proteins; (c) identifying prey proteins in the complexes using mass spectrometry, thereby identifying the protein interaction network.

In one embodiment, the protein interaction network comprises 5, 10, 20, 50, 100, 200 or more bait proteins. In a related embodiment, the protein interaction network comprises 2%, 5%, 10%, 20%, 30%, 40%, 50%, 75%, 90%, or 100% of the proteome of a given genome. In a preferred embodiment, the proteome is a yeast (such as S. cerevisiae or S. pombe) proteome.

In another embodiment, the protein interaction network comprises all bait proteins known to be involved in the same biochemical pathway or biological process.

In another embodiment, the protein interaction network comprises the same type of proteins, for example, protein kinases, protein phosphatases, receptors, G proteins, ion channels, transcription factors, etc.

In one embodiment, a bait protein or protein of interest used in a method of the invention is unmodified. In another embodiment, a bait protein or protein of interest is synthesized as a fusion protein with a heterologous polypeptide to facilitate its retrieval from said biological sample. Examples of the heterologous polypeptides include: GST, HA epitope, c-myc epitope, 6-His tag, FLAG tag, biotin, or MBP. Bait proteins can be expressed in a host cell as an exogenous polypeptide.

A bait protein may be immobilized to facilitate isolation of the complexes. For example, a bait protein may be directly or indirectly (e.g. with an antibody specific for the epitope tag) bound to a suitable carrier or solid support such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc.

In a preferred embodiment, the sample is a biological sample, preferably an extract of a cell. In one embodiment, the extract is concentrated. The cell can be a yeast cell, or it can be a higher eukaryotic cell, such as a nematode (C. elegans), insect, fish, reptile, amphibian, plant, or mammalian cell, or more preferably, a human cell.

In one embodiment of the invention, complex formation between bait and prey proteins is induced using an extracellular or intracellular factor.

In one embodiment, complexes comprising at least one bait protein and its prey proteins are isolated by immunoprecipitation. In a related embodiment, complexes are isolated by a GST pull-down assay.

In one embodiment, complexes are digested by protease before separation. The digestion can be performed on either purified protein or on protein samples in gel.

In one embodiment, complexes are separated by SDS-PAGE. In a related embodiment, complexes are separated by chromatography, such as HPLC, or any other suitable protein separation means commonly known in the art, including chromatography, HPLC, Capillary Electrophoresis (CE), isoelectric focusing (IEF).

In a particular embodiment, complexes are separated by SDS-PAGE, and digested by in-gel protease digestion.

In an aspect, the mass spectrometry employed in a method of the invention is tandem mass spectrometry (MS/MS). In a preferred embodiment, the MS/MS is coupled with Liquid Chromatography (LC).

In another embodiment, protein sequences obtained from tandem mass spectrometry are compared against protein sequence databases in order to determine the identity of the proteins. In a preferred embodiment, said protein sequence databases include a combination of public database and proprietary database. For example, computer programs including but not limited to the following may be used: TBLASTN, BLASTP, FASTA, TFASTA, and CLUSTALW (Altschul et al., 1990, J. Mol. Biol. 215(3):403-10; see, Pearson and Lipman, 1988, Proc. Natl. Acad. Sci. USA 85(8):2444-8; Thompson, et al., 1994, Nucleic Acids Res. 22(22):4673-80; Higgins, et al., 1996, Methods Enzymol 266:383-402).

In another embodiment, the method further comprises repeating steps (a)-(c) using prey proteins identified from previous round(s) as new bait proteins, wherein said new bait proteins are different from any bait proteins used in said previous round.

The invention also provides libraries of information on a protein interaction network identified using a method of the invention, methods to construct such libraries, and data sharing systems which enable efficient utilization of such libraries. Furthermore, the invention provides databases which accommodate and maintain libraries of information relative to such protein interaction network, methods and systems to construct such databases, methods and systems to enable a user/client to search through such databases for desired information, methods and systems to transmit to a client desired pieces of information concerning protein interaction networks that are housed in databases, tangible electronic means to record and make use of such systems and databases, and apparatus to enable construction and search of databases and/or transmission of desired information to a client. Detailed methods of creating databases as described herein and search engines for these databases, based on information obtained using a suitable method of the invention, are well-known in the art, and thus will not be described in detail.

Therefore, in one aspect, the invention provides a database of protein interaction network(s) identified by a method of the instant invention, comprising information regarding two or more bait proteins and their interactions.

In one embodiment, the information includes: the identity of all bait proteins and their interacting prey proteins, the conditions under which the interactions are observed and/or the identity of the sample from which said information is obtained.

In one embodiment, one or more filters are used to modify the protein interaction network database.

In one embodiment, the database is verified by information obtained from a public or proprietary database.

In one embodiment, the database comprises a set of potential protein interactions and molecular complexes in a given proteome, under one or more specific conditions. In a related embodiment, the database comprises at least about 20%, 30%, 40%, 50%, 60%, 70%, 80%, 90% or 95% of the potential protein interactions of a given organism The database can also include annotations of certain protein-protein interaction information obtained from searching available scientific literature using proprietary software. Such annotations can be dynamically updated, preferably automatically, by repeated searches performed at predetermined time intervals.

In one embodiment, the database comprises a set of protein interactions, preferably a set of at least about 20%, 30%, 40%, 50%, 60%, 70%, 80%, 90% or 95% of the protein interactions, in a yeast cell. In a related embodiment, the database comprises all homologous proteins related to any given set of yeast protein interactions. “Homologous” as used herein means any protein that is at least 75%, preferably 80%, 85%, 90%, or most preferably 95%, even 99% identical to a given protein. Usually, a homologous protein exists in a different species, such as in a worm, insect, plant, or mammal, most preferably in human.

In one aspect of the invention, a database is provided comprising a yeast protein interaction network. In a particular embodiment, the database comprises a set of more than 4000 yeast protein interactions. In another particular embodiment, the database comprises about 20-30%, preferably about 25-30%, more preferably about 29% of the yeast proteome. In a preferred embodiment, the database comprises the complexes of Table 2, 4A, 4B, 5A, 5B, and 7.

Another aspect of the invention provides a method of identifying differences in protein interaction networks comprising one or more selected bait proteins, comprising:

(a) providing a first protein interaction network identified by (i) isolating complexes comprising a selected bait protein(s) and prey proteins from a first sample; (ii) separating complexes comprising the bait protein(s) and prey proteins; and (iii) determining the identity of the prey proteins, preferably by mass spectrometry, thereby identifying the first protein interaction network;

(b) providing a second protein interaction network identified by (i) isolating complexes comprising the selected bait protein(s) and prey proteins from a second sample; (ii) separating complexes comprising the bait protein(s) and prey proteins; and (iii) determining the identity of the prey proteins, preferably by mass spectrometry, thereby identifying the second protein interaction network; and

(c) comparing the first and second protein interaction networks, thereby identifying differences in the protein interaction networks.

In one embodiment, the first sample is from a tumor tissue, and the second sample is from a normal tissue of the same tissue type. In another embodiment, the tumor tissue and the normal tissue are from the same patient. In another embodiment, the first sample and the second sample are from different developmental stages of the same organism. In another embodiment, the first sample is from a tissue, and the second sample is from the same tissue type after a treatment. Such tissue can be, for example, a tumor tissue. Such treatment can be, for example, chemotherapy or radiotherapy.

The invention also provides methods for assaying for changes in protein interaction networks in response to intracellular or extracellular factors.

Therefore, a method is provided for assaying for changes in protein interaction networks in response to an intracellular or extracellular factor comprising: (a) contacting two or more bait proteins with a sample containing prey proteins in the presence of an intracellular or extracellular factor, wherein the bait proteins and complexes comprising the bait proteins are capable of being separated from other proteins in the sample; (b) separating complexes comprising bait proteins and prey proteins; (c) identifying prey proteins in the complexes using mass spectrometry, thereby identifying the protein interaction network; and (d) comparing the protein interaction network identified in (c) with a protein interaction network identified in the absence of the intracellular or extracellular factor.

Another aspect of the invention provides a method to identify potential protein targets for drug design and pharmaceutical research, comprising identifying a network of protein interactions comprising a protein of interest, such as a previously known drug target, using the method or database of the instant invention, thereby identifying other related drug targets for a given biological process.

Thus, in this respect, the invention provides a method of conducting a pharmaceutical business, comprising: (a) identifying a protein interaction network of one or more known bait protein from a sample using a method of the invention wherein said bait protein is a potential drug target; (b) identifying, among prey proteins that interact with said bait protein in the protein interaction network, new potential drug targets; (c) licensing, to a third party, the rights for further drug development of inhibitors or activators of the drug target.

In a related aspect, the invention provides a method of conducting a pharmaceutical business, comprising: (a) identifying a protein interaction network of one or more known bait proteins from a biological sample using a method of the invention, wherein said bait protein is a potential drug target; (b) identifying, among prey proteins that interact with said bait proteins in the protein interaction network, new potential drug targets; (c) identifying compounds that modulate activity of said new potential drug targets; (d) conducting therapeutic profiling of compounds identified in step (c), or further analogs thereof, for efficacy and toxicity in animals; and, (e) formulating a pharmaceutical preparation including one or more compounds identified in step (d) as having an acceptable therapeutic profile.

In one embodiment, the method further comprises an additional step of establishing a distribution system for distributing the pharmaceutical preparation for sale. In a related embodiment, the method further comprises establishing a sales group for marketing the pharmaceutical preparation.

Methods and reagents provided by the instant invention are useful for rapid, efficient identification of protein-protein interactions in a large scale. In one respect, it provides a platform for doing drug screen related pharmaceutical research in a genetically well defined system such as yeast, by virtue of sequence homology between yeast and its higher eukaryotic counterparts such as human. In another respect, it also offers a high throughput means to study protein-protein interaction and signaling networks directly in higher organisms. The ultimate utility of any large scale platform rests upon its ability to reliably glean new insights into biological function. By the criterion of extensive literature validation, initial study demonstrates that the HTP-MS/MS approach is well suited to this task Given that the encoded set of human proteins is nominally 5-fold greater than the set of predicted yeast proteins, comprehensive analysis of the human proteome is feasible with current HTP-MS/MS platforms.

The methods of the present invention, as described above, may be practiced using kits for identifying protein interaction networks comprising two or more bait proteins. A kit will generally include expressable recombinant vectors for generating bait proteins.

The invention also provides a method for constructing a protein interaction network map for a proteome comprising: (a) identifying a protein interaction network using a method of the invention, and (b) displaying the network as a linkage map.

The invention also provides an integrated modular system for performing methods of the invention. In an embodiment, the system comprises one or more of the following modules: (a) a module for retrieving recombinant clones encoding bait proteins; (b) an automated immunoprecipitation module for purification of complexes comprising bait and prey proteins; (c) an analysis module for further purifying the proteins from (b) or preparing fragments of such proteins that are suitable for mass spectrometry; (d) a mass spectrometer module for automated analysis of fragments from (c); (d) a computer module comprising an integration software for communication among the modules of the system and integrating operations; and (e) a module for performing an automated method of the invention.

The integrated modular system may be automated for high throughput operation.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

### DESCRIPTION OF DRAWINGS

The invention will be better understood with reference to the drawings in which:

FIG. 1 illustrates a HMS-PCI strategy a, Flow diagram of approach b, Protein complexes captured onto anti-FLAG agarose resin, eluted and resolved by SDS-PAGE c, Proteins specific to the elution are excised, digested with trypsin and subject to LC-MS/MS. Matches of fragmentation spectra to databases unambiguously identify proteins in the sample, as shown here for Ste12.

FIG. 2 illustrates kinase-based signaling networks a, The mating pheromone MAPK pathway. The core Ste11-Ste7-Fus3-Kss1 MAPK module phosphorylates downstream transcription factors and other targets. Blue indicates proteins identified in association with Kss1 b, Interaction diagram for Kss1 complexes c, Interaction diagram for Cdc28 complexes. Arrows point from the bait protein to the interaction partner. Black arrows indicate known interactions; red arrows indicate novel interactions.

FIG. 3 illustrates the DNA damage response network. Interactions were initially nucleated from 86 proteins implicated in the DDR. Blue nodes indicate known interactions within dedicated complexes as labeled. Black arrows indicate known interactions; red arrows indicate novel interactions.

FIG. 4 shows a graphical representation of large-scale protein interaction networks and comparison to literature interactions a, entire HMS-PCI network in spoke model representation b, overlap of spoke model and PreBIND c, overlap of HTP-Y2H dataset³ and PreBIND d, overlap of spoke model and HTP-Y2H dataset³. Blue nodes and edges are literature-derived interactions; red nodes and edges are novel interactions detected by HTP approaches. For clarity, simple binary interactions are not shown in panels b (36 interactions removed), c (20 interactions removed) and d (30 interactions removed).

FIG. 5 shows the percentage of total baits bound per each interacting protein. Each interacting protein was plotted versus the percentage of the total baits it bound. To the left of the dotted line, the percentage of total baits bound increases dramatically. This corresponds to 3% of total baits bound, and was taken as the percentage of baits bound that at and above which the interacting protein is likely a background, promiscuous binder.

### DETAILED DESCRIPTION OF THE INVENTION

Definitions

“Binding,” “bind” or “bound” refers to an association, which may be a stable association, between two molecules, e.g., between a protein ligand and a another polypeptide, due to, for example, electrostatic, hydrophobic, ionic and/or hydrogen-bond interactions under physiological conditions.

“Bait” or “bait protein” refers to proteins used in an assay aimed at identifying interacting or “prey” proteins to preferably define a protein interaction network. A bait protein may comprise all or part of a target molecule which has been implicated in a biological process of interest, or for which the function is sought. A bait protein may include functional domains of a wide variety of proteins including receptors, ligands, enzymes, transcription proteins, cell cycle proteins, etc. In an aspect of the invention, bait proteins are selected from a proteome (e.g. yeast) including but not limited to yeast proteins implicated in DNA damage and repair, protein kinases, protein phosphatases, receptors, G proteins, ion channels, and transcription factors.

A bait protein may be in its native form, or may be modified to facilitate the identification process. For example, the bait protein may be synthesized as a fusion protein so that it contains a heterologous domain/motif that is useful for isolating the fusion protein. Any known or commonly used polypeptides for which an isolation method is available can be utilized as the heterologous domain in the bait fusion protein. Such heterologous domains may include (but are not limited to) GST, an epitope tag (FLAG tag, c-myc tag, HA (human Influenza virus hemagglutinin) tag, or other commonly used or commercially available epitope tags, etc.), 6-His tag, biotin, GFP (green fluorescent protein), MBP (Maltose Binding Protein), etc. An advantage of using the fusion bait protein is that the need to prepare an antibody for each potential bait protein is obviated, and relatively uniform efficiency of retrieving complexes containing the bait proteins can be achieved. Also, the fusion protein may be easily differentiated from the endogenous proteins, which may or may not be expressed in a given cell at a given time.

“Prey” or “prey protein” refers to any polypeptide that binds to a “bait” protein, either directly by binding to the bait protein, or indirectly by binding to other proteins so that the bait and the prey exist in the same multi-polypeptide complex, under a given condition, including a native or physiological condition or an experimental condition.

“Complex” generally refers to an association between at least two moieties (e.g. chemical or biochemical) that have an affinity for one another. Examples of complexes include associations between antigen/antibodies, lectin/avidin, antibody/anti-antibody, receptor/ligand, enzyme/ligand and the like. “Member of a complex” refers to one moiety of the complex, such as an antigen or ligand, or a bait and a prey. “Protein complex” or “polypeptide complex” refers to a complex comprising at least one polypeptide. In the context of the present invention, a complex includes a prey protein bound to a bait protein.

“Exogenous” means caused by factors or an agent from outside the organism or system, or introduced from outside the organism or system, specifically: not normally synthesized within the organism or system. A fusion/tagged protein expressed from an introduced plasmid may be considered exogenous to the host cell expressing the fusion protein, although the host itself may express an endogenous version of the same protein.

“Extracellular factor” includes a molecule or a change in the environment that is transduced intracellularly via cell surface proteins (e.g. cell surface receptors) that interact, directly or indirectly, with a signal. An extracellular factor includes any compound or substance that in some manner specifically alters the activity of a cell surface protein. Examples of such signals or factors include, but are not limited to growth factors, that bind to cell surfaces and/or intracellular receptors and ion channels and modulate the activity of such receptors and channels. The signals and factors include analogs, derivatives, mutants, and modulators of such growth factors.

“Intracellular factor” includes a molecule or a change in the cell environment that is transduced in the cell via cytoplasmic proteins that interact, directly or indirectly with a signal. An intracellular factor includes any compound or substance that in some manner specifically alters the activity of a cytoplasmic protein involved in a biological or signal transduction pathway.

“Filter” when referring to data processing means eliminating certain obtained/observed data based on certain preset criteria For example, a protein sample loaded onto one lane of a SDS-PAGE gel may occasionally spill-over the adjacent lanes, which may be subsequently detected by the highly sensitive MS/MS analysis. Thus, a protein that is the same as a bait protein on gel loaded within 3 gel lanes on either side of the bait protein on a gel may be designated as a “spillover,” and filtered from the data set. More than one filter set can be used to modify the final protein interaction network.

“GST pull-down assay” refers to a method comprising incubating GST-fusion proteins within a sample (such as cell lysate) with GST-binding moieties, typically glutathione beads, and “pulling-down,” proteins binding to the GST-fusion protein. The process is analogous to immunoprecipitation using antibodies against specific proteins.

“High throughput” refers to the ability to process large amount of samples in a given process, method, or assay, etc. In a preferred embodiment, the high throughput process is conducted with an automated machine(s), which is optionally controlled by computer software or human or both.

“Hit” generally refers to a desired result in an assay. For example, in an assay searching for interacting proteins of a given “bait” protein, a hit refers to a “prey” protein that is identified by the assay/process as being able to interacting with the bait protein.

“Molecular complex” refers to assemblages composed of more than two polypeptides. Each component of the molecular complex binds together by non-covalent bonds. There is no limitation on the number of proteins of the complex. Preferably, a molecular complex comprises two, three, four, five, six, seven, eight, nine, ten, fifteen, twenty, twenty-five, or thirty interacting proteins that potentially have a common origin, function, structure, mechanism, or activity.

“Analyzing a protein by mass spectrometry” or similar wording refers to using mass spectrometry to generate information which may be used to identify or aid in identifying a protein. Such information includes, for example, the mass or molecular weight of a protein, the amino acid sequence of a protein or protein fragment, a peptide map of a protein, and the purity or quantity of a protein.

“Protein interaction network” refers to a collection of information regarding protein-protein interactions among certain proteins. A protein interaction network may contain a number of bait proteins, as well as prey proteins identified as being able to directly or indirectly bind with these bait proteins. A given protein interaction network may be verified and/or expanded by including some of the initially identified prey proteins as bait proteins for subsequent rounds of assays aimed at identifying more interaction proteins. The protein interaction network may be represented using a number of models, for example, see the spoke model and the matrix model described below. A protein interaction network may also be associated with a given condition (cell type, developmental stage, cell-cycle stage, complex isolation condition, etc.) when necessary, since the same set of bait proteins may yield different protein interaction networks under different conditions. Thus a protein interaction network may represent all possible interactions among conditions, or represent interactions observed in a specific condition. A protein interaction network may represent the entire interaction map of a proteome that specifies the entire signal transduction and metabolic networks of a cell such as a yeast cell.

A protein interaction network typically comprises two or more proteins. In certain protein interaction networks, any two proteins within the network are directly or indirectly connected. In the latter case, if protein A and X are indirectly connected, it includes the situation that protein A binds protein B, and protein X binds protein Y, wherein A and X do not directly interact with each other, but B and Y directly interact with each other, although the A-B, B-Y, and X-Y interactions need not occur under the same condition or in the same sample. It also includes the situation wherein B and Y are indirectly connected via other proteins. This is analogous to the internet wherein any two computers on the internet can be directly or indirectly connected. In certain other protein interaction networks, at least two proteins are not connected to each other, either directly or indirectly. This is analogous to two or more separate local area networks wherein each member of a local area network is only directly or indirectly connected with other members of the same network, but not members belonging to other local area networks.

“Promiscuous binder” refers to proteins that bind to numerous bait proteins, and which are excluded from a protein interaction network data set.

“Proteome” refers to all the proteins that can be encoded by a given genome, which is in turn all the genetic material (including all the genes) of a given organism. Not all proteins within a given proteome are necessarily expressed at the same time, in the same cell type/tissue origin. Due to changes in conditions such as developmental, environmental, physiological, or pathological conditions, any given tissue/cell type may only express a fraction of the total number of proteins that can be encoded by a given genome (or, a fraction of the total proteome). “Troteome” may also refer to the entire complement of proteins expressed by a given tissue or cell type.

“Solid support” or “carrier,” used interchangeably, refers to a material which is an insoluble matrix, and may (optionally) have a rigid or semi-rigid surface. Such materials may take the form of small beads, pellets, disks, chips, dishes, multi-well plates, wafers or the like, although other forms may be used. In some embodiments, at least one surface of the substrate will be substantially flat.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the—sequences of the present invention.

The term “percent identical” refers to sequence identity between two amino acid sequences or between two nucleotide sequences. Identity can each be determined by comparing a position in each sequence which may be aligned for purposes of comparison When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both polypeptide and DNA databases.

Databases with individual sequences are described in Methods in Enzymology, ed. Doolittle, supra. Some exemplary public databases include GenBank, EMBL, DNA Database of Japan (DDBJ), SwissProt, PIR and other databases derived therefrom. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489. Alternatively, certain commercial software packages such as LaserGene from DNAStar inc. can be used for certain aspects of sequence analysis. Multiple softwares and databases may be used in any analysis.

The terms “protein”, “polypeptide” and “peptide” are used interchangeably herein when referring to a natural or recombinant gene product of fragment thereof.

The term “recombinant protein” refers to a polypeptide of the present invention which is produced by recombinant DNA techniques, wherein generally, DNA encoding a polypeptide is inserted into a suitable expression vector which is in turn used to transform a host cell to produce the heterologous polypeptide. Moreover, the phrase “derived from”, with respect to a recombinant gene, is meant to include within the meaning of “recombinant protein” those polypeptides having an amino acid sequence of a native polypeptide, or an amino acid sequence similar thereto which is generated by mutations including substitutions and deletions (including truncation) of a naturally occurring form of the polypeptide.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject target gene polypeptides. For example, excision of a target sequence which interferes with the expression of a recombinant target gene, such as one which encodes an antagonistic homolog or an antisense transcript, can be designed to activate expression of that gene. This interference with expression of the polypeptide can result from a variety of mechanisms, such as spatial separation of the target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allows for promoter driven transcriptional activation.

“Phospho-protein” is meant a polypeptide that can be potentially phosphorylated on at least one residue, which can be either tyrosine or serine or threonine or any combination of the three. Phosphorylation can occur constitutively or be induced.

“Post-translational modification” is meant any changes/modifications that can be made to the native polypeptide sequence after its initial translation. It includes, but are not limited to, phosphorylation/dephosphorylation, prenylation, myristoylation, palmitoylation, limited digestion, irreversible conformation change, methylation, acetylation, modification to amino acid side chains or the amino terminus, and changes in oxidation, disulfide-bond formation, etc.

“Sample” as used herein generally refers to a type of source or a state of a source, for example, a given cell type or tissue. The state of a source may be modified by certain treatments, such as by contacting the source with a chemical compound, before the source is used in the methods of the invention. It should be noted that protein interaction network data based on “a sample” does not necessarily comprise results obtained from a single experiment. Rather, to completely determine a protein interaction network, multiple experiments are often needed, and the combined results of which are used to construct the protein interaction network data for that particular sample.

Methods of the Invention

A bait protein for use in the methods of the invention can be expressed in high levels in any given host cell using proper molecular biology techniques. A skilled artisan shall be able to determine the best suitable system including expression vectors, suitable host cells, means to introduce heterologous DNA into such host cells, optimal conditions for protein expression, etc. for any given protein. The example herein is provided for illustration purpose only and shall not be construed as a limitation of the scope of the invention in any way.

A typical vector suitable for host cell expression shall contain at least the necessary elements for transcription and translation of the target protein. To avoid potential toxicity of heterologous protein expression in the host cell, the expression can be under the control of an inducible promoter, such as a galactose-inducible promoter. The vector used can optionally contain an epitope tag against which an antibody, preferably a commercial antibody is available so that the synthesized fusion protein can be readily isolated using a standardized immunoprecipitation procedure.

To facilitate large scale high throughput experiments, the vector can be further adapted to be compatible with the Gateway™ system (Invitrogen) by including att sites so that batch cloning can be achieved using recombination-based cloning. PCR amplification can then be used to generate gene fragments flanked by att sites for efficient cloning into the Gateway vector. It should be noted that other similar systems of recombination-based cloning can also be used and are also within the scope of the instant invention.

Generally, any given protein of interest or bait protein can be expressed in a host cell, either with or without an epitope tag against which an antibody is available, and protein complexes encompassing this protein of interest are isolated using any of many suitable techniques such as immunoprecipitation. The isolated complexes can be separated on SDS-PAGE gel and each band representing at least one potentially interacting protein can be digested by protease such as trypsin or other equivalent enzymes that generates C-terminal basic amino acids such as Arg or Lys. The digested protein samples are then analyzed by tandem mass spectrometry (MS/MS) to obtain sequence information of at least a few peptide fragments. These data will then be compared with known sequences in the publicly available protein/polynucleotide database to unequivocally identify those interacting proteins.

One aspect of the instant invention discloses a method for large scale analysis of protein-protein interactions using ultra-sensitive mass spectrometry. The mass spectrometry platform is based on a high throughput LC-MS/MS approach for protein complex identification, which is referred to herein as HMS-PCI. This platform is much more powerful than commonly used MALDI-TOF platforms. Although MALDI-TOF is capable of high throughput, it does not readily allow for peptide fragmentation and is therefore limited to highly purified preparations from organisms with small genomes. In contrast, LC-MS/MS instrumentation allows identifications to be made from complex protein mixtures because peptide sequence information is obtained. A direct comparison between studies in yeast with a MALDI-TOF instrument and studies on the same samples shows that the LC-MS/MS approach yielded a much greater hit rate. It is worth noting that the HMS-PCI approach is well suited to analysis of complex proteomes (e.g., the human proteome), whereas MALDI-based platforms are not.

Mass Spectrometers, Detection Methods and Sequence Analysis

In certain embodiments, the interacting proteins are identified by protease digestion followed by mass spectrometry. During the past decade, new techniques in mass spectrometry have made it possible to accurately measure with high sensitivity the molecular weight of peptides and intact proteins. These techniques have made it much easier to obtain accurate peptide masses of a protein for use in databases searches. Mass spectrometry provides a method, of protein identification that is both very sensitive (10 fmol-1 pmol) and very rapid when used in conjunction with sequence databases. Advances in protein and DNA sequencing technology are resulting in an exponential increase in the number of protein sequences available in databases. As the size of DNA and protein sequence databases grows, protein identification by correlative peptide mass matching has become an increasingly powerful method to identify and characterize proteins.

Mass Spectrometry

Mass spectrometry, also called mass spectroscopy, is an instrumental approach that allows for the gas phase generation of ions as well as their separation and detection. The five basic parts of any mass spectrometer include: a vacuum system; a sample introduction device; an ionization source; a mass analyzer; and an ion detector. A mass spectrometer determines the molecular weight of chemical compounds by ionizing, separating, and measuring molecular ions according to their mass-to-charge ratio (m/z). The ions are generated in the ionization source by inducing either the loss or the gain of a charge (e.g. electron ejection, protonation, or deprotonation). Once the ions are formed in the gas phase they can be electrostatically directed into a mass analyzer, separated according to mass and finally detected. The result of ionization, ion separation, and detection is a mass spectrum that can provide molecular weight or even structural information.

A common requirement of all mass spectrometers is a vacuum. A vacuum is necessary to permit ions to reach the detector without colliding with other gaseous molecules. Such collisions would reduce the resolution and sensitivity of the instrument by increasing the kinetic energy distribution of the ion's inducing fragmentation, or preventing the ions from reaching the detector. In general, maintaining a high vacuum is crucial to obtaining high quality spectra.

The sample inlet is the interface between the sample and the mass spectrometer. One approach to introducing sample is by placing a sample on a probe which is then inserted, usually through a vacuum lock, into the ionization region of the mass spectrometer. The sample can then be heated to facilitate thermal desorption or undergo any number of high-energy desorption processes used to achieve vaporization and ionization.

Capillary infusion is often used in sample introduction because it can efficiently introduce small quantities of a sample into a mass spectrometer without destroying the vacuum. Capillary columns are routinely used to interface the ionization source of a mass spectrometer with other separation techniques including gas chromatography (GC) and liquid chromatography (LC). Gas chromatography and liquid chromatography can serve to separate a solution into its different components prior to mass analysis. Prior to the 1980's, interfacing liquid chromatography with the available ionization techniques was unsuitable because of the low sample concentrations and relatively high flow rates of liquid chromatography. However, new ionization techniques such as electrospray were developed that now allow LC/MS to be routinely performed. One variation of the technique is that high performance liquid chromatography (HPLC) can now be directly coupled to mass spectrometer for integrated sample separation/preparation and mass spectrometer analysis.

In terms of sample ionization, two of the most recent techniques developed in the mid 1980's have had a significant impact on the capabilities of Mass Spectrometry: Electrospray Ionization (ESI) and Matrix Assisted Laser Desorption/Ionization (MALDI). ESI is the production of highly charged droplets which are treated with dry gas or heat to facilitate evaporation leaving the ions in the gas phase. MALDI uses a laser to desorb sample molecules from a solid or liquid matrix containing a highly UV-absorbing substance.

The MALDI-MS technique is based on the discovery in the late 1980s that an analyte consisting of, for example, large nonvolatile molecules such as proteins, embedded in a solid or crystalline “matrix” of laser light-absorbing molecules can be desorbed by laser irradiation and ionized from the solid phase into the gaseous or vapor phase, and accelerated as intact molecular ions towards a detector of a mass spectrometer. The “matrix” is typically a small organic acid mixed in solution with the analyte in a 10,000:1 molar ratio of matrix/analyte. The matrix solution can be adjusted to neutral pH before mixing with the analyte.

The MALDI ionization surface may be composed of an inert material or else modified to actively capture an analyte. For example, an analyte binding partner may be bound to the surface to selectively absorb a target analyte or the surface may be coated with a thin nitrocellulose film for nonselective binding to the analyte. The surface may also be used as a reaction zone upon which the analyte is chemically modified, e.g., CNBr degradation of protein. See Bai et al, Anal. Chem. 67, 1705-1710 (1995).

Metals such as gold, copper and stainless steel are typically used to form MALDI ionization surfaces. However, other commercially-available inert materials (e.g., glass, silica, nylon and other synthetic polymers, agarose and other carbohydrate polymers, and plastics) can be used where it is desired to use the surface as a capture region or reaction zone. The use of Nation and nitrocellulose-coated MALDI probes for on-probe purification of PCR-amplified gene sequences is described by Liu et al., Rapid Commun. Mass Spec. 9:735-743 (1995). Tang et al. have reported the attachment of purified oligonucleotides to beads, the tethering of beads to a probe element, and the use of this technique to capture a complimentary DNA sequence for analysis by MALDI-TOF MS (reported by K Tang et al., at the May 1995 TOF-MS workshop, R. J. Cotter (Chairperson); K Tang et al., Nucleic Acids Res. 23, 3126-3131, 1995). Alternatively, the MALDI surface may be electrically- or magnetically activated to capture charged analytes and analytes anchored to magnetic beads respectively.

Aside from MALDI, Electrospray Ionization Mass Spectrometry (ESI/MS) has been recognized as a significant tool used in the study of proteins, protein complexes and bio-molecules in general. ESI is a method of sample introduction for mass spectrometric analysis whereby ions are formed at atmospheric pressure and then introduced into a mass spectrometer using a special interface. Large organic molecules, of molecular weight over 10,000 Daltons, may be analyzed in a quadrupole mass spectrometer using ESI.

In ESI, a sample solution containing molecules of interest and a solvent is pumped into an electrospray chamber through a fine needle. An electrical potential of several kilovolts may be applied to the needle for generating a fine spray of charged droplets. The droplets may be sprayed at atmospheric pressure into a chamber containing a heated gas to vaporize the solvent. Alternatively, the needle may extend into an evacuated chamber, and the sprayed droplets are then heated in the evacuated chamber. The fine spray of highly charged droplets releases molecular ions as the droplets vaporize at atmospheric pressure. In either case, ions are focused into a beam, which is accelerated by an electric field, and then analyzed in a mass spectrometer.

Because electrospray ionization occurs directly from solution at atmospheric pressure, the ions formed in this process tend to be strongly solvated. To carry out meaningful mass measurements, solvent molecules attached to the ions should be efficiently removed, that is, the molecules of interest should be “desolvated.” Desolvation can, for example, be achieved by interacting the droplets and solvated ions with a strong countercurrent flow (6-9 l/m) of a heated gas before the ions enter into the vacuum of the mass analyzer.

Other well-known ionization methods may also be used. For example, electron ionization (also known as electron bombardment and electron impact), atmospheric pressure chemical ionization (APCI), fast atom Bombardment (FAB), or chemical ionization (CI).

Immediately following ionization, gas phase ions enter a region of the mass spectrometer known as the mass analyzer. The mass analyzer is used to separate ions within a selected range of mass to charge ratios. This is an important part of the instrument because it plays a large role in the instrument's accuracy and mass range. Ions are typically separated by magnetic fields, electric fields, and/or measurement of the time an ion takes to travel a fixed distance.

If all ions with the same charge enter a magnetic field with identical kinetic energies a definite velocity will be associated with each mass and the radius will depend on the mass. Thus a magnetic field can be used to separate a monoenergetic ion beam into its various mass components. Magnetic fields will also cause ions to form fragment ions. If there is no kinetic energy of separation of the fragments the two fragments will continue along the direction of motion with unchanged velocity. Generally, some kinetic energy is lost during the fragmentation process creating non-integer mass peak signals which can be easily identified. Thus, the action of the magnetic field on fragmented ions can be used to give information on the individual fragmentation processes taking place in the mass spectrometer.

Electrostatic fields exert radial forces on ions attracting them towards a common center. The radius of an ion's trajectory will be proportional to the ion's kinetic energy as it travels through the electrostatic field. Thus an electric field can be used to separate ions by selecting for ions that travel within a specific range of radii which is based on the kinetic energy and is also proportion to the mass of each ion.

Quadrupole mass analyzers have been used in conjunction with electron ionization sources since the 1950s. Quadrupoles are four precisely parallel rods with a direct current (DC) voltage and a superimposed radio-frequency (RF) potential. The field on the quadrupoles determines which ions are allowed to reach the detector. The quadrupoles thus function as a mass filter. As the field is imposed, ions moving into this field region will oscillate depending on their mass-to-charge ratio and, depending on the radio frequency field, only ions of a particular m/z can pass through the filter. The m/z of an ion is therefore determined by correlating the field applied to the quadrupoles with the ion reaching the detector. A mass spectrum can be obtained by scanning the RF field. Only ions of a particular m/z are allowed to pass through.

Electron ionization coupled with quadrupole mass analyzers can be employed in practicing the instant invention. Quadrupole mass analyzers have found new utility in their capacity to interface with electrospray ionization This interface has three primary advantages. First, quadrupoles are tolerant of relatively poor vacuums (˜5×10⁻⁵ torr), which makes it well-suited to electrospray ionization since the ions are produced under atmospheric pressure conditions. Secondly, quadrupoles are now capable of routinely analyzing up to an m/z of 3000, which is useful because electrospray ionization of proteins and other biomolecules commonly produces a charge distribution below m/z 3000. Finally, the relatively low cost of quadrupole mass spectrometers makes them attractive as electrospray analyzers.

The ion trap mass analyzer was conceived of at the same time as the quadrupole mass analyzer. The physics behind both of these analyzers is very similar. In an ion trap the ions are trapped in a radio frequency quadrupole field. One method of using an ion trap for mass spectrometry is to generate ions externally with ESI or MALDI, using ion optics for sample injection into the trapping volume. The quadrupole ion trap typically consist of a ring electrode and two hyperbolic endcap electrodes. The motion of the ions trapped by the electric field resulting from the application of RF and DC voltages allows ions to be trapped or ejected from the ion trap. In the normal mode the RF is scanned to higher voltages, the trapped ions with the lowest m/z and are ejected through small holes in the endcap to a detector (a mass spectrum is obtained by resonantly exciting the ions and thereby ejecting from the trap and detecting them). As the RF is scanned further, higher m/z ratios become are ejected and detected. It is also possible to isolate one ion species by ejecting all others from the trap. The isolated ions can subsequently be fragmented by collisional activation and the fragments detected. The primary advantages of quadrupole ion traps is that multiple collision-induced dissociation experiments can be performed without having multiple analyzers. Other important advantages include its compact size, and the ability to trap and accumulate ions to increase the signal-to-noise ratio of a measurement.

Quadrupole ion traps can be used in conjunction with electrospray ionization MS/MS experiments in the instant invention.

The earliest mass analyzers separated ions with a magnetic field. In magnetic analysis, the ions are accelerated (using an electric field) and are passed into a magnetic field. A charged particle traveling at high speed passing through a magnetic field will experience a force, and travel in a circular motion with a radius depending upon the m/z and speed of the ion. A magnetic analyzer separates ions according to their radii of curvature, and therefore only ions of a given m/z will be able to reach a point detector at any given magnetic field. A primary limitation of typical magnetic analyzers is their relatively low resolution.

In order to improve resolution, single-sector magnetic instruments have been replaced with double-sector instruments by combining the magnetic mass analyzer with an electrostatic analyzer. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument to 100,000. Magnetic double-focusing instrumentation is commonly used with FAB and EI ionization, however they are not widely used for electrospray and MALDI ionization sources primarily because of the much higher cost of these instruments. But in theory, they can be employed to practice the instant invention.

ESI and MALDI-MS commonly use quadrupole and time-of-flight mass analyzers, respectively. The limited resolution offered by time-of-flight mass analyzers, combined with adduct formation observed with MALDI-MS, results in accuracy on the order of 0.1% to a high of 0.01%, while ESI typically has an accuracy on the order of 0.01%. Both ESI and MALDI are now being coupled to higher resolution mass analyzers such as the ultrahigh resolution (>10⁵) mass analyzer. The result of increasing the resolving power of ESI and MALDI mass spectrometers is an increase in accuracy for biopolymer analysis.

Fourier-transform ion cyclotron resonance (FTMS) offers two distinct advantages, high resolution and the ability to tandem mass spectrometry experiments. FTMS is based on the principle of a charged particle orbiting in the presence of a magnetic field. While the ions are orbiting, a radio frequency (RF) signal is used to excite them and as a result of this RF excitation, the ions produce a detectable image current. The time-dependent image current can then be Fourier transformed to obtain the component frequencies of the different ions which correspond to their m/z.

Coupled to ESI and MALDI, FTMS offers high accuracy with errors as low as ±0.001%. The ability to distinguish individual isotopes of a protein of mass 29,000 is demonstrated.

A time-of-flight (TOF) analyzer is one of the simplest mass analyzing devices and is commonly used with MALDI ionization. Time-of-flight analysis is based on accelerating a set of ions to a detector with the same amount of energy. Because the ions have the same energy, yet a different mass, the ions reach the detector at different times. The smaller ions reach the detector first because of their greater velocity and the larger ions take longer, thus the analyzer is called time-of-flight because the mass is determine from the ions' time of arrival.

The arrival time of an ion at the detector is dependent upon the mass, charge, and kinetic energy of the ion. Since kinetic energy (KE) is equal to ½ mv² or velocity v=(2 KE/m)^{1/2}, ions will travel a given distance, d, within a time, t, where t is dependent upon their m/z.

The magnetic double-focusing mass analyzer has two distinct parts, a magnetic sector and an electrostatic sector. The magnet serves to separate ions according to their mass-to-charge ratio since a moving charge passing through a magnetic field will experience a force, and travel in a circular motion with a radius of curvature depending upon the m/z of the ion. A magnetic analyzer separates ions according to their radii of curvature, and therefore only ions of a given m/z will be able to reach a point detector at any given magnetic field. A primary limitation of typical magnetic analyzers is their relatively low resolution. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2 V/E allows one to determine that only ions of energy V will be allowed to pass. Thus, the addition of an electric sector allows only ions of uniform kinetic energy to reach the detector, thereby increasing the resolution of the two sector instrument.

The new ionization techniques are relatively gentle and do not produce a significant amount of fragment ions, this is in contrast to electron ionization (EI) which produces many fragment ions. To generate more information on the molecular ions generated in the ESI and MALDI ionization sources, it has been necessary to apply techniques such as tandem mass spectrometry (MS/MS), to induce fragmentation. Tandem mass spectrometry (abbreviated MSn—where n refers to the number of generations of fragment ions being analyzed) allows one to induce fragmentation and mass analyze the fragment ions. This is accomplished by collisionally generating fragments from a particular ion and then mass analyzing the fragment ions.

Tandem mass spectrometry or post source decay is used for proteins that cannot be identified by peptide-mass matching or to confirm the identity of proteins that are tentatively identified by an error-tolerant peptide mass search, described above. This method combines two consecutive stages of mass analysis to detect secondary fragment ions that are formed from a particular precursor ion. The first stage serves to isolate a particular ion of a particular peptide (polypeptide) of interest based on its m/z. The second stage is used to analyze the product ions formed by spontaneous or induced fragmentation of the selected ion precursor. Interpretation of the resulting spectrum provides limited sequence information for the peptide of interest. However, it is faster to use the masses of the observed peptide fragment ions to search an appropriate protein sequence database and identify the protein as described in Griffin et al, Rapid Commun. Mass. Spectrom. 1995, 9: 1546. Peptide fragment ions are produced primarily by breakage of the amide bonds that join adjacent amino acids. The fragmentation of peptides in mass spectrometry has been well described (Falick et al., J. Am Soc. Mass Spectrom. 1993, 4, 882-893; Bieniann, K., Biomed. Environ. Mass Spectrom. 1988, 16, 99-111).

For example, fragmentation can be achieved by inducing ion/molecule collisions by a process known as collision-induced dissociation (CID) or also known as collision-activated dissociation (CAD). CID is accomplished by selecting an ion of interest with a mass filter/analyzer and introducing that ion into a collision cell. A collision gas (typically Ar, although other noble gases can also be used) is introduced into the collision cell, where the selected ion collides with the argon atoms, resulting in fragmentation. The fragments can then be analyzed to obtain a fragment ion spectrum. The abbreviation MSn is applied to processes which analyze beyond the initial fragment ions (MS2) to second (MS3) and third generation fragment ions (MS4). Tandem mass analysis is primarily used to obtain structural information, such as protein or polypeptide sequence, in the instant invention.

In certain instruments, such as those by JEOL USA, Inc. (Peabody, Mass.), the magnetic and electric sectors in any JEOL magnetic sector mass spectrometer can be scanned together in “linked scans” that provide powerful MS/MS capabilities without requiring additional mass analyzers. Linked scans can be used to obtain product-ion mass spectra, precursor-ion mass spectra, and constant neutral-loss mass spectra These can provide structural information and selectivity even in the presence of chemical interferences. Constant neutral loss spectrum essentially “lifts out” only the interested peaks away from all the background peaks, hence removing the need for class separation and purification. Neutral loss spectrum can be routinely generated by a number of commercial mass spectrometer instruments (such as the one used in the Example section). JEOL mass spectrometers can also perform fast linked scans for GC/MS/MS and LC/MS/MS experiments.

Once the ion passes through the mass analyzer it is then detected by the ion detector, the final element of the mass spectrometer. The detector allows a mass spectrometer to generate a signal (current) from incident ions, by generating secondary electrons, which are further amplified. Alternatively some detectors operate by inducing a current generated by a moving charge. Among the detectors described, the electron multiplier and scintillation counter are probably the most commonly used and convert the kinetic energy of incident ions into a cascade of secondary electrons. Ion detection can typically employ Faraday Cup, Electron Multiplier, Photomultiplier Conversion Dynode (Scintillation Counting or Daly Detector), High-Energy Dynode Detector (HED), Array Detector, or Charge (or Inductive) Detector.

The introduction of computers for MS work entirely altered the manner in which mass spectrometry was performed. Once computers were interfaced with mass spectrometers it was possible to rapidly perform and save analyses. The introduction of faster processors and larger storage capacities has helped launch a new era in mass spectrometry. Automation is now possible allowing for thousands of samples to be analyzed in a single day. Te use of computer also helps to develop mass spectra databases which can be used to store experimental results. Software packages not only helped to make the mass spectrometer more user friendly but also greatly expanded the instrument's capabilities.

The ability to analyze complex mixtures has made MALDI and ESI very useful for the examination of proteolytic digests, an application otherwise known as protein mass mapping. Through the application of sequence specific proteases, protein mass mapping allows for the identification of protein primary structure. Performing mass analysis on the resulting proteolytic fragments thus yields information on fragment masses with accuracy approaching ±5 ppm, or ±0.005 Da for a 1,000 Da peptide. The protease fragmentation pattern is then compared with the patterns predicted for all proteins within a database and matches are statistically evaluated. Since the occurrence of Arg and Lys residues in proteins is statistically high, trypsin cleavage (specific for Arg and Lys) generally produces a large number of fragments which in turn offer a reasonable probability for unambiguously identifying the target protein.

The primary tools in these protein identification experiments are mass spectrometry, proteases, and computer-facilitated data analysis. As a result of generating intact ions, the molecular weight information on the peptides/proteins are quite unambiguous. Sequence specific enzymes can then provide protein fragments that can be associated with proteins within a database by correlating observed and predicted fragment masses. The success of this strategy, however, relies on the existence of the protein sequence within the database. With the availability of the human genome sequence (which indirectly contain the sequence information of all the proteins in the human body) and genome sequences of other organisms (mouse, rat, Drosophila, C. elegans, bacteria, yeasts, etc.), identification of the proteins can be quickly determined simply by measuring the mass of proteolytic fragments.

Representative mass spectrometry instruments useful for practicing the instant invention are described in detail in the Examples. A skilled artisan should readily understand that other similar instruments with equivalent function/specification, either commercially available or user modified, are suitable for practicing the instant invention.

Protease Digestion

Prior to analysis by mass spectrometry, the protein may be chemically or enzymatically digested. For protein bands from gels, the protein sample in the gel slice may be subjected to in-gel digestion. (see Shevchenko A. et al., Mass Spectrometric Sequencing of Proteins from Silver Stained Polyacrylamide Gels. Analytical Chemistry 1996, 58: 850).

One aspect of the instant invention is that peptide fragments ending with lysine or arginine residues can be used for sequencing with tandem mass spectrometry. While trypsin is the preferred the protease, many different enzymes can be used to perform the digestion to generate peptide fragments ending with Lys or Arg residues. For instance, in page 886 of a 1979 publication of Enzymes (Dixon, M. et al. ed., 3rd edition, Academic Press, New York and San Francisco, the content of which is incorporated herein by reference), a host of enzymes are listed which all have preferential cleavage sites of either Arg- or Lys- or both, including Trypsin [EC 3.4.21.4], Thrombin [EC 3.4.21.5], Plasmin [EC 3.4.21.7], Kallikrein [EC 3.4.21.8], Acrosin [EC 3.4.21.10], and Coagulation factor Xa [EC 3.4.21.6]. Particularly, Acrosin is the Trypsin-like enzyme of spermatoza, and it is not inhibited by α1-antitrypsin. Plasmin is cited to have higher selectivity than Trypsin, while Thrombin is said to be even more selective. However, this list of enzymes are for illustration purpose only and is not intended to be limiting in any way. Other enzymes known to reliably and predictably perform digestions to generate the polypeptide fragments as described in the instant invention are also within the scope of the invention.

Sequence and Literature Databases and Database Search

The raw data of mass spectrometry will be compared to public, private or commercial databases to determine the identity of polypeptides.

BLAST search can be performed at the NCBI's (National Center for Biotechnology Information) BLAST website. According to the NCBI BLAST website, BLAST® (Basic Local Alignment Search Tool) is a set of similarity search programs designed to explore all of the available sequence databases regardless of whether the query is protein or DNA. The BLAST programs have been designed for speed, with a minimal sacrifice of sensitivity to distant sequence relationships. The scores assigned in a BLAST search have a well-defined statistical interpretation, making real matches easier to distinguish from random background hits. BLAST uses a heuristic algorithm which seeks local as opposed to global alignments and is therefore able to detect relationships among sequences which share only isolated regions of similarity (Altschul et al., 1990, J. Mol. Biol. 215: 403-10). The BLAST website also offer a “BLAST course,” which explains the basics of the BLAST algorithm, for a better understanding of BLAST.

For protein sequence search, several protein-protein BLAST can be used. Protein BLAST allows one to input protein sequences and compare these against other protein sequences.

“Standard protein-protein BLAST” takes protein sequences in FASTA format, GenBank Accession numbers or GI numbers and compares them against the NCBI protein databases (see below).

“PSI-BLAST” (Position Specific Iterated BLAST) uses an iterative search in which sequences found in one round of searching are used to build a score model for the next round of searching. Highly conserved positions receive high scores and weakly conserved positions receive scores near zero. The profile is used to perform a second (etc.) BLAST search and the results of each “iteration” used to refine the profile. This iterative searching strategy results in increased sensitivity.

“PHI-BLAST” (Pattern Hit Initiated BLAST) combines matching of regular expression pattern with a Position Specific iterative protein search PHI-BLAST can locate other protein sequences which both contain the regular expression pattern and are homologous to a query protein sequence.

“Search for short, nearly exact sequences” is an option similar to the standard protein-protein BLAST with the parameters set automatically to optimize for searching with short sequences. A short query is more likely to occur by chance in the database. Therefore increasing the Expect value threshold, and also lowering the word size is often necessary before results can be returned. Low Complexity filtering has also been removed since this filters out larger percentage of a short sequence, resulting in little or no query sequence remaining. Also for short protein sequence searches the Matrix is changed to PAM-30 which is better suited to finding short regions of high similarity.

The databases that can be searched by the BLAST program is user selected, and is subject to frequent updates at NCBI. The most commonly used ones are:

Nr: All non-redundant GenBank CDS translations+PDB+SwissProt+PIR+PRF;

Month: All new or revised GenBank CDS translation+PDB+SwissProt+PIR+PRF released in the last 30 days;

Swissprot: Last major release of the SWISS-PROT protein sequence database (no updates);

Drosophila genome: Drosophila genome proteins provided by Celera and Berkeley Drosophila Genome Project (BDGP);

S. cerevisiae: Yeast (Saccharomyces cerevisiae) genomic CDS translations;

E coli: Escherichia coli genomic CDS translations;

Pdb: Sequences derived from the 3-dimensional structure from Brookhaven Protein Data Bank;

Alu: Translations of select Alu repeats from REPBASE, suitable for masking Alu repeats from query sequences. It is available by anonymous FTP from the NCBI website. See “Alu alert” by Claverie and Makalowski, Nature vol. 371, page 752 (1994).

Some of the BLAST databases, like SwissProt, PDB and Kabat are complied outside of NCBI. Other like e coli, dbEST and month, are subsets of the NCBI databases. Other “virtual Databases” can be created using the “Limit by Entrez Query” option.

The Welcome Trust Sanger Institute offer the Ensembl software system which produces and maintains automatic annotation on eukaryotic genomes. All data and codes can be downloaded without constraints from the Sanger Centre website. The Centre also provides the Ensembl's International Protein Index databases which contain more than 90% of all known human protein sequences and additional prediction of about 10,000 proteins with supporting evidence. All these can be used for database search purposes.

In addition, many commercial databases are also available for search purposes. For example, Celera has sequenced the whole human genome and offers commercial access to its proprietary annotated sequence database (Discovery™ database).

Various softwares can be employed to search these databases. The probability search software Mascot (Matrix Science Ltd.). Mascot utilizes the Mowse search algorithm and scores the hits using a probabilistic measure (Perkins et al., 1999, Electrophoresis 20: 3551-3567, the entire contents are incorporated herein by reference). The Mascot score is a function of the database utilized, and the score can be used to assess the null hypothesis that a particular match occurred by chance. Specifically, a Mascot score of 46 implies that the chance of a random hit is less than 5%. However, the total score consists of the individual peptide scores, and occasionally, a high total score can derive from many poor hits. To exclude this possibility, only “high quality” hits—those with a total score >46 with at least a single peptide match with a score of 30 ranking number 1—are considered.

Other similar softwares can also be used according to manufacturer's suggestion.

PubMed, available via the NCBI Entrez retrieval system, was developed by the National Center for Biotechnology Information (NCBI) at the National Library of Medicine (NLM), located at the National Institutes of Health (NIH). The PubMed database was developed in conjunction with publishers of biomedical literature as a search tool for accessing literature citations and linking to full-text journal articles at web sites of participating publishers.

Publishers participating in PubMed electronically supply NLM with their citations prior to or at the time of publication. If the publisher has a web site that offers full-text of its journals, PubMed provides links to that site, as well as sites to other biological data, sequence centers, etc. User registration, a subscription fee, or some other type of fee may be required to access the full-text of articles in some journals.

In addition, PubMed provides a Batch Citation Matcher, which allows publishers (or other outside users) to match their citations to PubMed entries, using bibliographic information such as journal, volume, issue, page number, and year. This permits publishers easily to link from references in their published articles directly to entries in PubMed.

PubMed provides access to bibliographic information which includes MEDLINE as well as:

The out-of-scope citations (e.g., articles on plate tectonics or astrophysics) from certain MEDLINE journals, primarily general science and chemistry journals, for which the life sciences articles are indexed for MEDLINE.

Citations that precede the date that a journal was selected for MEDLINE indexing.

Some additional life science journals that submit full text to PubMed Central and receive a qualitative review by NLM.

PubMed also provides access and links to the integrated molecular biology databases included in NCBI's Entrez retrieval system. These databases contain DNA and protein sequences, 3-D protein structure data, population study data sets, and assemblies of complete genomes in an integrated system.

MEDLINE is the NLM's premier bibliographic database covering the fields of medicine, nursing, dentistry, veterinary medicine, the health care system, and the pre-clinical sciences. MEDLINE contains bibliographic citations and author abstracts from more than 4,300 biomedical journals published in the United States and 70 other countries. The file contains over 11 million citations dating back to the mid-1960's. Coverage is worldwide, but most records are from English-language sources or have English abstracts.

PubMed's in-process records provide basic citation information and abstracts before the citations are indexed with NLM's MeSH Terms and added to MEDLINE. New in process records are added to PubMed daily and display with the tag [PubMed—in process]. After MeSH terms, publication types, GenBank accession numbers, and other indexing data are added, the completed MEDLINE citations are added weekly to PubMed.

Citations received electronically from publishers appear in PubMed with the tag [PubMed—as supplied by publisher]. These citations are added to PubMed Tuesday through Saturday. Most of these progress to In Process, and later to MEDLINE status. Not all citations will be indexed for MEDLINE and are tagged, [PubMed—as supplied by publisher].

The Batch Citation Matcher allows users to match their own list of citations to PubMed entries, using bibliographic information such as journal, volume, issue, page number, and year. The Citation Matcher reports the corresponding PMID. This number can then be used to easily to link to PubMed. This service is frequently used by publishers or other database providers who wish to link from bibliographic references on their web sites directly to entries in PubMed.

Separation of Polypeptide Complexes

Polypeptide separation schemes can achieved based on differences in the molecular properties such as size, charge and solubility. Protocols based on these parameters include SDS-PAGE (SDS-PolyAcrylamide Gel Electrophoresis), size exclusion chromatography, ion exchange chromatography, differential precipitation and the like. SDS-PAGE is well-known in the art of biology, and will not be described here in detail. See Molecular Cloning A Laboratory Manual, 2nd Ed., ed. by Sambrook, Fritsch and Maniatis (Cold Spring Harbor Laboratory Press: 1989).

Size exclusion chromatography, otherwise known as gel filtration or gel permeation chromatography, relies on the penetration of macromolecules in a mobile phase into the pores of stationary phase particles. Differential penetration is a function of the hydrodynamic volume of the particles. Accordingly, under ideal conditions the larger molecules are excluded from the interior of the particles while the smaller molecules are accessible to this volume and the order of elution can be predicted by the size of the polypeptide because a linear relationship exists between elution volume and the log of the molecular weight. Size exclusion chromatographic supports based on cross-linked dextrans e.g. SEPHADEX.RTM., spherical agarose beads e.g. SEPHAROSE.RTM. (both commercially available from Pharmacia AB. Uppsala, Sweden), based on cross-linked polyacrylamides e.g. BIO-GEL.RTM. (commercially available from BioRad Laboratories, Richmond, Calif.) or based on ethylene glycol-methacrylate copolymer e.g. TOYOPEARL HW65S (commercially available from ToyoSoda Co., Tokyo, Japan) are useful in the practice of this invention.

Precipitation methods are predicated on the fact that in crude mixtures of polypeptides the solubilities of individual polypeptides are likely to vary widely. Although the solubility of a polypeptide in an aqueous medium depends on a variety of factors, for purposes of this discussion it can be said generally that a polypeptide will be soluble if its interaction with the solvent is stronger than its interaction with polypeptide molecules of the same or similar kind. Without wishing to be bound by any particular mechanistic theory describing precipitation phenomena, it is nonetheless believed that the interaction between a polypeptide and water molecules occur by hydrogen bonding with several types of charged groups, and electrostatically as dipoles with uncharged groups, and that precipitants such as salts of monovalent cations (e.g., ammonium sulfate) compete with polypeptides for water molecules, thus at high salt concentrations, the polypeptides become “dehydrated” reducing their interaction with the aqueous environment and increasing the aggregation with like or similar polypeptides resulting in precipitation from the medium.

Ion exchange chromatography involves the interaction of charged functional groups in the sample with ionic functional groups of opposite charge on an adsorbent surface. Two general types of interaction are known. Anionic exchange chromatography mediated by negatively charged amino acid side chains (e.g. aspartic acid and glutamic acid) interacting with positively charged surfaces and cationic exchange chromatography mediated by positively charged amino acid residues (e.g. lysine and arginine) interacting with negatively charged surfaces.

More recently affinity chromatography and hydrophobic interaction chromatography techniques have been developed to supplement the more traditional size exclusion and ion exchange chromatographic protocols. Affinity chromatography relies on the interaction of the polypeptide with an immobilized ligand. The ligand can be specific for the particular polypeptide of interest in which case the ligand is a substrate, substrate analog, inhibitor or antibody. Alternatively, the ligand may be able to react with a number of polypeptides. Such general ligands as adenosine monophosphate, adenosine diphosphate, nicotine adenine dinucleotide or certain dyes may be employed to recover a particular class of polypeptides. One of the least biospecific of the affinity chromatographic approaches is immobilized metal affinity chromatography (IMAC), also referred to as metal chelate chromatography. IMAC introduced by Porath et al.(Nature 258:598-99(1975) involves chelating a metal to a solid support and then forming a complex with electron donor amino acid residues on the surface of a polypeptide to be separated.

Hydrophobic interaction chromatography was first developed following the observation that polypeptides could be retained on affinity gels which comprised hydrocarbon spacer arms but lacked the affinity ligand. Although in this field the term hydrophobic chromatography is sometimes used, the term hydrophobic interaction chromatography (HIC) is preferred because it is the interaction between the solute and the gel that is hydrophobic not the chromatographic procedure. Hydrophobic interactions are strongest at high ionic strength, therefore, this form of separation is conveniently performed following salt precipitations or ion exchange procedures. Elution from HIC supports can be effected by alterations in solvent, pH, ionic strength, or by the addition of chaotropic agents or organic modifiers, such as ethylene glycol. A description of the general principles of hydrophobic interaction chromatography can be found in U.S. Pat. No. 3,917,527 and in U.S. Pat. No. 4,000,098. The application of HIC to the purification of specific polypeptides is exemplified by reference to the following disclosures: human growth hormone (U.S. Pat. No. 4,332,717), toxin conjugates (U.S. Pat. No. 4,771,128), antihemolytic factor (U.S. Pat No. 4,743,680), tumor necrosis factor (U.S. Pat. No. 4,894,439), interleukin-2 (U.S. Pat. No. 4,908,434), human lymphotoxin (U.S. Pat. No. 4,920,196) and lysozyme species (Fausnaugh, J. L. and F. E. Regnier, J. Chromatog. 359:131-146 (1986)).

The principles of IMAC are generally appreciated. It is believed that adsorption is predicated on the formation of a metal coordination complex between a metal ion, immobilized by chelation on the adsorbent matrix, and accessible electron donor amino acids on the surface of the polypeptide to be bound. The metal-ion microenvironment including, but not limited to, the matrix, the spacer arm, if any, the chelating ligand, the metal ion, the properties of the surrounding liquid medium and the dissolved solute species can be manipulated by the skilled artisan to affect the desired fractionation.

Not wishing to be bound by any particular theory as to mechanism, it is further believed that the more important amino acid residues in terms of binding are histidine, tryptophan and probably cysteine. Since one or more of these residues are generally found in polypeptides, one might expect all polypeptides to bind to IMAC columns. However, the residues not only need to be present but also accessible (e.g., oriented on the surface of the polypeptide) for effective binding to occur. Other residues, for example poly-histidine tails added to the amino terminus or carboxyl terminus of polypeptides, can be engineered into the recombinant expression systems by following the protocols described in U.S. Pat No. 4,569,794.

The nature of the metal and the way it is coordinated on the column can also influence the strength and selectivity of the binding reaction. Matrices of silica gel, agarose and synthetic organic molecules such as polyvinyl-methacrylate co-polymers can be employed. The matrices preferably contain substituents to promote chelation. Substituents such as iminodiacetic acid (IDA) or its tris (carboxymethyl) ethylene diamine (TED) can be used. IDA is preferred. A particularly useful IMAC material is a polyvinyl methacrylate co-polymer substituted Keith IDA available commercially, e.g., as TOYOPEARL AF-CHELATE 650M (ToyoSoda Co.; Tokyo. The metals are preferably divalent members of the first transition series through to zinc, although Co⁺⁺, Ni⁺⁺, Cd⁺⁺ and Fe⁺⁺⁺ can be used. An important selection parameter is, of course, the affinity of the polypeptide to be purified for the metal. Of the four coordination positions around these metal ions, at least one is occupied by a water molecule which is readily replaced by a stronger electron donor such as a histidine residue at slightly alkaline pH.

In practice the IMAC column is “charged” with metal by pulsing with a concentrated metal salt solution followed by water or buffer. The column often acquires the color of the metal ion (except for zinc). Often the amount of metal is chosen so that approximately half of the column is charged. This allows for slow leakage of the metal ion into the non-charged area without appearing in the eluate. A pre-wash with intended elution buffers is usually carried out. Sample buffers may contain salt up to 1M or greater to minimize nonspecific ion-exchange effects. Adsorption of polypeptides is maximal at higher pHs. Elution is normally either by lowering of pH to protonate the donor groups on the adsorbed polypeptide, or by the use of stronger complexing agent such as imidazole, or glycine buffers at pH 9. In these latter cases the metal may also be displaced from the column. Linear gradient elution procedures can also be beneficially employed.

As mentioned above, IMAC is particularly useful when used in combination with other polypeptide fractionation techniques. That is to say it is preferred to apply IMAC to material that has been partially fractionated by other protein fractionation procedures. A particularly useful combination chromatographic protocol is disclosed in U.S. Pat. No. 5,252,216 granted Oct. 12, 1993, the contents of which are incorporated herein by reference. It has been found to be useful, for example, to subject a sample of conditioned cell culture medium to partial purification prior to the application of IMAC. By the term “conditioned cell culture medium” is meant a cell culture medium which has supported cell growth and/or cell maintenance and contains secreted product. A concentrated sample of such medium is subjected to one or more polypeptide purification steps prior to the application of a IMAC step. The sample may be subjected to ion exchange chromatography as a first step. As mentioned above various anionic or cationic substituents may be attached to matrices in order to form anionic or cationic supports for chromatography. Anionic exchange substituents include diethylaminoethyl (DEAE), quaternary aminoethyl (QAE) and quaternary amine (Q) groups. Cationic exchange substituents include carboxymethyl (CM), sulfoethyl (SE), sulfopropyl (SP), phosphate (P) and sulfonate (S). Cellulosic ion exchange resins such as DE23, DE32, DE52, CM-23, CM-32 and CM-52 are available from Whatman Ltd. Maidstone, Kent, U.K SEPHADEX.RTM.-based and cross-linked ion exchangers are also known. For example, DEAE-, QAE-, CM-, and SP-dextran supports under the tradename SEPHADEX.RTM. and DEAE-, Q-, CM-and S-agarose supports under the tradename SEPHAROSE.RTM. are all available from Pharmacia AB. Further both DEAE and CM derivitized ethylene glycol-methacrylate copolymer such as TOYOPEARL DEAE-650S and TOYOPEARL CM-650S are available from Toso Haas Co., Philadelphia, Pa. Because elution from ionic supports sometimes involves addition of salt and IMAC may be enhanced under increased salt concentrations. The introduction of a IMAC step following an ionic exchange chromatographic step or other salt mediated purification step may be employed. Additional purification protocols may be added including but not necessarily limited to HIC, further ionic exchange chromatography, size exclusion chromatography, viral inactivation, concentration and freeze drying.

Hydrophobic molecules in an aqueous solvent will self-associate. This association is due to hydrophobic interactions. It is now appreciated that macromolecules such as polypeptides have on their surface extensive hydrophobic patches in addition to the expected hydrophilic groups. HIC is predicated, in part, on the interaction of these patches with hydrophobic ligands attached to chromatographic supports. A hydrophobic ligand coupled to a matrix is variously referred to herein as an HIC support, HIC gel or HIC column. It is further appreciated that the strength of the interaction between the polypeptide and the HIC support is not only a function of the proportion of non-polar to polar surfaces on the polypeptide but by the distribution of the non-polar surfaces as well.

A number of matrices may be employed in the preparation of HIC columns, the most extensively used is agarose. Silica and organic polymer resins may be used. Useful hydrophobic ligands include but are not limited to alkyl groups having from about 2 to about 10 carbon atoms, such as a butyl, propyl, or octyl; or aryl groups such as phenyl. Conventional HIC products for gels and columns may be obtained commercially from suppliers such as Pharmacia LKB AB, Uppsala, Sweden under the product names butyl-SEPHAROSE.RTM., phenyl-SEPHAROSE.RTM. CL-4B, octyl-SEPHAROSE.RTM. FF and phenyl-SEPHAROSE.RTM. FF; Tosoh Corporation, Tokyo, Japan under the product names TOYOPEARL Butyl 650, Ether-650, or Phenyl-650 (FRACTOGEL TSK Butyl-650) or TSK-GEL phenyl-5PW; Miles-Yeda, Rehovot, Israel under the product name ALKYL-AGAROSE, wherein the alkyl group contains from 2-10 carbon atoms, and J. T. Baker, Phillipsburg, N.J. under the product name BAKERBOND WP-HI-propyl.

Ligand density is an important parameter in that it influences not only the strength of the interaction but the capacity of the column as well. The ligand density of the commercially available phenyl or octyl phenyl gels is on the order of 40 μM/ml gel bed. Gel capacity is a function of the particular polypeptide in question as well pH, temperature and salt concentration but generally can be expected to fall in the range of 3-20 mg/ml of gel.

The choice of a particular gel can be determined by the skilled artisan. In general the strength of the interaction of the polypeptide and the HIC ligand increases with the chain length of the of the alkyl ligands but ligands having from about 4 to about 8 carbon atoms are suitable for most separations. A phenyl group has about the same hydrophobicity as a pentyl group, although the selectivity can be quite different owing to the possibility of pi-pi interaction with aromatic groups on the polypeptide.

Adsorption of the polypeptides to a HIC column is favored by high salt concentrations, but the actual concentrations can vary over a wide range depending on the nature of the polypeptide and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba⁺⁺<Ca⁺⁺<Mg⁺⁺<Li⁺<Cs⁺<Na⁺<K⁺<Rb⁺<NH₄⁺. While anions may be ranked in terms of increasing chaotropic effect as PO₄⁻⁻⁻<SO₄⁻⁻<CH₃COO⁻<Cl⁻<Br⁻<NO₃⁻<CIO₄⁻<I⁻<SCN⁻.

Accordingly, salts may be formulated that influence the strength of the interaction as given by the following relationship:

Na₂SO₄>NaCl>(NH₄)₂SO₄>NH₄Cl>NaBr>NaSCN

In general, salt concentrations of between about 0.75 and about 2M ammonium sulfate or between about 1 and 4M NaCl are useful.

The influence of temperature on HIC separations is not simple, although generally a decrease in temperature decreases the interaction However, any benefit that would accrue by increasing the temperature must also be weighed against adverse effects such an increase may have on the activity of the polypeptide.

Elution, whether stepwise or in the form of a gradient, can be accomplished in a variety of ways: (a) by changing the salt concentration, (b) by changing the polarity of the solvent or (c) by adding detergents. By decreasing salt concentration adsorbed polypeptides are eluted in order of increasing hydrophobicity. Changes in polarity may be affected by additions of solvents such as ethylene glycol or (iso)propanol thereby decreasing the strength of the hydrophobic interactions. Detergents function as displacers of polypeptides and have been used primarily in connection with the purification of membrane polypeptides.

When the eluate resulting from HIC is subjected to further ion exchange chromatography, both anionic and cationic procedures may be employed.

As mentioned above, gel filtration chromatography affects separation based on the size of molecules. It is in effect a form of molecular sieving. It is desirable that no interaction between the matrix and solute occur, therefore, totally inert matrix materials are preferred. It is also desirable that the matrix be rigid and highly porous. For large scale processes rigidity is most important as that parameter establishes the overall flow rate. Traditional materials such as crosslinked dextran or polyacrylamide matrices, commercially available as, e.g., SEPHADEX.RTM. and BIOGEL.RTM., respectively, were sufficiently inert and available in a range of pore sizes, however these gels were relatively soft and not particularly well suited for large scale purification. More recently, gels of increased rigidity have been developed (e.g. SEPHACRYL.RTM., ULTROGEL.RTM., FRACTOGEL.RTM. and SUPEROSE.RTM.). All of these materials are available in particle sizes which are smaller than those available in traditional supports so that resolution is retained even at higher flow rates. Ethylene glycol-methacrylate copolymer matrices, e.g., such as the TOYOPEARL HW series matrices (Toso Haas) are preferred.

Phosphoproteins can be isolated using IMAC as described above. However, they can also be isolated by other means. Specifically, phosphoproteins with phosphorylated tyrosine residues can be isolated with phospho-tyrosine specific antibodies. Likewise, phospho-serine/threonine specific antibodies can be used to isolate phosphoproteins with phosphorylated serine/threonine residues. Many of these antibodies are available as affinity purified forms, either as monoclonal antibodies or antisera or mouse ascites fluid. For example, phospho-Tyrosine monoclonal antibody (P-Tyr-102) is a high-affinity IgG1 phospho-tyrosine antibody clone that is produced and characterized by Cell Signaling Technology (Beverly, Mass.). As determined by ELISA, P-Tyr-102 (Cat. No. 9416) binds to a larger number of phospho-tyrosine containing peptides in a manner largely independent of the surrounding amino acid sequences, and also interacts with a broader range of phospho-tyrosine containing polypeptides as indicated by 2D-gel Western analysis. P-Tyr-102 is highly specific for phospho-Tyr in peptides/proteins, shows no cross-reactivity with the corresponding nonphosphorylated peptides and does not react with peptides containing phospho-Ser or phospho-Thr instead of phospho-Tyr. It is expected that P-Tyr-102 will react with peptides/proteins containing phospho-Tyr from all species.

Phospho-threonine antibodies are also available. For example, Cell Signaling Technology also offer an affinity-purified rabbit polyclonal phospho-threonine antibody (P-Thr-Polyclonal, Cat. No. 9381) which binds threonine-phosphorylated sites in a manner largely independent of the surrounding amino acid sequence. It recognizes a wide range of threonine-phosphorylated peptides in ELISA and a large number of threonine-phosphorylated polypeptides in 2D analysis. It is specific for peptides/proteins containing phospho-Thr and shows no cross-reactivity with corresponding nonphosphorylated sequences. Phospho-Threonine Antibody (P-Thr-Polyclonal) does not cross-react with sequences containing either phospho-Tyrosine or phospho-Serine. It is expected that this antibody will react with threonine-phosphorylated peptides/proteins regardless of species of origin. Upstate Biotechnology (Lake Placid, N.Y.) also provides an anti-phospho-serine/threonine antibody with broad immunoreactivity for polypeptides containing phosphorylated serine and phosphorylated threonine residues.

Many other similar products are also available on the market. These antibodies can be readily coupled to supporting matrix materials to generate affinity columns according to standard molecular biology protocols (for details and general means of antibody production, see Using Antibodies: A Laboratory Manual: Portable Protocol NO. I, Harlow and Lane, Cold Spring Harbor Laboratory Press: 1998; also see Antibodies : A Laboratory Manual, edited by Harlow and Lane, Cold Spring Harbor Laboratory Press: 1988).

A similar approach can be applied towards the isolation of any specific polypeptide, against which specific antibodies are available.

Isolation of membrane-associated polypeptides can be carried out using appropriate methods as described above (for example, hydrophobic interaction chromatography). Alternatively, it can be performed with other standard molecular biology protocols. See, for example, Molecular Cloning A Laboratory Manual, 2nd Ed., ed. by Sambrook, Fritsch and Maniatis (Cold Spring Harbor Laboratory Press: 1989); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987).

For example, cells can be lysed in appropriate buffers and the membrane portions can be isolated by centrifugation. Depending on particular cases, cells preferably can be lysed in hypotonic buffer by homogenization. Cell debris and nuclei can then be removed by low speed centrifugation, followed by high speed centrifugation (such as under centrifugation conditions of 100,000×g or more) to pellet membrane portions. Membrane polypeptides can then be extracted by organic solvents such as chloroform and methanol.

Alternatively, membrane polypeptides can be isolated by extraction of membrane portions with extraction buffer containing detergents. Depending on specific occasions, the detergent used can be SDS or other ionic or non-ionic detergents. Different choices of detergent or extraction buffer in general may facilitate global non-biased extraction of membrane polypeptides or isolation of specific membrane polypeptides of interest. The reduced complexity of polypeptide mixtures resulting from the use of specific extraction protocols may be beneficial for the following digestion, separation, and analysis procedures.

A most preferred method of isolating hydrophobic membrane proteins is strong cation exchange (SCX) chromatography. Strong cation exchange (SCX) chromatography is particularly suited for isolating/purifying hydrophobic proteins, such as membrane proteins. Many SCX chromatographic columns are commercially available. For illustration purpose only, details regarding one type of SCX column, the PolySulfoethyl Aspartamide Strong Cation Exchange Columns manufactured by The Nest Group, Inc. (45 Valley Road, Southborough, Mass.), are described below. It is to be understood that the recommendations below are by no means limiting in any respect. Many other commercial SCX columns are also available, and should be used according to the recommendation of respective manufacturers.

According to the manufacturer, aspartamide cation exchange chemistries are some of the best materials available for the HPLC separation of peptides. These are wide-pore (300 Å) silica packings with a bonded coating of hydrophilic, sulfoethyl anionic polymer. With the PolySULFOETHYL Aspartamide SCX column, mobile phase modifiers can be used to help improve peptide solubility or to mediate the interaction between peptide and stationary phase. By varying the pH, ionic strength or organic solvent concentration in the mobile phase, chromatographic selectivity can be significantly enhanced. For more strongly hydrophobic peptides, a non-ionic surfactant (at a concentration below its CMC) and/or acetonitrile or n-propanol as mobile phase modifiers, can substantially improve resolution and recovery over conventional reverse phase methods. Additional selectivity can be obtained by simply changing the slope of the KCl or (NH₄)₂SO₄ gradient.

Using this column at pH 3 is better for retention of neutral to slightly acidic peptides. Use of a higher pH may be considered for basic hydrophobic peptides. The addition of MeCN or propanol to the A&B solvents (see below) changes the mechanism of separation and results in a separation based not only on positive charge, but also on hydrophobicity.

These columns are quite useful for neuropeptides, growth factors, CNBr peptide fragments, and synthetic peptides as a complement to RPC (Reverse Phase Chromatography), or to remove organic reagents from peptide samples which would cause smearing on a RPC column.

The operating conditions for these applications for an analytical column are:

Buffer A: 5 mM K-PO₄+25% MeCN;

Buffer B: 5 mM K-PO₄+25% MeCN+300-500 mM KCl;

Linear gradient, 30 min at 1 ml/min.

The peptides are retained on the column by the positive charge of at least the terminus amino and elute by total charge, charge distribution and hydrophobicity. If the peptide does not stick to the column, prepare the peptide in a small amount of buffer, or decrease the concentration of organic in the A&B solvents to 5 or 10%. Organic solvent concentration is empirically determined and n-propanol can be substituted for MeCN for more hydrophobic species.

Since the total binding capacity of these columns is on the order of 100 mg/gm of packing (for nonresolved materials) there will be a considerable Donan effect present. It will be necessary to have the sample in 5-15 mM of salt or buffer to prevent exclusion from the column. Additionally, the gradient at the outlet of the column will be much more concave than that observed on the chart paper. It is recommended that an upper load limit of 1 milligram for an analytical column. For a guard column used as a methods development column, a load limit of one-tenth of a milligram is recommended.

Flow rates of 0.7 to 1.0 ml/min with a 30 minutes gradient should be used for the analytical column. If using the 4.6×20 mm guard column as a methods development column, gradient times should be shortened to 8-10 min at the same flow rate since the void volume is only 0.3 ml. The semiprep columns, 9.4 mm ID, require flow rates and equilibration volumes 4× that of the analytical columns.

Typically, for the first run, equilibrate the analytical column in the high salt (or final pH) solution (at least 25 ml, or for a guard column used as a methods development column use 8 ml, or on the semiprep column use 100 ml), and inject the sample under these isocratic conditions to observe the elution profile. The protein should elute at the void volume. Then equilibrate the column in low salt (or low pH if doing a pH gradient) conditions and run the gradient to the final conditions. Comparison of the chromatograms will assure that the proteins will elute in a predictable fashion. To decrease elution times increase the salt concentration (in a convex or step manner), increase the pH, or shorten the equilibration times between gradient runs. Exposure to a pH above 7 should be avoided since this will affect the silica support and will shorten column life, as will temperatures above 45° C. For buffer gradients, phosphate or bis-tris are good buffers to use since they allow monitoring in the low UV range. For salt gradients, acetate salts are frequently used. However, it may be necessary to use sulfate or chloride if the buffering capacity of acetate is undesirable or if the absorbance is to be monitored below 235 nm. When chloride has been used for salt gradient elution, flush the column with at least 30 ml of deionized water at the end of the day to prevent corrosion. If a denaturant such as 4M urea is used in the mobile phase to increase the accessibility of the ionizable groups, be sure to have a silica saturator column in line in front of the injector, to minimize attack of the silica on the ion exchange column.

New columns should be condition before use, preferably according to the following protocol. Specifically, columns are filled with methanol when shipped so the (analytical) column should be flushed with at least 40 ml water before elution with salt solution to prevent precipitation The hydrophilic coating imbibes a layer of water. The resultant swelling of the coating leads to a slight and irreversible increase in the column back pressure. Some additional swelling occurs with extended use of the column. Since the swelling increases the surface area of the coating, the capacity of the column for proteins increases as well. Thus, retention times may increase by up to 10%. This process should be hastened by eluting the column with a strong buffer for at least one hour prior to its initial use. A convenient solution to use is 0.2 M monosodium phosphate +0.3 M sodium acetate.

The conditioning process is reversed by exposing the column to pure organic solvents. Accordingly, to minimize the time to start the column after a 1-2 day storage, the column should be flushed with at least 40 ml of deionized water (not methanol), and the ends should be plugged. For extended storage it is recommended that a 100% methanol storage be used to prevent bacterial growth and contamination. Exercise care when using organic solvents to prevent precipitation of salts.

It is recommended that a new column be conditioned with two injections of an inexpensive protein (e.g. BSA) before it is used to analyze very dilute or expensive samples since new HPLC columns sometimes absorb small quantities of proteins in a nonspecific manner. The sintered metal frits have been implicated in this process. Fortunately these sites are quickly saturated. Mobile phases should be filtered before use, as should samples. Failure to do so may cause the inlet frit to plug. A guard column, P410-2SEA, will prevent damage to the analytical or preparative columns. Use of 0.1% TFA or high concentrations of formic acid in the mobile phase is not recommended.

For use in normal phase and HILIC polarity, the following should be taken into consideration. By adding even more organic solvent to the mobile phase, these columns offer enough flexibility so that they may be used in a normal or Hydrophilic Interaction (HILIC) mode. Here, more polar peptides having little or no retention under conventional reverse-phase or even ion-exchange conditions are retained, and very hydrophobic peptides may have enhanced solubility and thus chromatograph better. There are two approaches to this mode: 1) using isocratic HILIC conditions or 2) using a sodium perchlorate gradient. The key to achieving HILIC conditions is to use greater than 70% organic solvent with the SCX column. Care should be taken to assure solubility of salts under these conditions.

Automation and High Throughput Screening

The methods of the present invention may be conducted in a high throughput fashion and/or by automation. One non-limiting example of high throughput is repeating a method, or variations of a method, a substantial number of times more quickly than would be possible using standard laboratory techniques. In many instances, the method is used with different samples. By a high throughput method, a single or several individuals may process about 5, 10, 25, 50, 75, 100, 250, 500, 750, 1000, 5000, or 10,000 times the number of samples than the same number of individuals would be able to process in the same time period (one, three, seven, 30, 60, 90 days).

Automation has been used to achieve high throughput. In regard to automation of the present subject methods, a variety of instrumentation may be used. In general, automation, as used in reference to the subject method, involves having instrumentation complete one or more of the operative steps that must be repeated a multitude of times in performing the method with different samples. Examples of automation include, without limitation, having instrumentation complete coupling of anti-tag antibodies to a solid support, adding the extract to an assay environment or other vessel, washings, loading of samples for separation followed by mass spectrometry of eluted polypeptides, and data collection/analysis, etc.

There is a range of automation possible for the present invention For example, the subject methods may be wholly automated or only partially automated. If wholly automated, the method may be completed by the instrumentation without any human intervention after initiating it, other than refilling reagent bottles or monitoring or programming the instrumentation as necessary. In contrast, partial automation of the subject method involves some robotic assistance with the physical steps of the method, such as mixing, washing and the like, but still requires some human intervention other than just refilling reagent bottles or monitoring or programming the instrumentation.

For example, in a preferred embodiment, the methods of the instant invention may be performed in a modular fashion. Specifically, it may include: (a) a module for retrieving recombinant clones encoding bait proteins; (b) an automated immunoprecipitation module for purification of complexes comprising bait and prey proteins; (c) an analysis module for further purifying the proteins from (b) or preparing fragments of such proteins that are suitable for mass spectrometry; (d) a mass spectrometer module for automated analysis of fragments from (c); (d) a computer module comprising an integration software for communication among the modules of the system and integrating operations; and (e) a module for performing an automated method of the invention.

Several computer implemented methods for managing HTS-process information are known. Most automated lab systems have software that takes care of scheduling samples through the system. The technician sets up the scientific method to be executed. These methods denote the exact steps that are to be performed on a single sample. A technician then executes a scheduling algorithm on a particular number of samples which determines the sample step interleaving. These scheduler must balance the load, prevent deadlocks and enforce resource use and availability.

Automated lab systems today are known as Laboratory Information Management Systems (LIMS). LIMS typically involve the integration of automated robots into a central computing system allowing for control of the processes of each work-unit involved. An example of such a LIMS is described in U.S. Pat. No. 5,985,214 (incorporated herein by reference) wherein a system and a method for rapidly identifying chemicals in liquid samples is described. The system focuses on the rapid processing of addressable sample wells or the routing of these addressable wells.

LIMS typically include sample automation and data automation. Sample automation primarily involves control of robotics processes, routing of samples and sample tracking. Data automation typically involves generation of data accumulated from a wide variety of sources. WO 99/05591 (incorporated herein by reference) describes a system and method for organizing information relating to polymer probe array chips whereby a database model is provided which organizes information relating to sample preparation, chip layout, application of samples to chips, scanning of chips, expression analysis of chip results, etc. This system models the specific high throughput entities as if the testing would be performed manually. WO 02/065334 A1 (incorporated herein by reference) provides a computer-implemented method for managing information relating to a high throughput screening (HTS) process and to apparatuses or robot means controlled by said method. A database model is provided which organizes information relating to analytes, biological targets, HTS supports, HTS conditions, interaction results, robotics steering and control, etc. WO 02/49761 A2 (incorporated herein by reference) also provides an automated laboratory system and method allow high-throughput and fully automated processing of materials, such as liquids including genetic materials. It includes a variety of aspects that may be combined into a single system. For example, processing may be performed by a plurality of robotic-equipped modular stations, where each modular station has its own unique environment in which processes are performed. Transport devices, such as conveyor belts, may move objects between modular stations, saving movement for robots in the modular stations. Gels used for gel electrophoresis may be extruded, thus decreasing the time needed to form such gels. Robotically-operated well forming tools allow wells to be formed in gels in a registered and accurate way.

WO 02/068157 A2 provides grasping mechanisms, gripper apparatus/systems, and related methods, which is useful for accurate positioning of an object (such as a microtiter plate) for automated processing. Grasping mechanisms that include stops, support surfaces, and height adjusting surfaces to determine three translational axis positions of a grasped object are provided. In addition, grasping mechanisms that are resiliently coupled to other gripper apparatus components are also provided.

Steps related to the invention, as well as alternative means of accomplishing the same or similar goals are illustrated herein. Although yeast was used in the example that follows, it should also be noted that such technique is not limited to yeast. With minor modification, very similar procedures as described below can be used for similar assays in higher eukaryotes, including mammalian cells, such as human cells.

The following non-limiting example is illustrative of the present invention.

### EXAMPLE

The following materials and methods were used in the studies described in the Example:

Materials and Methods

The base vector used for the example shown below, MT2250, was constructed as follows. FLAG-tagged yeast open reading frames (ORFs) were cloned using the Gateway™ recombination-based cloning system (Invitrogen). A galactose-inducible, C-terminal FLAG tag Gateway™ destination vector, called pGAL1-CFLAG, was constructed by inserting annealed FLAG-1/2 oligonucleotides (FLAG-1: 5′-GATCCCCCGGGATGGATTACAAGGATGACGA-CGATAAGTAACTGCA-3′ (SEQ ID NO: 1), FLAG-2: 5′-GTTATCCGCCCGG-GCTCTTATCGTCGTCATCCTTGTAATCCATCCCGGGG-3′ (SEQ ID NO: 2); FLAG=DYKDDDDL (SEQ ID NO: 3), Sigma-Aldrich) into a <GAL1 LEU2 CEN> base vector (MT2250) cut with BamHI and PstI, followed by insertion of conversion cassette B into the SmaI site. A doxycyclin-inducible C-terminal FLAG tag Gateway™ destination vector, called ptet-CFLAG, was constructed by inserting the conversion cassette B-FLAG tag region from pGAL1-CFLAG, removed as a SpeI-ClaI fragment, into pCM251 between the BamHI site and ClaI site². Both donor vectors were propagated in the E. coli DB3.1 strain to prevent lethality of the ccdB gene in the Gateway™ conversion cassette. Yeast ORFs were amplified by PCR using a 5′ primer that included the attB1 recombinational site (5′-GGGGACAAGTTTGTACAAAAAAGCAGGCTTA-3′, SEQ ID NO: 4), followed by the start codon and 18-24 bp of gene-specific sequence and a 3′ primer that included the attB2 recombinational site (5′-GGGGACCACTTTGTACAAGAAAGCTGGGTC-3′, SEQ ID NO: 5) followed by 18-24 bp of gene-specific sequence immediately upstream of the stop codon. PCR amplification was performed with Platinum Taq Hi Fidelity DNA polymerase protocol using 100 ng of S288C yeast genomic DNA. PCR products were purified using a Millipore Multiscreen-PCR system and inserted into pGAL1-CFLAG using recombinational cloning as recommended (Invitrogen).

Proteins cloned using vectors such as this, and subsequently expressed in suitable hosts, are used as bait proteins.

Yeast Culture

The yeast strains used in this study were YP1 and YP2. YP1 was strain BY4472 pep4ΔkanR from the deletion consortium (Winzeler, 1999). Strain YP2 was strain YP1 deleted for TRP1 using the plasmid, pTH4 which replaces the TRP1 gene with the HIS3 gene so that the resulting strain in trp⁻, HIS⁺ (Cross, 1997). General yeast biology techniques are common knowledge and will not be recited. XY medium contains 2% bactopeptone, 1% yeast extract, 0.01% adenine, 0.02% tryptophan.

Capture of Protein Complexes

Strain BY4742 MATα his3Δ1 leu2Δ0 lys2Δ0 ura3Δ0 pep4Δ:KANR from the international yeast deletion consortium, or a variant strain YP2 (BY4742 pep4Δ::KANR trp1Δ:HIS3) were used for protein expression. Yeast biology techniques were essentially used as described. XY medium contains 2% bactopeptone, 1% yeast extract, 0.01% adenine, 0.02% tryptophan. To overcome difficulties in expression, such as for poorly expressed genes or developmentally regulated genes, all baits were expressed from either the inducible GAL1 or tet promoters for short induction periods. Although this approach is subject to caveat of over-expression, we minimized such effects by using short induction periods, typically 1-2 hours. The tet promoter was also used for some experiments. Other inducible systems are also generally available for this purpose. To maximize recovery of delicate protein complexes, we utilized concentrated cell extracts, from which the FLAG epitope could be captured with 50-100% efficiency (data not shown). Yeast culture volumes of 500 mL or less were used to prepare cell extracts for capture on anti-FLAG resin (Sigma-Aldrich), according to either protocol A or protocol B, as follows.

Protocols A and B were done over two physical locations.

Protocol A: BY4742 bearing pGAL1-CFLAG expressing the ORF of interest was grown in XY medium containing 2% raffinose and 0.1% glucose to an OD₆₀₀ of 1.3 to 1.5. Expression was induced with 2% galactose for 1-1.5 hours, after which cells were centrifuged and washed in lysis buffer (LB: 50 mM Hepes pH 7.5, 150 mM NaCl, 1 mM EDTA, 10 mM MgCl₂ or MgSO₄, 50 mM β-glycerophosphate, 20 mM NaF, 2 mM benzamidine, 0.5% Triton X-100, 0.5 mM DTT, 10 μg/mL leupeptin, 2 μg/mL aprotinin, 0.2 mM AEBSF, 1 mg/mL pepstatin A). The cell pellet was resuspended in 1 mL LB per gram of cells and lysed by the glass bead method. Cell extracts were clarified by centrifugation at 14,000 rpm for 20 min in a microcentrifuge. Clarified extracts were incubated with 50-80 μL of anti-FLAG-sepharose resin (Sigma-Aldrich) for 1 h at 4° C., then washed three times with wash buffer (WB; 50 mM Hepes pH 7.5, 150 mM NaCl, 1 mM EDTA, 10 mM MgCl2, 50 mM β-glycerophosphate, 5% glycerol, 0.1% TritonX-100, 0.5 mM DTT, 0.2 mM AEBSF) and once with WB without Triton X-100. To help remove background proteins, beads were then incubated for 15 min at 4° C. (referred to as the pre-elution step) in HBS (100 mM Hepes, 100 mM NaCl, 0.2 mM AEBSF) with 100 μg/mL non-specific HA competitor peptide (YPYDVPDYA, SEQ ID NO: 6, Research Genetics). FLAG-tagged protein complexes were eluted twice for 10 min. at room temperature (referred to as the elution step) in HBS with 200 μg/mL FLAG peptide (DYKDDDDK, SEQ ID NO: 3, Sigma). Eluates and pre-eluates were precipitated with TCA/deoxycholate, washed with acetone, air-dried, resuspended in protein sample buffer and were separated by SDS-PAGE on a 10-20% gradient gel (Novex). Proteins were detected by colloidal Coomassie stain (Gel-Code, Pierce) and selected for band-cutting based on their specific presence in the FLAG-tagged complex.

Protocol B: YP2 bearing ptet-CFLAG constructs were grown to near saturation, diluted to an OD₆₀₀ of 0.2 in DOB-Trp medium (QBIOgene) containing 2% glucose and 2 μg/mL doxycylin and then grown for a further 6-8 hours to a final OD₆₀₀ of 1.2-1.5. Alternatively, BY4742 bearing pGAL1-CFLAG constructs were induced as above. Capture onto anti-FLAG resin was carried out as in protocol A with the following exceptions. Cells were lysed in buffer containing 50 mM Tris pH 7.3, 150 mM NaCl, 1 mM EDTA, 10 mM MgSO₄, 50 mM β-glycerophosphate, 0.5% Triton X-100 and complete protease inhibitor cocktail (Roche). Pre-elution was carried out twice for 10 minutes at 4° C. in 50 mM Tris pH 7.3 with a mixture of Angiotensin (DDVYIHPFHL, SEQ ID NO: 7, Sigma-Aldrich) and Bradykinin (PPGFSPFR, SEQ ID NO: 8, Sigma-Aldrich) peptides at 50 μg/mL each or, alternatively, with 100 μg/mL of the peptide, YDDKDKD (Schafer-N, SEQ ID NO: 9). These peptides are quite efficient for the purpose of washing away non-specific binding polypeptides. FLAG-tagged protein complexes were eluted twice for 10 min. at room temperature in 50 mM Tris pH 7.3 with 200 μg/mL FLAG peptide (Schafer-N). All wash and elution steps were by gravity flow in 2 mL columns (Mobitech) and eluates were either precipitated with TCA as above or dried under vacuum.

Mass Spectrometry

Excised gel slices were reduced with DTT and alkylated with iodoacetamide essentially as described. In-gel digestion with porcine trypsin (Promega, Madison, Wis.) was carried out on an automated robotics system and the resulting peptides were extracted under basic and acidic conditions. Peptide mixtures were subjected to LC-MS/MS analysis on a Finnigan LCQ Deca® ion trap mass spectrometer (Thermo Finnigan, San Jose, Calif.) fitted with a Nanospray® source (MDS Proteomics), so that a much increased sample processing speed is achieved. Chromatographic separation was accomplished using a Famos® autosampler and an Ultimate® gradient system (LC Packings, San Francisco, Calif.) over Zorbax® SB-C18 reverse phase resin (Agilent, Wilmington, Del.) packed into 75 μM ID PicoFrit® columns (New Objective, Woburn, Mass.). A cluster of IBM NetFinity X330 computers were used to match MS/MS spectra against gene and protein sequence databases. Protein identifications were made from the resulting mass spectra using two commercially available search engines, Mascot® (Matrix Sciences, London, UK) and Sonar® (ProteoMetrics, Winnipeg, Canada). A relational database system called Piranha was developed to store and process raw mass spectrometric protein identifications. Overall, the sensitivity level that can be routinely achieved is about 50 fmol of protein loaded on to a gel. This benchmark takes into consideration all steps in the digestion/extraction/MS analysis protocol and not just specifically the MS portion.

A skilled artisan should readily understand that other equivalent instruments of similar function/specification, whether commercially available or user modified, can also be adapted for the purpose of practicing the instant invention.

Informatics Analysis of Data

The Finnigan LCQ spectrometers were set to analyze multiple samples at a high sample rate. When the bait protein was highly expressed, the cut band containing the bait which subsequently became the sample for the mass spectrometer contained very large amounts of bait protein. If a large amount of bait protein was present, then the protein may adhere to the column on the LCQ. The result was that the bait peptides on the column may “carry over” into subsequent samples for the mass spectrometer. This was the result of high mass spectrometer throughput coupled with high sensitivity. Steps were eventually taken to minimize or eliminate this phenomenon But in earlier data and in samples where it does appear, the “carry over” effect was accounted for as follows. Any bait protein that was identified within 10 samples (or more) following the last analyzed sample containing a bait protein was designated as “carry-over” and filtered from the data set.

When the immunoprecipitation eluates were loaded into wells on SDS-PAGE gels, eluates with very abundant amounts of bait protein on occasion would “spill over” into the adjacent lane. This spilled-over bait protein was at times identified by the mass spectrometer. If we identified a protein that was the same as a protein used as a bait on that gel and if it was loaded within 3 gel lanes on either side, we designated that protein as “spillover”, and it was filtered from the data set.

A portion of the data does not have the following proteins reported, even if they were identified by the mass spectrometer: Ssa1/2/3/4, Sse1/2, Tdh1/2/3, Asc1, Cdc19, Eft2, Eno1, Eno2, Fba1, Hsc82, Pgk1, Yef3, and ribosomal structural proteins. These proteins were found to bind promiscuously to many proteins. For a subset of the samples, these were not reported in the database for time considerations. The data is stored in its original state in the Sonar® database (ProteoMetrics, Winnipeg, Canada); the above proteins have not been excluded from the Sonar database.

Background Filtering Criteria

As a consequence of both the gentle isolation methods used to recover protein complexes from concentrated extracts and the ultra-sensitive mass spectroscopy used to identify proteins in each gel slice, we detected non-specific contaminants in each complex purification. These recurrent background species were filtered from the dataset according to the following criteria: (i) any protein found in association with 3% or more of the baits assayed; (ii) structural components of the ribosome, which were detected in virtually every preparation; (iii) all proteins that detectably bound to anti-FLAG resin in the absence of a FLAG-tagged bait protein (see Tables 4-6; excluded proteins listed in of frequency).

The Ty proteins are viral elements that are inserted in multiple places in the yeast genome. There is a distinct identifier for each one, even though they are all nearly the same (and generally indistinguishable by MS). It was decided that all Ty elements would be excluded from the filtered dataset due to their overall high frequency of identifications, even though any particular Ty protein ID may not have been reported many times. Table 6 lists all the different Ty proteins that were excluded.

One distinct advantage of the HMS-PCI approach is that non-specific interactions are more readily identified as the size of the dataset increases. An inherent difficulty with any data filtering scheme is that proteins that participate in many bona fide interactions are at risk of being excluded from analysis. Proteins of note in this category included actin, tubulin, karyopherins, chaperonins and heat shock proteins, all of which are known to form numerous distinct and biologically relevant complexes. As a specific example, many relevant interactions with replication factor A, an abundant trimeric complex involved in DNA replication and repair comprised of Rfa1, Rfa2 and Rfa3, were not included in the data set as a consequence of stringent filtering criteria (see Table 4). Application of these filtering criteria reduced the dataset to 4209 distinct protein identifications in association with 511 baits (Tables 2 and 3). In its entirety, the interaction set contains 1,841 different proteins or approximately 29% of the yeast proteome. Although, the filtering process eliminated 77% of the 18,411 putative interactions identified, it only eliminated 30% of the total unique proteins.

Filtering Proteins that Bound Just the FLAG Resin

To identify all the proteins that bound non-specifically just to the anti-FLAG resin, mock immunoprecipitations were done without the plasmid containing the FLAG-tagged protein. These were loaded on an SDS PAGE gel, and the entire lane was cut into band-size slices for analysis by mass spectrometry. This was done for both protocol A and protocol B. All the proteins found in these mock immunoprecipitations were used to exclude the same proteins identified in the data set as background. Mock immunoprecipitations done using protocol A were used to filter protocol A data, and mock protocol B immunoprecipitations were used to exclude protocol B data

Filtering of Promiscuous Binders

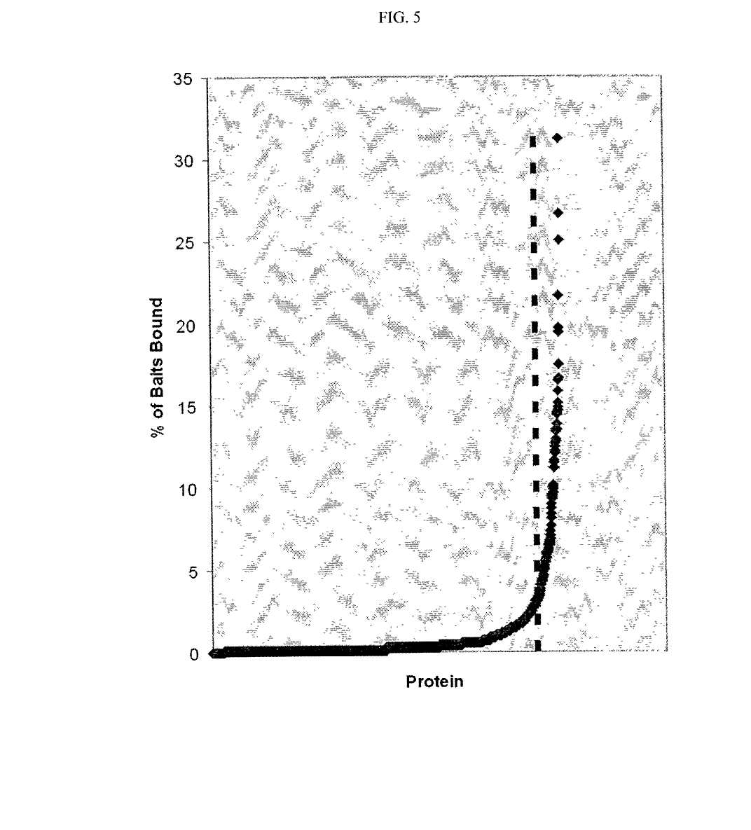

Proteins that bound to numerous bait proteins were excluded from the data set as promiscuous binders. Exclusion was based on the number of different bait proteins that a protein bound. A graph was drawn for the percentage of different bait proteins with which each identified protein associated (FIG. 5). The graph shows a distribution where above a certain percentage of baits bound by a protein, the percentage bound increases dramatically. This was then taken as the percentage of baits bound by a protein above which the protein is likely a background, promiscuous binder. The interacting proteins to the right of the dotted line in FIG. 5 were taken as background proteins because they bound many baits. This line corresponds to 3% of the total baits bound. The filter for protocol A and B was set such that any protein that bound 3% or more of the total of baits in the protocol A or B data set, respectively was filtered.

Filtering Immunoprecipitation Experiments

Immunoprecipitation experiments were excluded if any of the cut bands yielded 10 or more filtered protein identifications. These immunoprecipitations are likely technical errors that affected the “cleanliness” of the immunoprecipitation.

Analyses of Large-Scale Protein Interaction Datasets

To enable systematic comparisons of large-scale protein interaction data sets, it was necessary to develop models for representation of interaction networks. The HMS-PCI dataset was compared to two comprehensive high-throughput yeast two-hybrid (HTP-Y2H) datasets^{3,4} using interactions reported in the literature as a benchmark. An important consideration in such comparisons is that any given immunoprecipitation experiment reflects a population of protein complexes with unknown topologies, which cannot be accurately represented as pairwise protein interactions. Two models, spoke and matrix, were devised to represent these complexes as hypothetical pairwise interactions to allow comparison with HTP-Y2H pairwise protein interaction datasets. The spoke model represents the data as direct bait interactions with associated proteins as follows:

Complex: C={b, c, d, e}(b=bait; c, d, e=bait-associated proteins)

Spoke Model Interactions: iₛ={b−c, b−d, b−e}

This model does not take into account indirect interactions between bait and the associated proteins (false positives) or interactions among the associated proteins themselves (false negatives). The matrix model represents the set of bait and associated proteins as an N×N matrix, with a row and a column for each protein in the set. All possible interactions between every protein in the set are then present in the matrix entries as follows:

Complex: C={b, c, d, e}

Matrix Model Interactions: i_{M}={b−b, b−c, b−d, b−e, c−c, c−d, c−e, d−d, d−e, e−e}

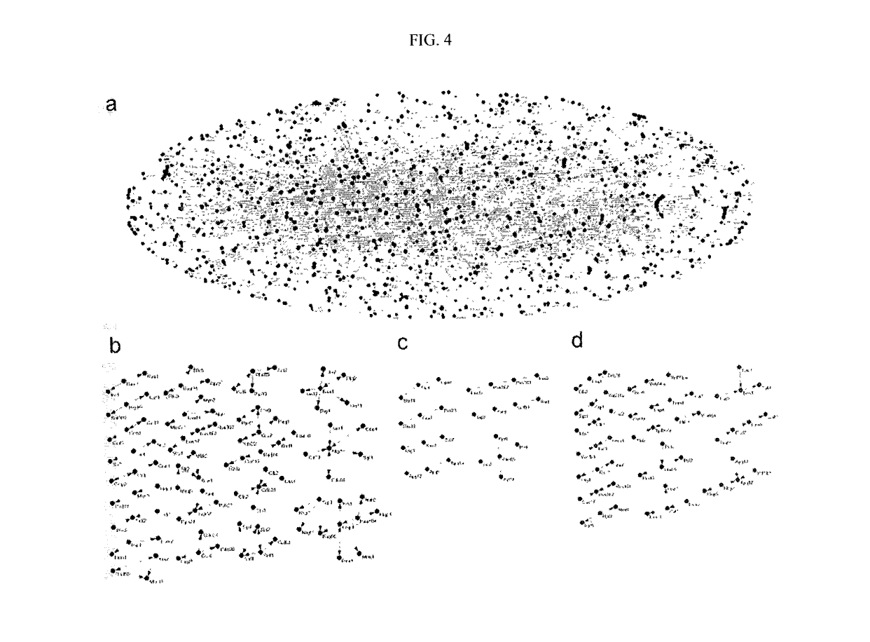

This model takes into account indirect interactions and generates many false positives (false hypothetical interactions), but no false negatives (missed real interactions). Both the spoke and matrix representations of the HMS-PCI dataset follow a power-law distribution for connectivity (FIG. 4A)⁴⁶⁻⁴⁸.

All datasets were entered into the Biomolecular Interaction Network Database (BIND), which has been designed as a standardized repository for all forms of biological interaction data, including protein-protein and genetic interactions⁴⁹. To systematically compile a set of published interactions as a benchmark, we used a search engine called PreBIND, a support vector machine (SVM) and natural language processing based algorithm used to help identify abstracts in PubMed that describe protein-protein interactions. Once a potential interaction is found by the SVM, it is vetted by an indexer and entered into BIND. Beginning with all bait proteins used in this study, PreBIND was used to collect a non-exhaustive set of 709 protein interactions from the literature. For comparison purposes, the HTP-Y2H and PreBIND datasets were normalized to correspond to baits used in this study. The spoke and matrix model representations of the HMS-PCI dataset contained approximately 3-fold greater published interactions than either of the HTP-Y2H studies (Table 3 and FIG. 4B, C). In particular, we detected 80 literature-validated interactions in the spoke model with 85 baits that failed to identify any interactions in the corresponding library-based HTP-Y2H screen³. Furthermore, over 148 common baits, an array-based HTP-Y2H screen⁴ yielded 29 validated interactions from 87 productive baits while the HMS-PCI approach generated 45 validated interactions from 121 productive baits. In addition to published interactions, a number of novel interactions were shared by the HMS-PCI and HTP-Y2H datasets (FIG. 4D).

It has been noted that the large-scale organization of metabolic networks in Archaea, Eubacteria and Eukaryotes are scale-free and follow a power law distribution for connectivity. Networks of this type are robust and error-tolerant. A similar power law distribution is also evident in HTP-Y2H interaction data sets. An analysis of the connectivity in the HTP-MS/MS network, in either the spoke or matrix representation, also revealed a power law distribution. Thus, the higher density of interactions in the HTP-MS/MS data set do not alter the overall properties of the network

Bioinformatics

All filtered interactions were entered into BIND, the Biomolecular Interaction Network Database. BIND is built around an ASN.1 specification standard that stores all relevant information about the interacting partners, including experimental evidence for the interaction, subcellular localization, biochemical function, associated cellular processes and links to the primary literature. BIND is an open source public database implemented by the Blueprint consortium and is freely available at the BIND web site. A BIND yeast import utility was developed to integrate data from SGD, RefSeq, Gene Registry, the list of essential genes from the yeast deletion consortium and GO terms. This tool ensures proper matching of any yeast gene or protein name to a protein coding region and accession number, and thereby eliminates nomenclature redundancy during import of yeast protein interaction data into BIND for visualization and analysis. Tools from the BIND project used here are written in ANSI C using the cross-platform NCBI Toolkit available at the NCBI web site. Programs were developed and run on the Linux and the Windows computer platform. Source code for the BIND database and data management system is freely available under the GNU Public License online. BIND records, tables of filtered and unfiltered protein complexes, and supplemental tables are available in electronic format at the MDS Proteomics web site.

For generation of hypothetical matrix interactions, a program called “spoke2matrix” was written to automatically convert protein complex data (i.e., the bait and associated proteins) to the matrix representation as described in the text. In instances where the same bait was used more than once, matrix interactions were generated from the results of individual immunoprecipitation experiments. A program called “common” was written to compare HMS-PCI and HTP-Y2H to literature-derived interactions detected with PreBIND. A program called “intfiltnorm” was used to normalize HMS-PCI and HTP-Y2H datasets to contain only interactions in which an interacting partner had been used as a bait in our HMS-PCI study. Interaction comparisons for overlap calculation purposes were treated as reflexive (i.e. A−B=B−A), and datasets were compiled as lists of pairwise gene names. All three programs described in this section convert an input list of yeast gene or protein name pairs to Refseq NCBI GI numbers for rapid internal processing using the BIND yeast import tool (see above).

Visualization of protein interaction networks was performed with Pajek, a program designed for large network analysis, and freely available for non-commercial use. BIND can export an arbitrary molecular interaction network as a Pajek network file. FIG. 4A was created with the Pajek program using a Fruchterman-Reingold automatic 3D layout with factor 3. Other network representations were manually constructed using Pajek. An additional program called “ip2fig” was written to create a Pajek network file with arrows pointing from bait protein to an experimentally determined associated protein and/or with previously known interactions from the PreBIND set highlighted.

The connectivity distribution of the spoke model network was calculated using the Pajek software package by partitioning the network by node (protein) degree (k). The resulting partition was exported to Microsoft Excel where the graph of the probability P(k) that a node in the network interacts with k other nodes was plotted versus k. The resulting graph could be fitted using a power-law with an R² value of 0.92. The power-law relationship was P(k)=1098 k^{-1.7297}. The fit of the connectivity distribution to this power-law was worse at higher values of k, most likely from the effects of the filter that was applied to the raw HMS-PCI data to remove background and from the fact that the spoke model does not take indirect interactions into account. Metabolic and protein interaction networks discovered so far follow a power-law connectivity distribution. Such networks are robust and maintain their integrity when subjected to random disruption of components. The distribution of the matrix model representation of the HMS-PCI dataset also followed a power-law relationship, but not as closely as the spoke model. The relationship was y=865.68×^{-1.2181} with an R² value of 0.83.

The invention also uses standard laboratory techniques, including but are not limited to recombination-based molecular cloning, yeast cell culture, immunoprecipitation, SDS-PAGE electrophoresis, protein complex isolation, in-gel protease digestion, etc. Such information can be readily found in a number of standard laboratory manuals such as Current Protocols in Cell Biology (CD-ROM Edition, ed. by Juan S. Bonifacino, Jennifer Lippincott-Schwartz, Joe B. Harford, and Kenneth M. Yamada, John Wiley & Sons, 1999).

Systematic Identification of Protein Interaction Networks in Saccharomyces cerevisiae by Mass Spectrometry

The recent deluge of genome sequence data has brought an urgent need for systematic proteomics to decipher the encoded protein networks that dictate cellular function. Here, we report a large-scale application of mass spectrometry to identify protein-protein interactions in complexes isolated from the budding yeast S. cerevisiae. Beginning with over 10% of predicted yeast proteins as baits, more than 40,000 LC-MS/MS identifications of associated proteins were made. This raw data set was filtered to render a set of 4,209 detected interactions that covered 29% of the yeast proteome. Numerous inter-pathway connections and novel multi-protein complexes were identified in various DNA damage, cell cycle and signaling pathways. Compared to previous large-scale two-hybrid studies, we achieved a 3-fold higher success rate in detecting known interactions. High-throughput mass spectrometric approaches will permit comprehensive analysis of complex proteomes, including the set of all predicted human proteins.

Mass Spectrometry

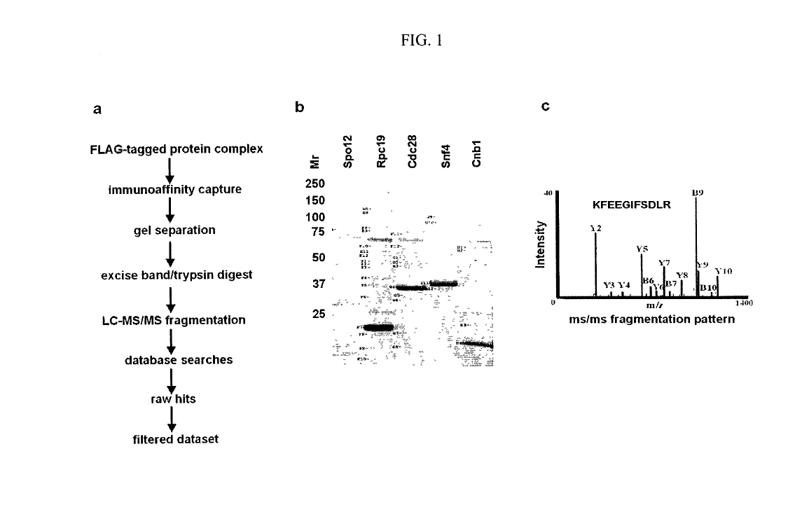

As a preliminary survey of the yeast proteome, we chose a set of 725 bait proteins that represent a variety of different functional classes, including 86 proteins implicated in DNA damage and repair, 100 protein kinases and 168 baits used in array based two hybrid screens⁴. A small scale, one-step immunoaffinity purification based on the FLAG epitope tag was used to capture protein complexes. 1,362 individual immunoprecipitations were resolved by SDS-PAGE, followed by detection of specific proteins by colloidal Coomassie stain, excision of proteins from the gel and tryptic digestion for mass spectrometric analysis (FIG. 1).

Mass spectrometric identification of proteins is achieved by comparison of peptide mass fingerprints or partial sequence information derived from peptide fragmentation patterns to gene and protein databases⁸. Our isolation procedure often yielded complex protein mixtures from single excised bands, which could not be resolved by peptide-mass-fingerprinting alone. Therefore, we used MS/MS fragmentation to unambiguously identify proteins in each band. In yeast, as in higher eukaryotes, a single MS/MS spectrum of a unique peptide is often sufficient to identify a protein. To achieve high-throughput MS/MS protein complex identification (HMS-PCI), we constructed an automated proteomics network of mass spectrometers, based on nano-HPLC-electrospray ionization-MS/MS, capable of continuous operation. On average, we generated approximately 60 MS/MS spectra per gel slice that, when matched to the protein sequence database, allowed definitive identification of proteins even in complex mixtures. 15,683 gel slices were processed, yielding approximately 940,000 MS/MS spectra that matched sequences in the protein sequence database (Table 1). 40,527 protein identifications were made in total, corresponding to 18,411 potential interactions with the set of bait proteins (Table 1). An average of 3.1 proteins were identified per excised band. This raw dataset was filtered according to empirically derived criteria to yield 4,209 distinct proteins in association with 511 baits (Table 1). The filtered interaction set contains 1,841 different proteins representing 29% of the yeast proteome (Table 2; see also MDS Proteomics web site). Of the proteins identified, 734 corresponded to previously undocumented proteins predicted from the yeast genome sequence. Additional complexes identified subsequently are listed in Table 8.

Validation of HMS-PCI

The HMS-PCI approach was validated in part by detection of known complexes from a variety of subcellular compartments (Table 2). For example, we recovered all major components of the Arp2/3 complex that nucleates actin polymerization in the cytoplasm, including Arp2, Arp3, Arc15, Arc18, Arc19, Arc 35 and Arc40⁹. Similarly, the eIF2 translation initiation complex, composed of Sui2/3, Gcd1/2/6/11 and Gcn3, was recovered with a Sui2 bait¹⁰. A number of transcription factor complexes were recovered, including the Met4 complex that regulates methionine biosynthesis gene expression. Notably, Met4 was detected in conjunction with the SCF^{Met30} ubiquitin ligase components Met30, Cdc53, Skp1, Hrt1 and Rub1, which negatively regulate Met4, as well as with its transcriptional co-regulator Met31¹¹. We were similarly able to capture and identify multi-protein complexes in the vesicular (e.g., Vps21, Ypt1, Cop1), nucleolar (e.g., Nop13, Ygr103w) and membrane (e.g., Ras2, Yck1/2, Kin2, Kre6) compartments. Below we describe a limited subset of the numerous interactions detected by HMS-PCI, which illustrate the ability of this approach to discover protein function and to identify inter-pathway connections.

Phosphorylation-based Signaling Complexes

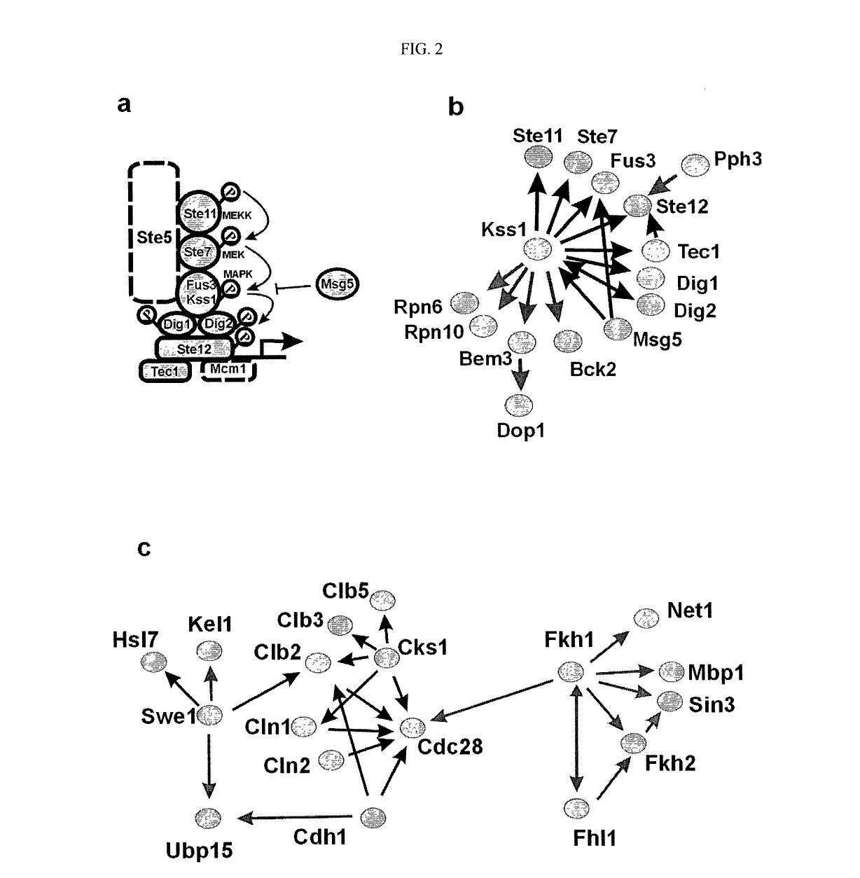

As protein phosphorylation underlies many cellular signaling events, the identification of biologically relevant substrates and regulators for kinases and phosphatases is crucial for a global understanding of cell regulation¹. To approach this issue from a proteome-wide perspective, we used 100 of the 122 kinases encoded by the yeast genome, as well as 36 phosphatases and phosphatase regulatory subunits, as baits to capture associated signaling components (Table 2). As an example, we recovered numerous known and novel interactions with several mitogen activated protein kinases (MAPKs). In haploid cells, the mating pheromone/filamentous growth signal is transmitted by the archetypal MAPK module, Ste11/Ste7/Fus3/Kss1, in a response that has been under intense genetic and biochemical scrutiny for nearly 30 years¹². HMS-PCI analysis of complexes captured with Kss1 identified many known components of the pathway, including Ste11, Ste7, and four known downstream targets, the transcriptional regulators, Ste12, Tec1, Dig1/Rst1, and Dig2/Rst2 (FIG. 2A, B). In addition, we identified other novel Kss1 interactions of potential biological significance. Bem3 is a GTPase activating protein that may be recruited to Kss1 signaling complexes in order to attenuate the Cdc42 Rho-type GTPase, an upstream activator of the pathway¹³. Bck2 is an activator of the G1/S transcriptional program that may be targeted by Kss1 during pheromone induced G1 arrest; indeed, a bck2 mutant is hypersensitive to mating pheromone, while overexpression of BCK2 causes pheromone resistance¹⁴. Biologically relevant interactions were also detected with other MAPKs, including between the cell wall integrity MAPK Slt2 and its upstream activators Bck1 and Mkk2¹², and between the osmotic stress response MAPK Hog1 and a downstream target kinase, Rck2¹⁵. Consistent with its genetic role in attenuating the pheromone and cell wall integrity responses^{16,17}, the dual specificity phosphatase Msg5 was associated with Fus3, Kss1 and Slt2 (Table 2).

Numerous proteins were detected in association with Cdc28, a cyclin dependent kinase that controls many aspects of cell division (FIG. 2C). We identified interactions between Cdc28 and its regulatory partners Cks1, an essential tight binding subunit, and the cyclins Cln1, Cln2, Clb2, Clb3 and Clb5 (ref. 18). Probable upstream and downstream connections to Cdc28 were also found. The dual-specificity kinase Swe1, which mediates the morphogenesis checkpoint arrest via inhibitory phosphorylation of Cdc28, was associated both with Clb2 and Hsl7, a known negative regulator of Swe1 (ref 19). A novel interaction between Swe1 and Kel1, a protein that is involved in cell fusion and cell polarity²⁰, might signal the establishment of polarized growth to Swe1. Numerous events in mitosis are activated by Clb1/2-Cdc28, including a transcriptional positive feedback loop that controls expression of CLB1/2 and other G2/M regulated genes, via the forkhead transcription factors, Fkh1 and Fkh2²¹. Cdc28 was detected in association with Fkh1, providing direct physical closure of the kinase-transcription factor circuit. In addition, Fkh1, Fkh2 and a related forkhead transcription factor Fhl1 were found in complex with one another. Fhl1 has not yet been implicated in G2/M transcriptional control, but given that a fkh1 fkh2 double mutant is viable, it is possible that Fhl1 contributes to transcriptional activation in the absence of Fkh1/2. Intriguingly, Fkh1 interacted with Net1, a nucleolar protein required for rDNA silencing and mitotic exit, and both Fhl1 and Net1 are required for proper Poll-dependent expression of rDNA genes^{22,23}. Furthermore, both Fkh1 and Fkh2 associated with Sin3, a component of the histone deacetylase machinery that represses many genes²⁴, consistent with the postulated role of Fkh1/2 as transcriptional repressors in other phases of the cell cycle²¹.

A recently discovered cell cycle pathway called the Mitotic Exit Network (MEN) is based on the protein kinases Cdc5, Cdc15, Dbf2 and Dbf20, the protein phosphatase Cdc14, and other proteins²⁵. The polo domain-containing kinase Cdc5 was found in association with the cohesin complex, composed of Smc1, Smc3, Mcd1/Scc1 and Irr1 (Table 2). These interactions corroborate the recent finding that Cdc5 can phosphorylate the Mcd1/Scc1 subunit of cohesin to promote sister chromatid separation²⁶. A novel interaction with the spindle pole body (SPB) protein Spc72 probably reflects localization of Cdc5 and other MEN components to the SPB in early M phase^{27,28}. HMS-PCI also revealed connections between MEN components themselves, including Dbf2-Mob1, Dbf20-Mob1, Tem1-Bfa1, Tem1-Cdc15, as well as several novel interactions (Table 2).

Many protein kinases and phosphatases are regulated by tight binding subunits, which serve to localize or control activity¹. We identified several known examples of interactions between kinases and inhibitory subunits, such as between the Tpk1/2/3 cAMP-dependent protein kinases and the regulatory subunit Bcy1, as well as between several cyclins and their cognate Cdk partners. The type 1 protein phosphatase catalytic subunit Glc7 regulates a variety of cellular processes by association with at least 6 different regulatory subunits, of which we identified 4 (Sds22, Reg1, Gip2, Glc8). Other novel interactions detected with Glc7 suggested a role in chromosome segregation and cell cycle (Cdc14, Ytm1, and Ygr103w), glycogen metabolism (Gph1), cell fusion and polarity (Kel1) and RNA processing (Fip1, Cft1 and Sen1). In other examples, we detected the regulatory subunits Cdc55, Rts1, Tpd3 and Tap42 in association with the PP2 phosphatases, Pph21 or Pph22. A protein of unknown function that is induced in response to DNA damage, Ygr161c, bound to both Pph21 and Pph22 and may represent a novel regulatory subunit. Another unknown, Ydr071c, interacted with the type PP2C phosphatases, Ptc3 and Ptc4. Taken together, the above examples demonstrate that HMS-PCI can readily chart protein complexes in phosphorylation-based signaling networks.

A Cellular Network—The DNA Damage Response

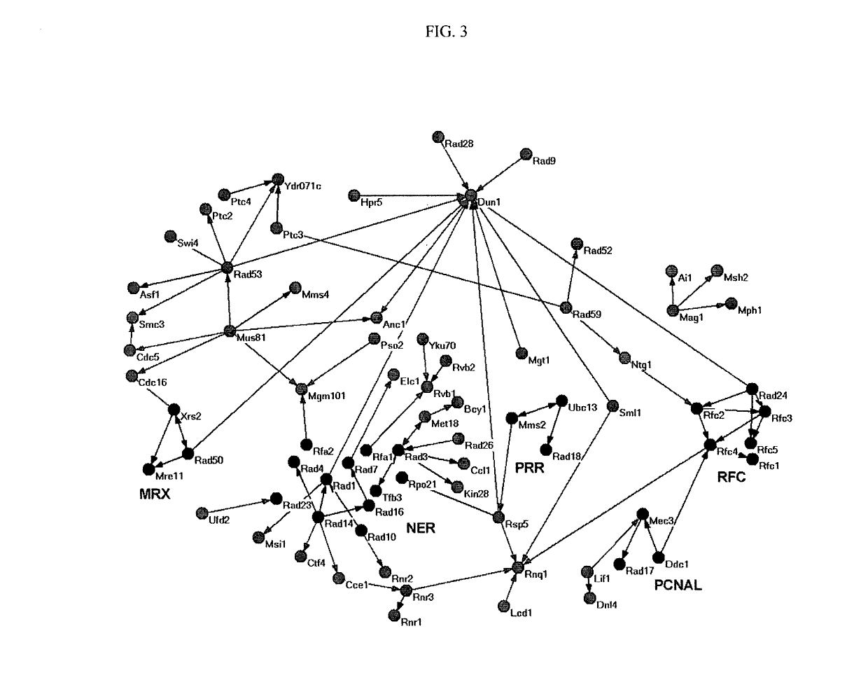

To test the ability of HMS-PCI to identify new connections and components in an entire biological process, we analyzed protein complexes centered on 86 proteins known to participate in the DNA Damage Response (DDR) in yeast. The DDR is critical for maintenance of genome stability and depends both on numerous DNA repair processes and on signaling cascades, called checkpoint pathways, that control cell cycle progression, transcription, apoptosis, protein degradation and the DNA repair pathways themselves²⁹. The global DDR network revealed by HMS-PCI is not only highly enriched in known interactions but also contains many novel interactions of likely biological significance (FIG. 3). Examples of known interactions include: the replication factor C complex (RFC, Rfc1-5) and the RFC^{Rad24} subcomplex, as well as the PCNA-like (PCNAL) Mec3/Rad17/Ddc1 complex, both of which transduce DNA damage signals; part of the Mms2/Ubc13/Rad18 post-replicative repair (PRR) complex; and the Mre11/Rad50/Xrs2 (MRX) complex that mediates double strand break repair by homologous and non-homologous mechanisms²⁹. Although the small scale immunoprecipitations we used rarely yielded complete complexes, the comprehensive coverage of DDR proteins readily identified pathway and network connections. For example, we recovered Rfc4 in Ddc1 complexes, consistent with the hypothesis that the PCNAL complex might be loaded onto DNA by the RFC^{Rad24} complex³⁰. Our analysis of nucleotide excision repair (NER) proteins revealed the extensive network of interactions in this process (Table 2, FIG. 3). We recovered nearly all known nucleotide excision repair (NER) factors in their dedicated subcomplexes³¹: Rad1-Rad10-Rad14 (NEF1); Rad3-TFB3-Kin28-Ccl1 (NEF3/FFIIH) and Rad7-Rad16 (NEF4). The Rad4-Rad23 interaction (NEF2) was not found, but we nevertheless detected an association between Rad4 and NEF1, a known interaction among NER factors. In addition to these previously described interactions, the HMS-PCI approach unraveled novel interactions of interest in almost all aspects of the DDR, a few of which are presented below.

The Rad53 protein kinase is a central transducer of DNA damage²⁹ and is the yeast orthologue of Chk2, the product of the gene mutated in the cancer syndrome variant Li-Fraumeni³². HMS-PCI analysis confirmed the known Rad53 interaction with Asfl^{33,34} and yielded several novel complexes of likely biological significance. Rad53 captured the PP2C-type phosphatase Ptc2, which is genetically implicated as a negative regulator of RAD53-dependent DNA damage signalling³⁵. Furthermore, the uncharacterized gene product Ydr071c was detected with both Rad53 and the PP2C family members, Ptc3 and Ptc4, suggesting that Ydr071c may be a DDR-specific regulatory factor of PP2C-type phosphatases. Consistent with this physical interaction, we find a genetic interaction between YDR071C and RAD53 (R Woolstencroft and D. D., unpublished). With regard to Rad53 substrates, the putative targets Swi4 (ref. 36)and Cdc5 (ref 37) were directly or indirectly connected to Rad53 by HMS-PCI.

The Dun1 protein kinase has a similar overall structure to Rad53 and Chk2, most notably the presence of a phosphothreonine-binding module termed the FHA domain³⁸. The HMS-PCI interaction profile of Dun1 included the potential upstream regulators Rad9, Rad53, Rad24, Hpr5 (Srs2) and Rad50. Of particular note is the interaction with Sml1, an inhibitor of ribonucleotide reductase that is phosphorylated in a DUN1-dependent manner, an event proposed to target Sml1 for degradation³⁹. Dun1 also interacted with Rsp5, an E3 ubiquitin ligase reported to target the RNA polymerase II large subunit (Rpo21) for ubiquitin-mediated degradation following DNA damage⁴⁰. Rsp5 is thus a candidate for the E3 enzyme that targets Sml1 for degradation after DNA damage.

Despite being one of the best understood DNA repair processes, some aspects of excision repair are still poorly defined. For example, the biochemical function of Met18/Mms19 has been particularly elusive³¹. The HMS-PCI approach revealed that Met18 can interact with Rad3, a component of the TFIIH complex needed for both RNA PolII-dependent transcription and NER A further regulatory connection is suggested by our detection of an association between Met18 and Bcy1, the regulatory subunit of the yeast cyclic AMP-dependent kinases. As deletion of BCY1 causes ultraviolet (UV) radiation resistance⁴¹, it is possible that Met18 links the PKA pathway to the NER machinery via its dual interaction with Bcy1 and TFIIH. Further links between excision repair and the ubiquitin system were revealed by analysis of Rad23, which contains a ubiquitin-like (UBL) domain, two ubiquitin-associated (UBA) domains and a unique region that binds Rad4 (ref. 31). The interaction detected between Rad23 and the ubiquitin chain assembly factor Ufd2 (ref. 42) is corroborated by genetic interactions that suggest RAD23 and UFD2 act antagonistically⁴³. The Rad23-Ufd2 interaction may be mediated via the UBL domain since Ufd2 also interacted with another UBL-containing protein, Dsk2. We also identified an interaction between Rad1 and Msi1, a component of the yeast chromatin assembly complex⁴⁴. Because deletion of MSI1 specifically causes UV sensitivity, the Msi1-Rad1 interaction suggests a means by which the chromatin assembly complex is recruited to UV-damaged DNA during NER.

Protein interaction data often suggests function, particularly when combined with protein sequence analysis. For example, we found that Rad7 interacts with the yeast elongin C homolog, Elc1, for which a function remains to be assigned. In mammalian cells, Elongin C associates with Elongin B, the cullin Cul2, the RING-H2 domain protein Rbx1 and any one of a number of substrate recruitment factors called SOCS-box proteins to form E3 enzyme complexes that mediate substrate ubiquitination⁴⁵. Consistent with the Elc1-Rad7 interaction, sequence alignments revealed a divergent SOCS box motif in Rad7 (A. Willems and M. T., unpublished data). Rad7 may thus be part of an E3 enzyme complex that acts during excision repair.

Identification of Hypothetical Proteins

As a byproduct of HMS-PCI, we identified many proteins of unknown function whose existence had previously only been predicted from the genome sequence. Given the difficulty in prediction of coding regions from genome sequence information even in yeast, the direct identification of encoded peptides by mass spectrometry provides an important validation of putative coding regions. Table 7 contains a list of 734 proteins identified by mass spectrometry that fall into MIPS categories other than known proteins. Tables of hypothetical and putative proteins were obtained from the MIPS (Munich Information center for Protein Sequences) classification of ORFs from the MIPS web site.

Bioinformatics Elaboration of Protein Interactions

Even when unknown proteins do not fall within obvious large networks, protein interaction data often suggests function, particularly when combined with protein sequence analysis. For example, we found that Rad7 interacts with the yeast elongin C homolog, Elc1, for which a function remains to be assigned. In mammalian cells, Elongin C associates with Elongin B, the cullin Cul2, the RING-H2 domain protein Rbx1 and any one of a number of substrate recruitment factors called SOCS-box proteins to form E3 enzyme complexes that mediate substrate ubiquitination. Consistent with the Elc1-Rad7 interaction, sequence alignments revealed a divergent SOCS box motif in Rad7. Rad7 may thus part of an E3 enzyme complex that acts during excision repair.

In another example leveraged by bioinformatics analysis, we identified a hypothetical interaction network that contains an unusually large number of redox proteins associated with isoforms of Old Yellow Enzyme (OYE), Oye2 and Oye3. OYE was the first flavoenzyme purified, but despite extensive biochemical characterization of its NADPH oxidase activity, its true function is unknown. We identified 14 oxidoreductases of diverse functions in association with OYE isoforms, including Adh1, Rnr4, Sod1, Erg27 and Tyr1. An intriguing possibility is that OYE supplies oxidoreductase activity by channeling reducing equivalents to other oxidoreductases and their substrates, as mediated through specific protein-protein interactions.

Finally, it is likely that all protein complexes must be interconnected in order to allow coordination of diverse cellular functions. Such interactions should be readily revealed by non-directed, proteome-wide analysis. In one striking instance, we uncovered a large, previously undescribed network of interactions between proteins that are either localized to the nucleolus or involved in rRNA processing. One element of the network is formed by proteins of the U3 snoRNP complex, as revealed by interactions spanning several different baits. Similarly, the presence of several MEN proteins at the periphery of this network is consistent with the nucleolar sequestration of Cdc14 by Net1, and the role of Net1 in rDNA transcription. By virtue of their connections to the network, three proteins of unknown function, Ykr081c, Ylr427w and Yhr052w are implicated in nucleolar processing or regulation.

Prospects

The ultimate utility of any large scale platform rests upon its ability to reliably glean new insights into biological function. The instant invention provides the first high-throughput analysis of native protein complexes by highly sensitive mass spectrometric identification methods HMS-PCI. Importantly, proteome-wide analysis allows the detection of complex cellular networks that might otherwise elude more focused approaches. The numerous interconnections revealed in this study suggests that only a fraction of proteins need be investigated to obtain near complete coverage of the proteome. For example, linear extrapolation suggests that interactions captured with 2,500 bait proteins should connect the entire yeast proteome. Given that approximately 40% of yeast proteins are conserved through eukaryotic evolution⁵⁰, the global yeast protein interaction map will provide a partial framework for understanding the human proteome. Imminent technical advances, such as the direct analysis of protein complexes without electrophoretic separation, as well as even higher sensitivity mass spectrometers, will undoubtedly extend the reach of the approach described here. Given that the set of proteins nominally encoded by the human genome is only 5-fold greater than the total number of yeast proteins, comprehensive analysis of the human proteome is feasible with current technology.

Methods

Recombination-based cloning, yeast culture and isolation of protein complexes were carried out using standard methods and are described above. Protein bands visualized by colloidal Coomassie stain were excised from polyacrylamide gels, reduced and S-alkylated, then subject to trypsin hydrolysis^{51,52}. LC-MS/MS analysis was performed on a Finnigan LCQ Deca® ion trap mass spectrometer (Thermo Finnigan, San Jose, Calif.) fitted with a Nanospray® source (MDS Proteomics). Chromatographic separation was via a Famos® autosampler and an Ultimate® gradient system (LC Packings, San Francisco, Calif.) over Zorbax® SB-C18 reverse phase resin (Agilent, Wilmington, Del.) packed into 75 μM ID PicoFrit® columns (New Objective, Woburn, Mass.). Protein identifications were made from the resulting mass spectra using the commercially available search engines Mascot® (Matrix Sciences, London, UK), Sonar® (ProteoMetrics, Winnipeg, Canada) and Sequest® (ThermoFinnigan, San Jose, Calif.). Both the raw and filtered datasets generated in this study are available at the MDS Proteomics web site. The filtered dataset has been deposited in BIND⁴⁹ and can be viewed at the BIND web site.

### REFERENCE

1. Pawson, T. & Nash, P. Protein-protein interactions define specificity in signal transduction. Genes Dev. 14, 1027-1047 (2000).

2. Fields, S. & Song, O. A novel genetic system to detect protein-protein interactions. Nature 340, 245-246 (1989).

3. Ito, T. et al. A comprehensive two-hybrid analysis to explore the yeast protein interactome. Proc. Natl. Acad. Sci. USA 98, 4569-4574 (2001).

4. Uetz, P. et al. A comprehensive analysis of protein-protein interactions in Saccharomyces cerevisiae. Nature 403, 623-627 (2000).

5. Uetz, P. & Hughes, R. E. Systematic and large-scale two-hybrid screens. Curr. Opin. Microbiol 3, 303-308 (2000).

6. Lamond, A. & Mann, M. Cell Biology and the Genome Projects—a concerted strategy for characterizing multi-protein complexes using mass spectrometry. Trends Cell Biol. 7, 139-142 (1997).

7. Neubauer, G. et al. Identification of the proteins of the yeast U1 small nuclear ribonucleoprotein complex by mass spectrometry. Proc. Natl. Acad. Sci. USA 94, 385-390 (1997).

8. Mann, M., Hendrickson, R. C. & Pandey, A. Analysis of proteins and proteomes by mass spectrometry. Annu. Rev. Biochem. 10, 437-473 (2001).

9. Winter, D., Podtelejnikov, A. V., Mann, M. & Li, R. The complex containing actin-related proteins Arp2 and Arp3 is required for motility and integrity of yeast actin patches. Curr. Biol. 7, 519-529 (1997).

10. Pestova, T. V. et al. Molecular mechanisms of translation initiation in eukaryotes. Proc. Natl. Acad. Sci. USA 98, 7029-7036 (2001).

11. Patton, E. E. et al. Cdc53 is a scaffold protein for multiple Cdc34/Skp1/F-box protein complexes that regulate cell division and methionine biosynthesis in yeast. Genes Dev. 12, 692-705 (1998).

12. Gustin, M. C., Albertyn, J., Alexander, M. & Davenport, K. MAP kinase pathways in the yeast Saccharomyces cerevisiae. Microbiol Mol Biol. Rev. 62, 1264-1300 (1998).

13. Zheng, Y., Cerione, R. & Bender, A. Control of the yeast bud-site assembly GTPase Cdc42. Catalysis of guanine nucleotide exchange by Cdc24 and stimulation of GTPase activity by Bem3. J Biol Chem 269, 2369-2372 (1994).

14. Wijnen, H. & Futcher, A. B. Genetic analysis of the shared role of CLN3 and BCK2 at the G1-S transition in Saccharomyces cerevisiae. Genetics 153, 1131-1143 (1999).

15. Bilsland-Marchesan, E., Arino, J., Saito, H., Sunnerhagen, P. & Posas, F. Rck2 kinase is a substrate for the osmotic stress-activated mitogen-activated protein kinase Hog1. Mol. Cell. Biol. 20, 3887-3895 (2000).

16. Doi, K. et al. MSG5, a novel protein phosphatase promotes adaption to pheromone response in S. cerevisiae. EMBO J. 13, 61-70 (1994).

17. Watanabe, Y., Irie, K. & Matsumoto, K. Yeast RLM1 encodes a serum response factor-like protein that may function downstream of the Mpk1 (Slt2) mitogen-activated protein kinase pathway. Mol. Cell. Biol. 15, 5740-5749 (1995).

18. Morgan, D. O. Cyclin-dependent kinases: engines, clocks, and microprocessors. Annu. Rev. Cell. Dev. Biol. 13, 261-291 (1997).

19. McMillan, J. N. et al. The morphogenesis checkpoint in Saccharomyces cerevisiae: cell cycle control of Swe1p degradation by Hsl1p and Hsl7p. Mol. Cell. Biol. 19, 6929-6939 (1999).

20. Philips, J. & Herskowitz, I. Identification of Kel1p, a kelch domain-containing protein involved in cell fusion and morphology in Saccharomyces cerevisiae. J. Cell. Biol. 143, 375-389 (1998).

21. Jorgensen, P. & Tyers, M. The forked path to mitosis. Genome Biol. 1 (2000).

22. Hermann-Le Denmat, S., Werner, M., Sentenac, A. & Thuriaux, P. Suppression of yeast RNA polymerase III mutations by FHL1, a gene coding for a fork-head protein involved in rRNA processing. Mol. Cell. Biol. 14, 2905-2913 (1994).

23. Shou, W. et al. Net1 stimulates Rna polymerase I transcription and regulates nucleolar structure independently of controlling mitotic exit. Mol. Cell 8,45-55 (2001).

24. Bernstein, B. E., Tong, J. K. & Schreiber, S. L. Genomewide studies of histone deacetylase function in yeast. Proc. Natl. Acad. Sci. USA 97, 13708-13713 (2000).

25. Morgan, D. O. Regulation of the APC and the exit from mitosis. Nat. Cell. Bio.l 1, E47-53 (1999).

26. Alexandru, G., Uhlmann, F., Mechtler, K., Poupart, M. & Nasmyth, K Phosphorylation of the cohesin subunit Scc1 by Polo/Cdc5 kinase regulates sister chromatid separation in yeast. Cell 105, 459472 (2001).

27. Knop, M. & Schiebel, E. Receptors determine the cellular localization of a gamma-tubulin complex and thereby the site of microtubule formation. EMBO J. 17, 3952-3967 (1998).

28. Song, S., Grenfell, T. Z., Garfield, S., Erikson, R. L. & Lee, K. S. Essential function of the polo box of Cdc5 in subcellular localization and induction of cytokinetic structures. Mol. Cell Biol. 20, 286-298 (2000).

29. Zhou, B. B. & Elledge, S. J. The DNA damage response: putting checkpoints in perspective. Nature 408, 433-439 (2000).

30. Thelen, M. P., Venclovas, C. & Fidelis, K. A sliding clamp model for the Rad1 family of cell cycle checkpoint proteins. Cell 96, 769-770 (1999).

31. Prakash, S. & Prakash, L. Nucleotide excision repair in yeast. Mutat. Res. 451, 13-24 (2000).

32. Bell, D. W. et al. Heterozygous germ line hCHK2 mutations in Li-Fraumeni syndrome. Science 286, 2528-2531 (1999).

33. Emili, A., Schieltz, D. M., Yates, J. R. & Hartwell, L. H. Dynamic interaction of DNA damage checkpoint protein Rad53 with chromatin assembly factor Asf1. Mol. Cell 7, 13-20 (2001).

34. Hu, F., Alcasabas, A. A. & Elledge, S. J. Asf1 links Rad53 to control of chromatin assembly. Genes Dev. 15, 1061-1066 (2001).

35. Marsolier, M. C., Roussel, P., Leroy, C. & Mann, C. Involvement of the PP2C-like phosphatase Ptc2p in the DNA checkpoint pathways of Saccharomyces cerevisiae. Genetics 154, 1523-1532 (2000).

36. Sidorova, J. M. & Breeden, L. L. Rad53-dependent phosphorylation of Swi6 and down-regulation of CLN1 and CLN2 transcription occur in response to DNA damage in Saccharomyces cerevisiae. Genes Dev. 11, 3032-3045 (1997).

37. Sanchez, Y. et al. Control of the DNA damage checkpoint by Chk1 and Rad53 protein kinases through distinct mechanisms. Science 286, 1166-1171 (1999).

38. Durocher, D., Henckel, J., Fersht, A. R & Jackson, S. P. The FHA domain is a modular phosphopeptide recognition motif. Mol. Cell 4, 387-394 (1999).

39. Zhao, X, Chabes, A., Domkin, V., Thelander, L. & Rothstein, R. The ribonucleotide reductase inhibitor Sml1 is anew target of the Mec1/Rad53 kinase cascade during growth and in response to DNA damage. EMBO J. 20, 3544-3553 (2001).

40. Beaudenon, S. L., Huacani, M. R., Wang, G., McDonnell, D. P. & Huibregtse, J. M. Rsp5 ubiquitin-protein ligase mediates DNA damage-induced degradation of the large subunit of RNA polymerase II in Saccharomyces cerevisiae. Mol. Cell. Biol. 19, 6972-6979 (1999).

41. Engelberg, D., Klein, C., Martinetto, H., Struhl, K. & Karin, M. The UV response involving the Ras signaling pathway and AP-1 transcription factors is conserved between yeast and mammals. Cell 77, 381-390 (1994).

42. Koegl, M. et al. A novel ubiquitination factor, E4, is involved in multiubiquitin chain assembly. Cell 96, 635-644 (1999).

43. Ortolan, T. G. et al. The DNA repair protein Rad23 is a negative regulator of multi-ubiquitin chain assembly. Nat. Cell. Biol. 2, 601-608 (2000).

44. Kaufman, P. D., Kobayashi, R. & Stillman, B. Ultraviolet radiation sensitivity and reduction of telomeric silencing in Saccharomyces cerevisiae cells lacking chromatin assembly factor-I. Genes. Dev. 11, 345-357 (1997).

45. Tyers, M. & Rottapel, R. VHL: a very hip ligase. Proc. Natl. Acad. Sci. USA 96, 12230-12232 (1999).

46. Barabasi, A. L. & Albert, R. Emergence of scaling in random networks. Science 286, 509-512. (1999).

47. Jeong, H., Mason, S. P., Barabasi, A. L. & Oltvai, Z. N. Lethality and centrality in protein networks. Nature 411, 41-42 (2001).

48. Wagner, A. & Fell, D. A. The small world inside large metabolic networks. Proc. R. Soc. Lond. B Biol. Sci. 268, 1803-1810 (2001).

49. Bader, G. et al. BIND—The Biomolecular Interaction Network Database. Nucl. Acids Res. 29, 242-245 (2001).

50. Chervitz, S. A. et al. Comparison of the complete protein sets of worm and yeast: orthology and divergence. Science 282, 2022-2028 (1998).

51. Shevchenko, A., Wilm, M., Vorm, O. & Mann, M. Mass spectrometric sequencing of proteins silver-stained polyacrylamide gels. Anal. Chem. 68, 850-858 (1996).

52. Wilm, M. et al. Femtomole sequencing of proteins from polyacrylamide gels by nano-electrospray mass spectrometry. Nature 379, 466-469 (1996).

53. Mewes, H. W. et al. MIPS: a database for genomes and protein sequences. Nucl. Acids Res. 28, 3740 (2000).

54. Belli, G., Gari, E., Piedrafita, L., Aldea, M. & Herrero, E. An activator/repressor dual system allows tight tetracycline-regulated gene expression in budding yeast. Nucl. Acids Res. 15, 942-947 (1998).

55. Winzeler, E. A, et al. Functional Characterization of S. cerevisiae Genome by Gene Deletion and Parallel Analysis. Science 285, 901-906 (1999).

56. Guthrie, C. & Fink, G. R. Guide to Yeast Genetics and Molecular Biology. Meth. Enzymol. 194 (1991).

57. Shevchenko, A., Wilm, M., Vorm, O. & Mann, M. Mass spectrometric sequencing of proteins silver-stained polyacrylamide gels. Anal. Chem. 68, 850-858 (1996).

58. Wilm, M. et al. Femtomole sequencing of proteins from polyacrylamide gels by nano-electrospray mass spectrometry. Nature 379, 466-469 (1996).

59. Bader, G. & Hogue, C. BIND—a data specification for storing and describing biomolecular interactions, molecular complexes and pathways. Bioinformatics 16,465-477 (2000).

60. Chervitz, S. A. et al. Using the Saccharomyces Genome Database (SGD) for analysis of protein similarities and structure. Nucl. Acids Res. 27, 74-78 (1999).

61. Pruitt, K. D. & Maglott, D. R. RefSeq and LocusLink: NCBI gene-centered resources. Nuc. Acids Res. 29, 137-140 (2001).

62. Ashburner, M. et al. Gene ontology: tool for the unification of biology. The Gene Ontology Consortium. Nat. Genet. 25, 25-29 (2000).

63. Batagelj, V. & Mrvar, A. Peek—Program for large netwrk analysis. Connections 2, 47-57 (1998).

64. Jeong, H., Tombor, B., Albert, R., Oltvai, Z. N. & Barabasi, A. L. The large-scale organization of metabolic networks. Nature 407, 651-654 (2000).

65. Jeong, H., Mason, S. P., Barabasi, A. L. & Oltvai, Z. N. Lethality and centrality in protein networks. Nature 411, 4142 (2001).

66. Wagner, A. & Fell, D. A. The small world inside large metabolic networks. Proc. R. Soc. Lond B Biol. Sci. 268, 1803-1810 (2001).

67. Barabasi, A. L. & Albert, R. Emergence of scaling in random networks. Science 286, 509-512. (1999).

68. Albert, R., Jeong, H. & Barabasi, A. L. Error and attack tolerance of complex networks. Nature 406, 378-382 (2000).

69. Wagner, A. Robustness against mutations in genetic networks of yeast. Nat. Genet. 24, 355-361 (2000).

All cited references, patents, publications are hereby incorporated by reference.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

TABLE 1
Summary of HMS-PCI analysis

| | |
|---|---|
| BEFORE | AFTER |
| FILTERING | FILTERING |

| | |
|---|---|
| NUMBER OF | 1368 |
| IMMUNOPRECIPITATON | |
| EXPERIMENTS | |
| NUMBER OF BAIT | 724 |
| PROTEINS ATTEMPTED | |
| NUMBER OF BAITS | 605 |
| ASSAYED WHERE BAIT | |
| PROTEIN WAS | |
| IDENTIFIED BY MS¹ | |
| NUMBER OF BAITS | 511 |
| ASSAYED WITH AT | |
| LEAST 1 COMPLEX | |
| INTERACTOR AFTER | |
| FILTERING | |
| MS IDENTIFICATIONS | 42,275 |

| | | |
|---|---|---|
| IDENTIFIED COMPLEX | 19,085 | 4,171 |
| INTERACTIONS WITH | | |
| THE BAIT PROTEIN | | |
| UNIQUE PROTEINS IN | 2,604 | 1,821 |
| DATASET | (42% OF | (29% OF |
| | GENOME) | GENOME) |

¹proteins of less than 20 kDa were assumed to have run off SDS-PAGE gels.

TABLE 2
Protein complexes detected by HMS-PCI.

| BAIT | ASSOCIATED PROTEINS |
|---|---|
| AATI | MGM1, YJL204C |
| ABP1 | ARP3, HXT7, MPS1, SCP1, SPS1, TUP1, YSC84 |
| AFG3 | ENP1, GCD6, IMD2, LPD1, MET3, YHR113W |
| AIP1 | IMH1 |
| AKL1 | HTS1 |
| APG12 | AAC1, AAC3, ADE5, 7, APG17, ARC1, ARO1, ARP2, |
| | CAR2, CPA2, CPR6, CRM1, CVT9, FET3, FET4, |
| | GCD11, GFA1, IPP1, KAP122, KGD1, MET10, MET18, |
| | PFK1, PPX1, PRB1, REP1, REX2, RPN1, RPN10, RPN11, |
| | RPN3, RPN5, RPN6, RPN7, RPT1, RPT3, SEC18, TIF2, |
| | TYR1, YDR214W, YGL245W, YHR020W, YHR033W, |
| | YHR076W, YNL208W, YOR086C |
| APG5 | CYS3, FET3, HST1, MOT1, PDR13, STI1, TSL1 |
| APM1 | APL2, APL4, BFR2, BRX1, CBR1, HEM15, KRE33, |
| | KRE6, KRI1, KRR1, MDJ1, MGM101, NOG1, NOP4, |
| | PAB1, PBP1, PWP1, RCL1, RPB5, RPN1, SEC28, SEC6, |
| | SIK1, SNF4, THS1, TIF6, TUF1, UFD4, YBL104C, |
| | YDR496C, YHR052W, YLR328W, YML076C, YNL294C, |
| | YPK2 |
| APM3 | APL5, APL6, PLO2, SWI1, TRF5 |
| APM4 | BI2, CAF4, YJR072C |
| ARC40 | ARC18, ARC19, ARC35, ARP2, ARP3, GCY1, NIP1, |
| | NOP4, PDR13, POB3, RAD30, YLR241W, YNL040W |
| ARE2 | PTC1 |
| ARF1 | ARF2, SHE10, YNL083W |
| ARL3 | YKL206C |
| ARP2 | ADE3, ADR1, ARC15, ARC18, ARC19, ARC35, ARC40, |
| | AR07, ARP3, ATP3, BNA1, BNI1, CDC47, CDC54, |
| | CIN1, DBF4, DED1, DUR1, 2, ECM17, FET3, GCD7, |
| | GEA2, GFA1, GLG2, GSY1, HUL4, IMH1, KAP104, |
| | KAP122, MET18, MSS18, NMD5, PDR13, PST2, PUF3, |
| | RPN8, RVB1, SEC23, SEC26, STE5, TOM70, TRP3, |
| | YGR016W, YJR029W, YKR065C, YMR018W, |
| | YMR278W, YNL313C |
| ASC1 | KRI1, LCP5, MSS116, PRP43, RFA1, SIK1, SIR3, SWI5, |
| | YDL060W, YGR145W, YOR056C, ZIP1 |
| BEM3 | DOP1, YIL055C, YTA7 |
| BFA1 | FET4, KEX2, STE23, YGL121C |
| BMH1 | ADR1, BNR1, BOI2, CSR2, CYK3, GSY2, KCS1, NTH1, |
| | REG1, SOK1, STU1, SVL3, YFR017C, YIL028W |
| BMH2 | CSR2 |
| BRE1 | YHR149C, YPL055C |
| BUB1 | KAR4 |
| BUB2 | ISM1 |
| BUD13 | CLU1, KIP3 |
| BUD20 | ADH2, COF1, CPH1, GPI15, HHF1, HMO1, HTB1, |
| | HTB2, HYP2, LSM2, MAM33, MDH1, MGM101, OYE2, |
| | TEF4, YBL004W, YDR036C, YFL006W, YHR052W, |
| | YIR003W, YLR004W, YPL013C, AFG2, FYV4, HHF1, |
| | HTA1, HTB1, HTB2, KRE32, LHP1, MAM33, NMD3, |
| | NOG1, NOP12, NOP13, PRP43, PUF6, PWP1, RSM24, |
| | RSM25, YBL044W, YDR038C, YDR101C, YER006W, |
| | YGL068W, YGR103W, YHR197W, YJL122W, YPL013C |
| BUD32 | AAC3, CAR2, CPR6, CRM1, DIA4, GRX3, GRX4, HEF3, |
| | IDP2, IMD2, IMD4, PHO81, POR1, RPN1, RPN5, RPN6, |
| | RPT1, RPT3, SEC18, SEC23, URA7, YDR279W, |
| | YHR033W, YJR072C, YKR038C, YML036W, YMR226C, |
| | YOR073W |
| CAC2 | RLF2, YDR453C, YLR080W |
| CAF20 | CDC33, GAL83, NAP1 |
| CAF4 | ATP3, CCT2, CCT3, CCT5, CCT6, DPM1, ENT2, OSH7, |
| | PRE2, SRP54, TCP1, YBL029W |
| CAR1 | HYP2, IPP1, MDH1 |
| CBF5 | CRN1, MSS18, PAN5, SIK1, SRP1, VMA6, YIL104C, |
| | YNL124W |
| CBK1 | ARP2, ECM10, GAL7, MOB2, PRB1, SEC28, SGT2, |
| | SIS1, SSD1, TAO3, UBP15, VMA6 |
| CCE1 | RNR3 |
| CCR4 | CDC36, CDC39, CYS4, POP2, RNQ1, RVB1, STI1, |
| | UBR1, YGR086C |
| CCT2 | ARC35, SEN2 |
| CDC10 | CDC11, CDC12, CDC3, IMD1, LPD1, SES1, SHS1, |
| | TFG1, YPL191C |
| CDC11 | CDC10, CDC12, CDC3, CLU1, PDI1, RPN1, TIF4631, |
| | TIF4632, TOP2, YHR033W |
| CDC12 | CDC11, CDC3, DOG1, DOG2, IMD4, MET6, MSK1, |
| | PYC1, RGD1, SEC53, STB1, THI3, VMA22, YGL245W, |
| | YKL056C |
| CDC13 | BAT1, CPH1, ECM10, PRE6, SIP2 |
| CDC14 | AUK1, ATP3, ATP5, ATP7, DPM1, FUR1, GLC7, HEF3, |
| | HMS1, MCR1, PDR15, SNF4, SPE3, TPS1, VAS1, |
| | YDR453C |
| CDC15 | AUT2, TFP1 |
| CDC20 | CCT2, CCT3, CCT5, MAD3, MDH1, MKK2, TCP1 |
| CDC23 | HYP2, SWM1 |
| CDC28 | CLU1, GSY1, MET10, RPN1, TCP1 |
| CDC3 | AAT2, ARG3, ARO4, CDC11, CDC12, HCH1, HYP2, |
| | HYR1, NMD2, NTA1, TPD3, URA4, YBL032W, |
| | YDR287W, YFR011C, YPL176C |
| CDC33 | EAP1, FAA4, FRS2, GSY2, MKT1, RTT101, SLY1, |
| | SNF4, TRP2, YDL239C, YDR214W |
| CDC4 | SKP1 |
| CDC42 | ADH2, BEM4, CIK1, KCC4, SAN1, YBL032W, |
| | YHL013C |
| CDC5 | IRR1, KAP95, MCD1, NOP13, SMC1, SMC3, SPC72, |
| | SRP1, YDR229W |
| CDC53 | PDC6, POL30, POR1, PTC1, SKP1, YBR280C, |
| | YLR352W |
| CDC55 | CCT2, CCT3, CCT5, CCT6, GPD1, GSY1, HFI1, MSN4, |
| | PDC6, PPE1, PPH21, PPH22, TCP1, TFG1, TPD3, YCK2, |
| | YER077C, YHR033W |
| CDC7 | BFR2, BIR1, ECM10, NUT1, PDC5, PDC6, PST2, |
| | RPC19, SAR1, SEC27, STI1, THI3, TPS1, UBI4, |
| | YLR231C, YLR331C, YLR386W |
| CDC9 | DBP9, ECM10, POL30, YOR378W |
| CDH1 | CCT2, CCT3, CDC28, CLB2, NA1, UBP15 |
| CHK1 | CTR1, GFA1, YLR152C |
| CIK1 | CLU1 |
| CKA1 | CKA2, CKB1, CKB2, DBP10, DBP2, EGD1, ERB1, |
| | HAS1, HHF1, HHT1, HOT1, HTA1, HTB2, KRE33, |
| | KRI1, MGM101, NOG1, NOP12, NOP2, NOP4, NPI46, |
| | PDI1, POB3, POL2, PUF6, PWP1, RRP5, SFP1, SIK1, |
| | SPT16, SSF1, TIF4631, TIF6, TRL1, WTM2, YOD116C, |
| | YER006W, YER084W, YGL104C, YGR090W, |
| | YGR103W, YGR145W, YHL035C, YHR052W, |
| | YKL082C, YLR002C, YPL110C, YRA1 |
| CKA2 | CKA1 |
| CKS1 | BUR2, CDC28, CLB2, CLB3, CLB5, CLN1, HYP2, |
| | YDR170W-A, YER138C |
| CLB2 | CDC28 |
| CLN1 | CDC2B, CKS1, PGM2 |
| CLN2 | ATP3, CDC28, ECM10 |
| CMD1 | CMK2, CMP2, COF1, CPH1, EDE1, HCH1, HUL5, |
| | HYP2, ILS1, IPP1, MLC1, MYO2, MYO3, MYO4, |
| | MYO5, NUF1, PGM2, PST2, SHE3, SHE4, SOD1, UBA1, |
| | VAS1, VPS13, YNK1 |
| CMK1 | CMD1, VPH2 |
| CMP2 | CMD1, CNB1, IDH1, RFC3, RPN7, TEF4 |
| CNA1 | CMD1, YGR263C |
| CNB1 | CMP2, CNA1, KRE6 |
| CNM67 | CAF4, FCP1 |
| CNS1 | ECM10, ILV5, YHB1 |
| COF1 | AIP1, CRN1, CYR1, GCN1, KAP114, PHO81, REX2, |
| | SRV2, TOS3 |
| COP1 | ADR1, ATP3, CET1, HFI1, OSH1, PRP6, RET2, RGA1, |
| | SEC21, SEC26, SEC27, SEC28, SPE3, TRP3, YBR270C, |
| | YER140W, YJR072C, YLR405W, YPL222W |
| COQ7 | COR1, IME4, PRP28, YJL068C |
| CPR6 | ADH2, CAF120, QNS1, TRR1, YOR154W, YOR220W |
| CSE2 | CDC33, POR1 |
| CTF13 | ARF1, SKP1 |
| CTK1 | CDC37, GBP2, HHF1, HRB1, KRE33, NPL3, SFP1, SIT4 |
| CTK3 | RVB1, STB3, UBA1, YBL032W |
| CYR1 | CDC33, RNR2, SRV2 |
| DBF2 | CYR1, FAA1, GPH1, MOB1, RPN5, RPT3, RPT5, |
| | SEC27, TFP1, TPS1, YJR072C |
| DBF20 | ALA1, AXL1, EGD2, GPH1, IDH2, MOB1, RPB10 |
| DBP8 | CAR2, CDC15, CPA2, HEF3, KGD1, OYE2, PFK1, |
| | PGM2, RNR1, RNR2, RPN1, SEC26, THI22, TIF2, |
| | YDL086W |
| DDC1 | MEC3, RFC4, SUV3 |
| DIA2 | BMS1, CDC46, CDC53, CKS1, COF1, CTF4, DBP10, |
| | DED81, ENP1, ILS1, KRE33, KRI1, LST4, MCM2, |
| | MCM3, NIP7, NMD3, NOP12, NPI46, PDR13, SEH1, |
| | SKP1, SLT2, SPB1, SSF1, SSF2, TIF6, YAK1, YBL104C, |
| | YHR052W, YJL109C, YKL014C, YPL012W |
| DIG2 | ACO1, KSS1, SRP1 |
| DMC1 | ACC1, DPM1, HNM1, MDJ1, MES1, POR1, TRP3, |
| | YDL148C, YDR516C, YLR106C |
| DPB11 | NMD3, SRP1, TIF4631 |
| DRC1 | ADH2, ADH4, CKA1, COP1, GAL7, IPP1, MAM33, |
| | MDH1, MGE1, MSU1, SRP1, TALl, TRL1, YHR074W |
| DSS4 | AFG2, FAA4, NOG1, SEC4, YDR101C, YGR103W, |
| | YJL122W, YPT1 |
| DUN1 | AAT2, ANC1, ASN2, DED81, PDX3, PRE8, VMA4, |
| | YDR214W, YFL030W, YGR086C |
| DUR1, 2 | BOI1 |
| ELA1 | EBP2, ECM10, ERB1, HAT1, IMD3, IMD4, KRE33, |
| | LOC1, MSS116, NOG1, NOP1, NOP12, PET127, PUF6, |
| | PWP1, TIS11, YAK1, YER077C, YGR086C, YGR090W, |
| | YGR103W, YHR052W, YKR081C, YPL004C, YPL012W, |
| | YRA1, YTM1 |
| ELM1 | TFP1 |
| ELP2 | ELP3, IK13, JIP1, ZMS1 |
| ERB1 | ACO1, CCT6, CDC14, EGD2, GND1, HAS1, HXT7, |
| | MET6, MRT4, MUB1, NOG1, PRP43, SAH1, SCS2, |
| | SEC53, SPB4, SSQ1, TIF6, UBR2, YER006W, |
| | YGL111W, YGL245W, YLR002C, YOR206W, YTM1, |
| | ARP2, BRX1, CRN1, EBP2, EXG1, FPR4, MRT4, MYO1, |
| | NMD3, NOG1, NOP2, PIB2, RLP7, SCS2, TIF6, |
| | YDR412W, YER002W, YGR103W, YHR052W, |
| | YKR081C, YLR002C, YNL110C |
| ESS1 | BCY1, CAR2, CPR6, HEF3, HSP104, HXT6, PUP2, |
| | RPB3, RPN1, RPO21, SPT5, TFG1, TOM1, YGR090W, |
| | YHR033W, YLR106C |
| EST1 | CBF5, DBP7, HSH49, KRE33, MSS116, PDI1, PET56, |
| | PUF6, PWP1, RRP1, YER077C, YJL109C, YKL014C, |
| | YKR081C, YPL012W |
| FAA4 | PSR2 |
| FAP1 | FPR1 |
| FAR1 | CLU1, COP1, RPT3, SRP1, SSK2, UBP15 |
| FHL1 | FKH1, FKH2, GCN3, HHF1, HMO1, HTA1 |
| FKH1 | CDC28, CEG1, CKA1, CKA2, CKB1, CKB2, FHL1, |
| | FKH2, FYV8, GCD2, GCD7, GCN3, HHF1, HTB1, |
| | MBP1, MGM101, MPH1, NET1, NOP1, RRP1, SEC2, |
| | SIN3, SUI2, SUI3, UBP12, URE2, YGR017W, YMR144W |
| FKH2 | ADH2, HTB2, INO80, SIN3 |
| FPR1 | AAT2, ADE3, ALA1, ASN2, CIN1, DED81, GDI1, |
| | HOM3, HSH49, KRS1, LIP5, MLP2, MSK1, PDR13, |
| | PET127, PRP28, THI3, THS1, URA1, YDR341C |
| FUM1 | YHR113W |
| FUN11 | CLU1, RPN1, TIF2 |
| FUN31 | GPH1, RVB1, YOL045W |
| GBP2 | HPR1, IMD3, MFT1, RLR1, SUB2, THP2, YNL253W, |
| | YRA1 |
| GCD11 | BNI1, CDC123, GCD1, SPT16, YDL172C, YNL091W |
| GCD2 | GCD1, GCD6, GCD7, GCN3, PRP6 |
| GCD7 | FAA4, FET3, GCD1, GCD11, GCD2, GCD6, GCN3, |
| | LOS1, MET18, MSH4, NMD5, PRO3, SAN1, SCW4, |
| | SUI2, SUI3, VAC8, YAF9, YLR243W |
| GCN2 | YNL213C |
| GCN3 | BGL2, CBP6, CDC39, CRN1, DHH1, ENP1, FET3, FRS2, |
| | GAL2, GCD1, GCD11, GCD2, GCD6, GCD7, GUF1, |
| | HIG1, IMH1, ITR1, KGD1, LCB1, MAS6, MCX1, |
| | MGM1, MKT1, NDI1, PET9, PRE2, PRE9, PRP16, |
| | RPB11, SAN1, SCY1, SDH2, SDH4, SEC34, SLC1, |
| | SPT15, TOM70, TRP2, TRP3, VPS8, YBR0140, |
| | YGL101W, YHM2, YJR072C, YJR080C, |
| | YKR046C, YKR065C, YOL101C, YPL207W |
| GCN5 | ADA2, FET4, HFI1, SPT7, TAF60, TRA1, UBP8, |
| | YCL010C |
| GDI1 | SEC4, STE11, VPS21, YPT1, YPT10, YPT31, YPT32, |
| | YPT52, YPT6, YPT7 |
| GIP2 | GDB1, GLC7, GPH1, GSY2, MDH1 |
| GLC7 | CFT1, CLU1, CYS3, ERB1, FIN1, FIP1, FPR4, GLC8, |
| | GPH1, GSY1, GSY2, KEL1, MDH1, MHP1, NPI46, |
| | PRC1, REG1, SCD5, SDS22, SEN1, SPB1, STI1, SUI2, |
| | SUI3, TRR1, YAR014C, YDR412W, YFR003C, |
| | YGL111W, YGR103W, YHR052W, YOR227W, YTM1 |
| GLC8 | GLC7, PHO85, PPZ2 |
| GND1 | HEF3, KIP3, MOH1 |
| GPA2 | GPA1, IDH1, PMA2, YGL245W, YMR029C |
| GRR1 | CDC53, COF1, CPH1, FOL2, HTB2, PDC5, PDC6, PFK1, |
| | POR1, SAH1, SKP1, UBI4 |
| GSP1 | DJP1, GSP2, HOM3, KAP95, MOG1, RHO1, RHA1, |
| | SNF12, SRM1, YDL172C, YRB1 |
| GYP6 | TRS120, TRS130 |
| HAL5 | ITR2 |
| HAP2 | AAC3, APG17, ARP4, ATP3, CDC33, CIS1, CYS4, |
| | FOL2, GRH1, HAP5, IPP1, KRE32, LOC1, MAM33, |
| | NAP1, NMD5, POL5, PSE1, RHR2, SAH1, SAP190, |
| | SPE3, SSK2, TIF2, TIF6, YER002W, YHR052W, |
| | YKL214C, YNL063W, YPL166W, YPR085C, YRA1, |
| | YTM1 |
| HAP3 | GSF2, SPT4, TFP1, YOR203W |
| HAT2 | ARC35, BAS1, DIM1, GND1, HAT1, HIF1, YOR233C, |
| | YPR105C |
| HEX3 | PTP2, SSK1, SSK2 |
| HIR1 | YER066C-A |
| HOG1 | RCK2, VID21 |
| HPR1 | MFT1, PRB1, RLR1, SSK2, SUB2, YDR214W |
| HPR5 | DUN1, SEC23, SEC53 |
| HRR25 | AEP1, ACO1, ATP4, BUD14, CAR2, CDC25, CKA2, |
| | COR1, CRZ1, CYS4, DCP1, DCP2, DNM1, EDE1, EGD2, |
| | ENP1, GAS1, GCN3, GLC7, GPH1, HHF1, HSP104, |
| | HXT7, HYP2, IPP1, LOC1, LTV1, MDH1, MDS3, MGE1, |
| | NPI46, OYE2, PEX19, PIN4, PTC4, PUF3, RPC19, |
| | RPM2, SAP185, SAP190, SAS10, SEC2, SEC23, SES1, |
| | SFB3, SGM1, SIT4, TGL1, TSR1, VMA4, YBR225W, |
| | YEL015W, YER006W, YER1380, YGL111W, YGR086C, |
| | YKL056C, YNL207W, YOR215C, YPL004C |
| HRT1 | ADR1, BBC1, CDC39, CDC53, CRM1, DUR1, 2, ECM29, |
| | ECM33, FAA4, GAL3, GCN1, GUF1, HYP2, IDH1, |
| | KIM3, MKT1, MYO2, PFK1, PMA2, RPA190, RPN1, |
| | RPN8, RTT101, SEC27, TPS1, UBI4, VPS13, YAR009C, |
| | YGP1, YLL034C, YLR035C-A, YLR106C |
| HSH49 | CHD1, CPH1, MLC2, RSE1 |
| HSP12 | CPH1 |
| HTA1 | HHF1, HIR2, HTB1, KAP114, KRI1, NAP1, NOP4, |
| | RET1, RPC82, RPO31, SPT16, YGR103W, YLR222C |
| HYM1 | ADH2, FET3, KIC1, PEX19, UFD4 |
| IME2 | CCT2, TCP1 |
| IMH1 | BI2, ERG13 |
| INO4 | HHF1, HTB1, HTB2, MAM33, NIP7, NUD1, PSE1, |
| | YDL001W, YDR324C, YGL099W |
| INP52 | ADO1, POR1, RNQ1, SAP190, TIF2, YDR279W, YHB1 |
| IST3 | BUD13, CAR2, CPH1, DED81, PDR13, PGM2, SAH1, |
| | YDR341C |
| ISU1 | NFS1 |
| ISW2 | ISW1, KAP95 |
| KAP104 | AAC1, ABF2, ADR1, ALD2, ALD3, ARF2, ARP2, |
| | AYR1, BGL2, CBP6, CKA2, COX2, DBP6, DED1, DIM1, |
| | DOG2, DPS1, EMP47, ENP1, ERP1, GAR1, GSF2, GSP1, |
| | GSP2, GTT1, HEM15, HFI1, HRP1, HTA2, ISA1, KEM1, |
| | KTR3, MAK16, MAS6, MEP1, MLC1, MNN9, MNT3, |
| | MRT4, NAB2, NDI1, NUP170, NUT1, QAC1, PAB1, |
| | PCL8, PET9, PGM2, PMD1, PSD1, RHO1, RMT2, |
| | RPC40, SAC1, SEC4, TFG2, TIM11, TOM20, TUS1, |
| | WSP1, YBR270C, YDL063C, YDL113C, YDL114W, |
| | YDL204W, YDR071C, YDR275W, YER182W, |
| | YFL027C, YHM2, YKR046C, YNL035C, |
| | YNR021W, YOR093C, YPL138C, YPR13SC, YRB1 |
| KJN2 | BUD14, CMP2, DOG1, GIS4, HSP104, KEL1, KEL2, |
| | KRE6, POP2, TEF4, TFC4, UBA1 |
| KIN28 | MPS2, SCJ1 |
| KIN82 | YNR047W |
| KNS1 | CAR2, TFP1, TPS1 |
| KRE31 | BRX1, BUD3, CCT2, CCT3, CCT6, CIN8, CLU1, HIR1, |
| | HTB1, KRE33, NAN1, POL5, RVB1, SIK1, SSD1, |
| | TIF4631, YGL068W, YGR090W, YJL109C, YKL056C, |
| | YPL012W |
| KSP1 | ARO1, BCK1, CHS1, CMP2, DBP7, PRI2, TPD3, |
| | YHR186C, YNL201C |
| KSS1 | ACO1, ARP7, BCK2, BEM3, CCT2, CYS4, DIG1, DIG2, |
| | FET4, FUS3, GFA1, HAS1, HXT6, MKT1, MSE1, NAP1, |
| | PHO84, PIM1, PMA1, PYC1, RPA135, RPN10, RPN8, |
| | RVB1, SEN1, STE11, STE12, STE7, TEC1, UBI4, |
| | YDR239C, YER093C, YGL245W, YHR033W, YJR072C, |
| | YLR154C, YOL078W, YPR115W |
| LAP4 | AMS1, BIK1, CPH1, DLD3, FUS2, GGA1, GLN1, HHT1, |
| | HTB1, MPP10, SPO72, SUP45, UBP15, VMA4, |
| | YDR131C, YFL034W, YNL045W, YOL082W |
| LAS17 | AAC3, BZZ1, GAL2, HXT6, HXT7, MYO4, PEP1, |
| | PHO84, RPN1, RPN12, RVS167, SLA1, SQT1, VMA6, |
| | VRP1, YHM2, YNR065C |
| LCD1 | ADH2, ADH5, HEM15, HHF1, ILV5, RNQ1 |
| LEM3 | GCN1, HXT7, KAP95, NMD5, TCP1, YHR199C, |
| | YLR326W |
| LIF1 | ANP1, CKA2, DNL4, MEC3 |
| LIG4 | ACO1, HTA1, KGD1, MAK16, NOP2, TIF6, YDR198C, |
| | YGL111W, YGL146C, YGR103W, YHR052W, |
| | YKR081C, YNL110C, YPL110C, YTM1 |
| LSM2 | ADE5, 7, DHH1, LSM1, LSM4, LSM7, LSMB, PAT1, |
| | PRP24, RPN6 |
| LSM4 | PAT1, SEC26, TPS1, UBP15 |
| LSM8 | APA1, GAR1, LSM2, QCR2, RPN12, RPN8, RRP42, |
| | SMB1, TIF6, YGL117W |
| LST8 | YFR039C |
| LTP1 | MOT1 |
| LYS1 | FOL2, POR1, TAL1 |
| MAG1 | AI1, FUN12, HHO1, HHT1, HTB2, IMD2, IMD3, IMD4, |
| | MPH1, MSH2, NOP12, RET1, RPC82, TIF4631, T1F4632, |
| | YGR090W |
| MAK11 | ERB1, HUL5, NOP2, TIF6, YGR103W |
| MCD1 | IRR1, SMC1, SMC3 |
| MCK1 | PNT1, TRM3, YIL105C |
| MDH2 | EXG1, FAA4, IMH1, MET18, PDI1, RFC2, RPN5, |
| | YIL108W, YMR093W |
| MEC1 | AC01, CLU1, MDH1, STI1, YGL245W |
| MEC3 | RAD17 |
| MED4 | NIT1, PEX6, ROM1, TOF1, YMR102C, ZRG17 |
| MEK1 | MSN2, NMD3, RPN1, TFG1, YMR323W |
| MET18 | BCY1, PRB1, RAD3 |
| MET30 | CDC53, HRT1, MET31, MET4, RUB1, SIS1, SKP1, |
| | TEF4, UBI4 |
| MGT1 | AHP1, ARF1, CPH1, DUN1, GND1, HEF3, HHT1, HTB1, |
| | LHS1, MGE1, RIP1, SEC27, SOF1, UBR1, YDR214W, |
| | YGL121C, YHR033W, YKL056C |
| MHP1 | GLC7 |
| MIG1 | MSS116, NOP12 |
| MIH1 | C0R1, CPH1, HTB2, QCR2 |
| MKK2 | ARP2, BCK1, BUL1, IDH1, LYS12, PRB1, RGD1, |
| | RNQ1, RPN1, RPN7, RVB1, YJR072C |
| MLH1 | MGE1, YOR155C |
| MLH3 | GCR2 |
| MMS2 | IRA2, RSP5, UBC13, YOR220W |
| MOB2 | CBK1 |
| MSG5 | FUS3, KSS1, SLT2, TAL1 |
| MSH1 | MAS1, MAS2 |
| MSH3 | HTB1 |
| MSH6 | MSH2 |
| MSI1 | CRC1, RLF2, YKR029C |
| MSN5 | GAL11 |
| MUS81 | ADH2, ANC1, CDC16, CDC33, CDC5, ERB1, HHF1, |
| | HHO1, KRE33, KRI1, LOC1, MES1, MGM101, MKT1, |
| | MMS4, NHP2, NOP12, NOP2, NP148, PWP1, RAD53, |
| | RPC10, SEC23, UBI4, YER006W, YER078C, YGR090W, |
| | YHB1, YKR081C, YMR226C, YRA1 |
| NAN1 | OND1, GND2, SAP1 |
| NMR1 | APG2, CDC55, FAT1, IFM1, SAP155, SIT4, SKT5, |
| | TIM22, XRS2, YDL121C, VDR287W |
| NOP13 | DBP7, DRS1, EBP2, IMD2, IMD3, KRI1, MSS116, |
| | NOP4, PUF6, RRPS, TIF4631, YGRS03W, YHR052W, |
| | YOR206W |
| NOP2 | BRX1, CKB1, COX6, FET4, GAR1, KRE32, NIP7, |
| | NMD3, NOP1, PHO84, RRP1, SIK1, YER006W, |
| | YGL111W, YGR103W, YOR206W, YPL009C |
| NPR1 | SIP2, UBP14 |
| NTA1 | ECM10, HSP104, IPP1, MDH1, MGE1, TFP1, VMA4 |
| NTG1 | ARP2, CLU1, ECM10, FET3, IDH1, PRB1, RFC2, |
| | RPC40, TIF34, YDR214W |
| NUP84 | NUP120, NUP145, NUP85, OPI3, SLU7 |
| NUP85 | CBP3, HEM15, NUP84, SEHI, YMR209C |
| OSH3 | NOP4 |
| PAC1 | PH06, YPK2 |
| PAC11 | DYN2, EXG1, PTC4, YBL064C, YLR177W, YOR172W |
| PAC2 | RPN1 |
| PAT1 | DCP2, DHH1, LSM1, LSM4, LSM7, PEX19, YGL121C |
| PBS2 | FET4, NEP2, PTC1, SSK2 |
| PCL6 | PHO85 |
| PCL9 | COR1 |
| PDS1 | SRP1 |
| PEP3 | PTC1, SEC7 |
| PEX7 | BNI1, CCT2, CCT3, CCT5, CCT6, CYS3, ENT4, FZO1, |
| | LAP4, LST8, MYO2, NEW1, PRI1, RPN6, SEC6, SEN2, |
| | SIF2, UBR1, YFL042C, YIL077C, YKL018W |
| PFK2 | PFK1, TIF2 |
| PFS2 | CCT2, CCT5, CCT8, CFTl, HGH1, TOP1 |
| PHO85 | AAC1, ADK1, CDC26, FZO1, GSP1, PCL10, PCL6, |
| | PCL7, PHO81, RHC18, SRP68, TOM20, VMA5, |
| | YDR214W, YDR453C, YER083C, YFL030W, YGR165W, |
| | VHB1, YML059C, YNL127W |
| PHR1 | MSD1 |
| PIB1 | UBI4 |
| PKH1 | TPK3, YGR088C, YPL004C |
| PKH2 | HXT6, HXT7, YGR033C, YGR086C, YPL004C |
| POL30 | CVS4, IMD4, MKK2, RPO31 |
| POL4 | RHO5 |
| PPH21 | HEM15, PPE1, PPH22, RPC40, RTS1, TAP42, TPD3, |
| | YGR161C |
| PPH22 | CDC55, HTA2, MKT1, PPE1, RPA135, RPB11, RTS1, |
| | RVB1, TAP42, TPD3, YGL121C, YGR161C |
| PPH3 | CCT2, CCT3, DIA4, STE12, TCP1, YBL046W, |
| | YHR033W, YNL201C |
| PPS1 | ADE13 |
| PPZ2 | GLC8, SDS22, YOR054C |
| PRE1 | PRE10, PRE2, PRE3, PRE5, PRE6, PRE7, PRE8, PRE9, |
| | PUP2, PUP3, SCL1, YHR033W, YKL206C, YLR199C |
| PRK1 | ABP1, AKL1, ECM10 |
| PRP11 | ADH2, ADK1, CLU1, COP1, GPH1, NAN1, REX2, |
| | SEC27, SHM2, SSK2, TEP1, THI22, TIF4631, UBP15, |
| | YGR043C, YGR250C, YLR222C |
| PRP19 | CEF1, CLF1, SNT309 |
| PRP4 | ARP2, CSE1, STI1, TOR1 |
| PRP46 | CCT5, CCT6, PFK1, SGT2 |
| PRP6 | AAT2, ADE13, ADE16, ADE3, ADE6, ALA1, APE2, |
| | ARA1, ASN2, BAT1, BRR2, CLU1, CMD1, COX4, |
| | CPR6, CYS3, DED81, DOT6, FRS1, GCY1, GLN1, |
| | GPD2, GPH1, GUF1, HSL7, HYP2, ILV3, IMD3, KRS1, |
| | LEU4, MAD1, MDH1, MDNH3, MES1, MMD1, MSH6, |
| | MSK1, PAB1, PAC2, PDR13, PMI40, PRC1, PRO3, |
| | PRP3, PRP31, PRP4, RRM3, RRP1, SAM4, SCC2, |
| | SCP160, SIS1, TIF34, TRL1, TRR1, YMR099C, |
| | YNL123W, YOR214C, YOR285W, YPL004C, ZTA1 |
| PSO2 | MGM101, YHR076W |
| PSR1 | PHM7, WHI2, YSA1 |
| PSR2 | BRN1, BUL1, EXG1, HXT6, HXT7, SSL2, YOR352W |
| PTC1 | TSL1 |
| PTC3 | COP1, ECM29, REP1, YDR071C, YGR205W, YOR086C |
| PTC4 | GIN4, YDR071C, YDR247W |
| PTC5 | PRS3, TIF6 |
| PTP3 | FET4, HHF1, RRP5 |
| PWP1 | BRX1, CCT2, CCT3, CCT5, CCT6, HEM15, TCP1, |
| | YOL027C |
| PWP2 | YDR449C, YGR210C, YLR222C |
| QRI8 | AHP1, SIP2, SSK1, SSK2, TPK2, UBI4 |
| RAD1 | CAR2, DUN1, FAR1, GPD1, GPD2, MSI1, MSS18, |
| | PDC8, PWP2, SEC6, SEN1, STE20, UBI4, YAL027W, |
| | YDR324C, YGR086C, YHR033W, YLR368W, |
| | YNL116W, YPL004C |
| RAD10 | ARC1, CPH1, FUM1, PRO1, RAD1, RNR2, SAH1, |
| | SOD2, TFP1, TIF2 |
| RAD14 | CCE1, CTF4, RAD1, RAD16, RAD4 |
| RAD16 | GND1, HHF1, HTB2, HTZ1, PDX1, RAD7, SHP1, |
| | YDR453C, YMR226C |
| RAD2 | PEX15 |
| RAD24 | CCT3, DUN1, RFC2, RFC3, RFC5, RPT3, TCP1, |
| | YDR214W, YJR072C, YLR413W |
| RAD25 | MKT1, ST11 |
| RAD26 | ACH1, ACO1, ADH4, BIO3, CDC33, ECM10, ERG20, |
| | GDI1, MAM33, MDH1, QCR2, RAD3, RHR2, SEC53, |
| | TEF4, TFP1, TIF2, YDR326C, YHR076W, YMR226C, |
| | YMR318C |
| RAD27 | POL30 |
| RAD28 | CCT2, CCT6, DUN1, TCP1 |
| RAD3 | AAC1, AAC3, ACO1, ATP3, CCL1, HOR2, HXT6, IDH2, |
| | KIN28, LSC1, MDH1, MET18, RHR2, RPN1, RPN8, |
| | RPT3, TFB3, TFP1, THI22, YBR184W |
| RAD30 | GPH1 |
| RAD50 | DUN1, GPH1, MAM33, MKT1, MRE11, REX2, RPT3, |
| | SEC27, SSK22, TFP1, VMA8, XRS2 |
| RAD51 | MLH1 |
| RAD52 | ALD5 |
| RAD53 | ASF1, CDC13, DUN1, EDE1, HTA2, IPP1, KAP95, |
| | MDH1, PTC2, SMC3, SRP1, SWI4, TBF1, YDR071C, |
| | YGR090W, YMR135C, YTA7 |
| RAD54 | MDH1, MGE1, YKL056C |
| RAD55 | PTC3, YHR033W |
| RAD59 | AAC3, ATP3, BEM2, ECM10, GCD11, HOM3, HOR2, |
| | ILV2, NTG1, OPY1, OYE2, PGM1, PGM2, PRB1, PTC3, |
| | RAD52, RHR2, RPB3, RPT3, SEC27, SEC53, TEF4, |
| | UBA1, VMA8, YDR214W, YER138C, YGR086C, YPT31 |
| RAD6 | MED4, RAD18, UBR2, YGL057C, YMR251W |
| RAD7 | ELC1, UBI4 |
| RAD9 | DUN1 |
| RAS2 | IRA1, RAS1, TSR1 |
| RCK1 | CBR1, FUS3, HOG1, IDH2, ROD1, RPN8, SNF1, SNF4, |
| | YPR038W |
| RCK2 | FET4, HOG1, VPS41 |
| RED1 | SEC7 |
| RFA1 | ACO1, ARP2, RPT2, RVB1, YER078C |
| RFA2 | AHP1, CDC10, GCD11, HIR3, HTB1, MGM101 |
| RFA3 | AAC3, AR01, CYS4, HEF3, HOR2, HXT7, PGM2, RHR2, |
| | YDR128W, YJR141W |
| RFC2 | ACH1, ADE5, 7, ATP3, BRR2, CPA2, HEF3, PGM2, |
| | RFC3, RFC4, ROM2, SRP1, VAC8 |
| RFC3 | MAP2, RFC4, RFC5, RNQ1, RPN11, RPT3, SHM2, |
| | YCL042W, YMR226C |
| RFC4 | ACO1, ADE5, 7, ADH2, EFO1, HSP104, RFC1, RFC2, |
| | RNQ1, RPT3, SAN1, YDR214W, YGL245W, YHR020W |
| RHC18 | HHF1, IMD1, IMD4, SRP1 |
| RHO1 | AAT2, ASN2, CLF1, DIA1, DLD3, FUM1, GIS1, GLY1, |
| | ILV3, PST2, WTM1, YBL064C, YFR044C |
| RHO2 | MER1, MKT1, POR1, RRP5, VPS21 |
| RHO4 | NMD3, PDR13, RPG1, URA1 |
| RHO5 | TRR1 |
| RIM11 | CDC25, CKI1, GCR2, GIN4, HOM3, IRA1, IRA2, MYO2, |
| | MYO4, NAP1, PMD1, PRS2, PRS3, PRS5, TPS1, TSL1, |
| | YDR170W-A, YER138C, YER160C, YJR027W, |
| | YJR028W |
| RIM15 | PHO13, PHO85 |
| RIS1 | APG7, NOP2, TIF6 |
| RLF2 | KAP95, SRP1 |
| RNA1 | CAR1, GSP1, GSP2, KGD1, YRB1 |
| RNR3 | ARP2, CYS4, HTB1, MAS1, MAS2, MKT1, RNQ1, |
| | RNR1, RPN12, RPN9, YNL134C |
| RPA190 | RPA135, RPA43, RPB5 |
| RPC19 | HHF1, RET1, RPA12, RPA135, RPA190, RPC40, |
| | YFR011C |
| RPC40 | ACC1, ACH1, ADE12, ADH2, ADK1, ADR1, ARF1, |
| | ARF2, ARO4, BGL2, CDC60, DOP1, ECM29, FRS1, |
| | GCD11, GFA1, GLT1, GLY1, GND1, GPD2, HTS1, |
| | IDH2, ILV1, ISA2, KAP122, KRI1, KRS1, MDH1, |
| | MET18, MGM1, NGL2, PAB1, POL30, PYC1, PYC2, |
| | RET1, RPA135, RPA190, RPA49, RPB5, RPC19, RPC25, |
| | RPC34, RPC82, RPN3, RPO26, RPO31, RVS167, SEC27, |
| | SMC4, SRY1, SSQ1, TBS1, TFP1, THS1, TOM40, URE2, |
| | VMA4, VMA5, XRS2, YDR214W, YDR453C, YER138C, |
| | YFL042C, YGL248W, YGR086C, YHR112C, YPL004C, |
| | ZUO1 |
| RPL5 | MLP1, RLP7, TIF6, YHR052W |
| RPN5 | EMP24, KGD1, KRE6, RPN1, RPN12, RPN6, RPN8, |
| | RPN9, RPT1, RPT2 |
| RPP0 | AHP1, HHF1, HXT7, HYP2, NMD3, TIF6, YER067W, |
| | YGL068W, YHR087W, YLR287C |
| RPT3 | ARP2, CPR6, HYP2, IDH2, LHS1, MKT1, NAS6, POR1, |
| | RPN1, RPN10, RPN11, RPN12, RPN3, RPN5, RPN6, |
| | RPN7, RPN8, RPN9, RPT1, RPT2, RPT4, RPT5, STI1, |
| | UBC12, YGL004C, YLR106C |
| RRP9 | CBF2, DYN1, JEM1, NET1, NOP13, NOP13, PRO1, |
| | YBL004W, YGL146C, YLR211C, YOL078W |
| RSP5 | BUL1, DUN1, HXT6, PHO84, RNQ1, RPB3, RPB5, |
| | RPO21, RPO26, YGR136W, YKR018C, YLR392C |
| RTF1 | SF17, YHR009C |
| RVB2 | RVB1 |
| RVS161 | ARG4, CRN1, DLD3, HSM3, MGE1, POR1, RVS167, |
| | YGL060W, YOR118W |
| RVS167 | COR1, DBP5, DBP9, DED1, ECM29, FRS1, FRS2, |
| | FUM1, GIP2, GPD1, HOM6, HYP2, IDH1, ILV5, KRS1, |
| | LPD1, LYS12, MAM33, MET18, NDI1, PDX3, PHO84, |
| | PMI40, PRE10, PRE9, RGA1, RNA1, RSP5, RVS161, |
| | SEC6, SER33, SES1, UBI4, UBP6, UBP7, URA7, |
| | YBL036C, YER138C, YHR022C, YLR243W, YPL249C, |
| | YSA1 |
| SAC6 | CNM67, LPD1, MDH1, SLF1, TRR1, XRS2, YER147C, |
| | YKL075C |
| SAL6 | SDS22 |
| SAN1 | ARP2, CDC54, RPA135, SRP1, UBI4, YPL113C |
| SAPI55 | FLR1, SAC1, SDF1, SIT4, TIM22, YDL113C, YLR222C |
| SAP185 | ANC1, ARG4, ARP4, ATE1, CDC33, CKA2, DUR1, 2, |
| | EPL1, ESA1, GSY1, HRR25, MPT1, PET9, POR1, PSD1, |
| | SDF1, YGR002C, YHM2, YMR209C, YPR040W, YRA1 |
| SAT4 | PHO85 |
| SDS22 | FYV14, GLC7, HXT6, NET1, NSR1, PMA1, PMA2, |
| | PPZ2, REG1, RSE1, RVB1, SNF4, YGR130C, YHR186C |
| SEC13 | NUP133, SEC31, YHL03PV |
| SEC27 | ARG4, ARG5, 6, AYR1, BIM1, BTN2, CCT2, CCT6, |
| | COP1, COR1, CPR6, CTR1, DNH1, EAP1, ERG27, |
| | FAA4, GAL7, GIC2, HFI1, IDH1, IML2, KAP122, MAE1, |
| | OM45, PCT1, PET9, PRB1, PRE10, PRO3, PTC3, RET2, |
| | RPN7, RPT3, RVS161, SEC18, SEC21, SEC26, SEC28, |
| | SEN54, STI1, TCP1, TIF34, TIF35, YBR187W, |
| | YCR076C, YDL204W, YER049W, YGR086C, YGR235C, |
| | YHR209W, YKR007W, YKR046C, YKR067W, |
| | YNL181W, YNR021W, YOR051C |
| SEC31 | CRN1, IDP3, SEC13 |
| SEH1 | ADE13, APE3, MYO1, NUP145, NUP84, NUP85, SEC13, |
| | SUB2 |
| SEN15 | AAT2, ACH1, ACO1, AFR1, AHP1, ARC1, ARF1, ATP3, |
| | CAR2, CDC33, CLU1, COF1, COR1, CPH1, CYR1, |
| | CYS4, EGD1, ERG13, ERG6, FPR1, FRS2, GND1, |
| | GND2, GRX1, HEF3, HHF1, LRO1, MET6, NTF2, OYE2, |
| | PFK1, PRM2, RNR2, RSN1, SAH1, SCP160, SEC53, |
| | SES1, SNU13, SOD1, TEF4, THS1, TIF2, UBA1, VMA4, |
| | VMA5, WTM1, VBR025C, YDR453C, YGL245W, |
| | YGR086C, YKL056C, YNK1, YPL004C |
| SET1 | BRE2 |
| SFP1 | LAS1, MRS6, RNQ1 |
| SGN1 | CLU1, FUN12, NPL3, PDI1, PUB1, SPT2, TIF4631, |
| | TIF4632, YGR250C |
| SHE2 | KTR3 |
| SHE3 | MLC1, MYO4, SUL2, SUP45 |
| SHS1 | ACC1, ARC35, ARP2, ATP3, BGL2, DIM1, GSY1, HIS4, |
| | MET3, MKT1, PUP2, RNQ1, RPB3, RVS167, SDH2, |
| | YHR033W |
| SIF2 | OSH2, TFP1, TRM3, VID28, YCR033W, YEL064C, |
| | YIL112W, YLR409C, YMR155W, YRF1-3, ZDS2 |
| SIP2 | ARC35, GAL83, IDH2, SEC53, SNF1, SNF4, TCP1 |
| SIR3 | COR1, CYS4, GAS1, ILV5, RNR2, SAH1, SES1, SIR1, |
| | TEF4, TFP1, TFP1, UBP8, YMR226C, YMR318C |
| SIR4 | BLM3, SEC53, SIR2, SIR3, SRP1, YFL006W |
| SIT4 | ACC1, ALG2, ARP2, ARP3, ATP3, BGL2, CCT6, |
| | CDC42, CDC47, CHL4, DED1, EXG1, FAA4, GAD1, |
| | GLT1, GSF2, HFI1, HXT3, HXT5, ILV1, MAE1, MSS18, |
| | PPH3, PRE1, PRE6, PRE9, RMT2, RPB3, SAP155, |
| | SAP185, SAP190, SCW4, TAP42, TIM22, WBP1, |
| | YDL204W, YDR380W, YGR161C, YHB1, YNR033W, |
| | YJR072C, YMR196W, YPR090W, ZRC1, ZWF1 |
| SIW14 | HXT6, YDR516C |
| SKI8 | AKL1, SKI2, SKI3 |
| SKM1 | HMG2, PTC1, TPD3 |
| SKP1 | BOP2, COC4, CDC53, PRB1, SGT1, UFO1, YDR131C |
| SKS1 | PRP28 |
| SLN1 | COP1, GCN3, LRS4, MDM1, VHR197W, ZRC1 |
| SLT2 | ARP2, BCK1, CPR6, EGD2, FOL2, GAL7, GND1, IDH1, |
| | ILV5, IPP1, KIC1, KIN2, LHS1, LYS12, MKK2, MKT1, |
| | OYE2, PDC6, PMA1, QCR2, RPN6, RPT3, SIS1, SMK1, |
| | TIF2, YDR214W, YGR086C, YLR187W, YOR220W |
| SMC1 | SMC3 |
| SMK1 | BUD7, COR1, GAL7, MAE1, PRE3, QCR2, RNR2, SLT2, |
| | STI1 |
| SML1 | AAC3, ADH3, ATP3, DUN1, ECM10, GPH1, HIR3, |
| | HOR2, NAT1, PFK1, PYC1, RNQ1 |
| SMT3 | CPH1 |
| SNF1 | ARF1, GAL83, GIS4, PRB1, SEC7, SIP2, SNF4, UBI4, |
| | YMR086W |
| SNF4 | GPH1, PST2, ROD1, SIP1, YOR287C |
| SNP1 | BCV1, COR1, DOG1, ENP1, FET4, HAS1, MAM33, |
| | NPI46, PIM1, PRP8, QCR2, SAP185, SAP190, SIT4, |
| | SRP1, YLR386W |
| SOF1 | CCT2, CCT3, CCT5, CCT6, KRE33, RRP5, TCP1 |
| SPC24 | BGL2, GCD11, GLT1, GPH1, ILV1, KAP122, MET18, |
| | NRG2, SPC25, TID3, TIM13, YER182W, YHR182W, |
| | YMR018W |
| SPC25 | CTF18, SPC24, YLR381W |
| SPO12 | PSE1, SRV2, SUM1 |
| SPO13 | IDH2, TIF2 |
| SPS1 | ARP2, ATP3, CPR6, IDH1, NMD5, PHO84, PPH21, |
| | PPH22, PRB1, REP1, RPN8, SDH2, VMA8, YDR214W, |
| | YDR372C, YHR033W, YKR046C |
| SPT2 | AAC3, CKA1, CKA2, CKB1, CYS4, GND1, GSP1, |
| | IMD2, KRE31, NOP1, NOP12, PUF6, RLI1, SAH1, SRP1, |
| | SSF1, STE23, SUP45, TIF4631, YGR090W, YKR081C |
| SPT8 | YML002W |
| SRP1 | BLM3, CNA1, CPR6, DIS3, EAF3, FIP1, FYV14, HAS1, |
| | HPR1, KAP95, MES1, MFT1, NAM8, NHX1, NUP1, |
| | NUP2, NUP60, PAP1, PCT1, PDS1, REB1, RLR1, RNT1, |
| | RRP4, RRP43, RRP6, RTT103, SIF2, SIN3, SNU56, |
| | STO1, TRA1, UME1, YPR090W |
| SSK1 | EST1, SSK2, SSK22 |
| SSK2 | DED81, DJP1, DPM1, GLT1, ILV1, LSC1, PTC3, |
| | TOM70, YCG1, YDL113C, YLR154C, YNL051W |
| STE4 | ADH2, ARP2, ASN2, CCT2, CCT3, CCT5, CCT6, |
| | GCD11, GPA1, LAP3, PDC6, RNQ1, SUI2, TCP1, THS1, |
| | VMA5, YDR214W, YHR033W |
| SUI1 | AAT2, ALA1, APE3, ARG4, ASN2, CDC60, COF1, |
| | DED81, ENP1, FUM1, HCH1, HYP2, MET14, NAS6, |
| | NIP1, PDC6, PDR13, PRP9, RPG1, RPO21, RPO31, |
| | SAR1, SAS4, SPT6, SUP45, TIF34, TIF35, TRR1, URA1, |
| | VID28, YGR169C, YKL056C, YOR177C, YPL067C |
| SUI2 | CDC33, FAL1, GCD1, GCD11, GCD2, GCD6, GCN3, |
| | RFA1, SPT16, SUI3, TIF2, TIF4631, T1F4632, VPS4, |
| | YBL032W, YLR400W |
| SWE1 | AHC1, CLB2, COP1, HSL7, KEL1, UBP15 |
| SWI5 | ARP4, FAA2, HFI1, RPO31, SPT7, STB4, TRA1, |
| | YGR002C |
| SWM1 | ARO1, ARP2, CPA1, PRB1, PRP28, RNQ1, URA7, |
| | YML072C |
| SXM1 | ECM1, LHP1 |
| TAF90 | CCT2, CCT3, CCT5, NTG2, RSC1, TCP1, YDR287W, |
| | YER160C, YJR072C, YNR065C |
| TEC1 | HHF1, HTB1, STE12 |
| TEL1 | YPL110C |
| TEM1 | ADK1, BFA1, CDC15, CDC33, CLU1, COF1, COR1, |
| | CPH1, CPR3, CYS4, DUT1, EFB1, FAA4, GCD11, |
| | HAS1, KGD1, LAP4, MCX1, NMD3, NUP53, PFK1, |
| | PST2, RNR1, RNR2, RVB1, SAR1, SEC53, SSD1, |
| | TEF4, TIF2, UFD4, VMA5, YER281C, YGL245W, |
| | YGR066C, YHB1, YHR033W, YMR226C, YNK1, YTM1 |
| TEP1 | ILV2, MLH3, MLP2 |
| TFB3 | RAD3 |
| TIF2 | CAC2, CDC33, MED4, MLP1, MSK1, NDJ1, RAD5, |
| | ROM2, TFG1, TIF4831, TlF4632, YJL107C |
| T1F34 | RPG1 |
| TOM1 | PDR15, PRP6 |
| TOP1 | ACO1, CLU1, RPC82, SPT16, YFR011C |
| TOP2 | CKA2, CKB1, DUN1, SIK1, YLR154C, YRA1 |
| TOS3 | SNF4, YKR096W |
| TPK1 | BCY1, FET4, RIM15, TCP1, TPK2, TPK3, VAC8, VPS13 |
| TPK2 | BCY1, ECM7, MST1, SEC28, TID3, TPK1, TPK3, |
| | YIL005W, YJR054W |
| TPK3 | ADE5, 7, BCY1, CPR6, GPH1, PFK1, SEC27, TPK1, |
| | YHR033W, YHR214W-A, YNL227C, YPT7 |
| TPT1 | KRE33, NOP1, YKR081C |
| TRF4 | IMD1, IMD3, IMD4, MTR4, NAP1, PSE1, SIK1, |
| | YDL175C, YIL079C, YPL146C |
| TUP1 | APG16, CDC42, CLU1, COS7, CYC8, ECM10, GPH1, |
| | NFS1, PET127, RLM1, SEC27, SPH1, SSY5, VID22, |
| | YHC1, YHR052W, YIL082W, YKL116C |
| UBA1 | FOL2, SER1, STI1 |
| UBC1 | ADK1 |
| UBC12 | ULAl |
| UBC13 | AR09, MMS2, RAD18, REX2, UBA1 |
| UBC4 | QCR7, UFD4 |
| UBC6 | ATP4, GCN1, LOS1, POL5, RVB1, SEC7, UBA1, |
| | YBL004W, YKL056C, YPT1 |
| UFD2 | DSK2, HMF1, NPL4, RAD23, SHP1, TSL1, UBI4, |
| | YDR049W |
| ULA1 | PPH22 |
| UME1 | MSH3, MSS116, RPD3, RRP5, SIN3, YBL004W, |
| | YKR020W, YOL114C, YPL158C, YPL181W, ZRT1 |
| URA3 | RNQ1 |
| VAN1 | CBR1, COX2, DPM1, HOC1, ISW2, KTR3, MNN9, |
| | NAP1, SCC3, SCJ1, SLC1, SPT15, WBP1, YJR072C, |
| | YLR243W, YTA12 |
| VPS21 | ARO3, CDC60, GDI1, GPX1, IMD2, MRS6, STE11, |
| | YML128C, YPT1, YPT52, YPT53 |
| VPS41 | PDR15, PEP3, PRP4 |
| VPS8 | MPS2, TFG1 |
| WHI2 | CSR2, HYP2 |
| WTM1 | CCT2, CCT3, CCT6, TOP1, WTM2 |
| WTM2 | CCT6, KAP104, MSS1, RNR2, RVB1, TSL1, WTM1, |
| | YJL069C, YOR283W |
| XRS2 | AHP1, CDC16, ERG20, MRE11, PST2, RAD50, |
| | YBR063C |
| YAK1 | AHP1, CDC39, DNM1, GDB1, RAD50, UBP15, UBR1, |
| | VPS1, YDR453C, YLR241W, YLR270W, YOR173W, |
| | YPL247C |
| YAR003W | RNR2, UB14 |
| YBL036C | ADK1, FET4, HXT6, HXT7, PDC6, SES1, YOL078W |
| YBL049W | CCT2, CCT3, FYV10, VID26, VID30, YCL039W, |
| | YDR255C, YMR135C |
| YER094W | CKA2 |
| YBR175W | HPR1, RPN1, SET1, SFP1, SGS1, SUV3, YOL045W |
| YBR203W | NAP1, SKP1 |
| YBR223C | RNQ1, SHP1 |
| YBR267W | YJL122W |
| YBR280C | AAH1, CDC53, PRB1, YBR139W |
| YCK1 | AAC3, ADH2, AHP1, APC1, BCY1, CAR2, CDC4, |
| | CYS4, FOL2, GND1, HYP2, ILV5, LYS1, MPC54, OYE2, |
| | OYE3, POR1, PPH21, PPH22, PST2, PYC2, RGR1, |
| | RLR1, SAH1, SIP2, SNO2, SOD1, SSN8, THI22, TIF2, |
| | TPD3, TPK2, TPK3, UBA1, VPS21, YBL108W, |
| | YBR028C, YCK2, YCK3, YGR111W, YGR154C, |
| | YHR112C, YJL207C, YMR226C, YPT53 |
| YCK2 | YCK1 |
| YCL039W | BUD5, CTF19, FUN14, FYV10, HXT7, MNN1, PXA1, |
| | SES1, SIF2, TFP1, UME1, VID24, VID28, VID30, |
| | YBLO32W, YBL049W, YDR255C, YIL097W, |
| | YIR020W-B, YMR135C, YOL087C, YPL1330 |
| YCR001W | RAD23 |
| YCR079W | CDC60, FAA1, HEF3, KGD1, MDH1, PRO2, PYC1, |
| | RAD1, TIF2, TPS1, VID31, YPL110C |
| YDL025C | CDC33, YAL049C, YGR016W, YHR009C |
| YDL060W | HTB1, NOP12, YER006W, YOR056C |
| YDL100C | DLD1, GSF2, LAP4, MNN1, MSN4, POR1, YBR014C, |
| | YER083C, YGL020C, YGRO86C, YLR154C |
| YDL156W | CCT2, CCT3 |
| YDL175C | ADH2, DED1, NPL3, QCR2, SES1, SRP1, YGR165W |
| YDL193W | GCN1, GSP1, GSP2, NMD5, PMA1 |
| YDL213C | CBF5, CBP2, CDC33, DBP7, DRS1, ERB1, GBP2, HAS1, |
| | HMO1, HTB1, HTB2, IMD1, IMD3, ISA1, KAP95, |
| | KRE33, KRI1, KRR1, MGM101, MSS116, NOP12, NOP2, |
| | NOP58, NPL3, PET127, POL5, PRP43, PUF6, PWP1, |
| | RLI1, RRP5, TIF2, TIF4631, TIF6, TRA1, TSR1, |
| | YBL004W, YER006W, YGL068W, YGR103W, |
| | YGR145W, YGR150C, YGR198W, YHR052W, |
| | YJL109C, YJR041C, YKL014C, YKR081C, |
| | YOR206W, YPL012W, YRA1, YTM1 |
| YDR128W | CCT6, CPR6, DIM1, FAR1, GSF2, GSY1, GUF1, MDJ1, |
| | NGG1, NPR2, POX1, RMT2, RNQ1, SEC6, SEH1, |
| | VMA6, YDL113C, YDR2330, YER182W, YHR033W, |
| | YJR072C, YNR018W, YPL207W |
| YDR131C | SKP1, YRB2 |
| YDR165W | CDC53 |
| YDR200C | FET4, PHO84, YFR008W, YGR066C, YMR029C, |
| | YPL004C |
| YDR219C | SKP1, YHR122W |
| YDR247W | MER1, NUM1, PTC4, SEF1, SKT5, SPT16, SYF1, TPS1, |
| | YDR071C |
| Y0R266C | CLU1, MGE1 |
| YDR267C | ANC1, DOG1, MET18, RPN8, UBP9, YBR030W, |
| | YLR349W, YLR392C, YOL111C, YOR164C, YPL068C |
| YDR306C | CDC53, MDH1, PGM2, SAH1, SKP1, SRV2, STI1 |
| YDR316W | BUD9, DAK2, THI22, VMA6, YBL104C |
| YDR339C | COR1, PMC1 |
| YDR365C | CDC33, CKA1, CKA2, CKB1, HTB1, IMD3, LHP1, |
| | MSS116, NOP12, PMA1, YCR087W, YDR102C, |
| | YJL207C, YKR081C, YNR054C, YRA1 |
| YDR398W | ACC1, CPR6, CES1, ECM8, FAA4, GUF1, RMT2, |
| | SEC28, SEC6, SGD1, YER138C, YGR210C |
| YDR482C | TPD3 |
| YER041W | FPR4, POL30, YKR081C |
| YER066C-A | NMD2, PEX19, STI1, TOM70, YBL049W |
| YER117W | BCP1, FET4, IMD4, YPL208W |
| YFL034W | YPL110C |
| YFR003C | GLC7, MGE1 |
| YFR016C | CAP1, CAP2, COF1, KOG1 |
| YFR024C-A | ARO1, CKB2, PRP12, UBP15, YFR024C, YJL045W, |
| | YLR422W, YOR042W |
| YGL004C | HSM3, NAS6, RPC40, RPN1, RPN10, RPN11, RPN13, |
| | RPN3, RPN5, RPN6, RPN7, RPN8, RPN9, RPT1, RPT2, |
| | RPT3, RPT4, RPT5, YKL195W |
| YGL081W | COP1, CYS4, GFA1, GSY1, NIP1, RFC4, SMC3, UBR1, |
| | URA7, YER006W |
| YGL131C | YLR413W |
| YGL220W | GRX3, GRX4, YLL029W |
| YGR052W | APC2, ARF2, HIS4 |
| YGR054W | HTB2, KRE33, NPL3 |
| YGR067C | CLU1, HTB2, MKT1, SCP160 |
| YGR103W | CKA2, CKB2, DBP10, HAS1, MAM33, NOP1, RRP1, |
| | RRP5, SPB1, SRP1, TIF6, YER006W, YKR081C, |
| | YPR143W, YTM1 |
| YGR173W | MOH1, YDR152W |
| YGR223C | ERG10 |
| YGR280C | PRP12, YPL110C |
| YHL010C | NHA1, YBL049W, YKR017C |
| YHR052W | EBP2, ERB1, KRE33, MAK5, MSS116, NOP2, NOP56, |
| | RRP5, YOR206W |
| YHR105W | VPS13 |
| YHR115C | YNL116W, YNL311C |
| YHR186C | VPH2 |
| YHR188C | ARF1, ARF2 |
| YHR196W | GND1, GPH1, HSP104, KGD1, NAN1, PFK1, SCS2, |
| | TPS2, YJL109C |
| YHR197W | ATP3, BUD3, HTB2, RPC19, YDR131C, YNL182C |
| YHR199C | AEP1, IFM1, PSE1, TRX2 |
| YIL007C | AC01 |
| YIL079C | DED1, HRB1, IMD4, NPL3, TRF4 |
| YIL113W | SLT2, SRV2 |
| YJL020C | CPH1, GSY2, HTB2 |
| YJL068C | TAL1 |
| YJL069C | CKA1, CKA2, CKB1, CKB2, DIP2, KRE33, LAS1, LCP5, |
| | NAN1, NGG1, NOP1, PRP40, PTC5, PWP2, RRP5, SIK1, |
| | TFP1, YDR449C, YGR090W, YJL109C, YML093C, |
| | YKR060W, YKR096W, YLR222C, YLR409C, |
| | YML093W, YOR1450 |
| YJL149W | CDC53, SKP1 |
| YJR061W | KKQ8 |
| YJR110W | RGR1 |
| YJU2 | CCT5, COR1, DED81, DUN1, EGD1, GCD11, NAP1, |
| | NMD3, PRP19, QCR2, SOD1, TCP1, TIF2, YNK1 |
| YKL018W | CRN1, TPS3 |
| YKL078W | NOP1, SEC27 |
| YKL161C | GFA1, SAH1 |
| YKL215C | HSP104 |
| YKU70 | ATP4, FRS2, HYP2, PEX19, RPT3, RVB1 |
| YKU80 | ACO1, ADR1, APT1, ARO1, ATP3, CCT3, CCT5, CLU1, |
| | COP1, CPA2, DHH1, DPB2, ECM10, FOL2, FUN12, |
| | GAL7, GPH1, IDH1, ILV2, LSC1, LST8, LYS12, MET16, |
| | MKK2, MSU1, OYE2, PDX1, PHO85, PHO86, POR1, |
| | PRE1, PST2, PUF3, PUP3, RPN12, RRP3, SIP1, SIS1, |
| | SLC1, SLX1, SOD2, SRP54, STI1, TEM1, TFC7, TPS1, |
| | VID31, VMA8, YBT1, YDR128W, YDR453C, YER077C, |
| | YGR266W, YHR033W, YJR072C, YKR051W, |
| | YLR271W, YML020W, YMR226C, YNR053C, |
| | YOL078W, YPR003C |
| YLR016C | BUD13, ILS1, SMC4, SRP1 |
| YLR074C | ADH2, COF1, CPH1, GPI15, HHF1, HMO1, HTB1, |
| | HTB2, HYP2, LSM2, MAM33, MDH1, MGM101, OYE2, |
| | TEF4, YBL004W, YDR036C, YFL006W, YHR052W, |
| | YIR003W, YLR009W, YPL013C, AFG2, FYV4, HHF1, |
| | HTA1, HTB1, HTB2, KRE32, LHP1, MAM33, NMD3, |
| | NOG1, NOP12, NOP13, PRP43, PUF6, PWP1, RSM24, |
| | RSM25, YBL044W, VDR036C, YDR101C, YER006W, |
| | YGL068W, YGR103W, VHR197W, YJL122W, YPL013C |
| YLR097C | ADH2, CDC53, GUF1, IDH1, SKP1, UBI4 |
| YLR186W | CAR2, OYE2, PHO81, YER030W, YPL004C |
| YLR222C | ARP10, DIP2, DIP5, FET4, MUM2, PGM2, POR1, SRV2, |
| | TFP1, YHR020W, YJL069C |
| YLR238W | FET4, PHO84, VMR029C |
| YLR247C | CIN5, EXO70, HHF1, HTA1 |
| YLR320W | ESC4, GDH2, RTT101 |
| YLR352W | CDC53, SKP1 |
| YLR427W | ARE1, CDC33, FET4, FUN12, GRS1, HAS1, IMD2, |
| | IMD3, IMD4, KRE33, KRI1, MGM101, MSC3, NOP12, |
| | NOP4, NPL3, OYE2, PDC6, TIF4631, TIF4632, |
| | YGR090W, YHR199C, YKR081C, YOR206W, YPL012W |
| YML029W | PEX6, PIM1, YLR106C |
| YML088W | CDC53, SKP1 |
| YMR049C | ACO1, CCT6, CDC14, EGD2, GND1, HAS1, HXT7, |
| | MET6, MRT4, MUB1, NOG1, PRP43, SAH1, SCS2, |
| | SEC53, SPB4, SSQ1, TIF6, UBR2, YER008W, |
| | YGL111W, YGL245W, YLR002C, YOR206W, YTM1, |
| | ARP2, BRX1, CRN1, EBP2, EXG1, FPR4, MRT4, |
| | MYO1, NMD3, NOG1, NOP2, PIB2, RLP7, SCS2, |
| | TIF6, YDR412W, YER002W, YGR103W, YHR052W, |
| | YKR081C, YLR002C, YNL110C |
| YMR093W | ERB1, ROK1, YBR281C, YHR052W, YJL109C |
| YMR291W | FUM1, VPS33 |
| YNL035C | KIN1 |
| YNL056W | YNL099C |
| YNL094W | ABP1, COF1, CPH1, FYV8, MDH1, OYE2, PGM2, |
| | YPL004C |
| YWL099C | SW14 |
| YNL116W | PHO84, STH1, YNL311C, YPL110C |
| YNL157W | CPH1, HTB2, SAH1 |
| YNL182C | APG17, HHF1, Q0032 |
| YNL260C | POR1, YNL008C |
| YNL311C | ERG1, RPT2, RPT4, RVB1, SKP1, STI1, UBI4, |
| | YHR115C, YNL116W |
| YOL045W | FUN30, FUN31 |
| YOL054W | EDE1, GAC1, HHF1, HTA1, HTA2, HTB1, HTB2, KNS1, |
| | MAM33, POB3, SPT16, YCR030C, YOR056C |
| YOL087C | ATP3, BEM2, COP1, EDE1, FOL2, HTB2, LHS1, NIP1, |
| | POR1, RPG1, SES1, SRV2, TIF34, TIF35, UBI4 |
| YOL128C | GSP2, MDH1, OYE2, TRR1 |
| YOR026W | GSY2, Q0092 |
| YOR227W | GLC7 |
| YOR353C | KIC1 |
| YPK2 | CDC33, PET112, PRB1, SNF1, TFP1, YEL023C, |
| | YGR016W |
| YPL150W | ARO4, CAR2, NAP1, OYE2, YGR086C, YPL004C |
| YPL170W | GUF1, PMA1 |
| YPL236C | UFD2 |
| YPR015C | CLU1, MAM33, PGM2, RET1, SXM1, YHR046C |
| YPR093C | FPR1, RPB11, RPB3, RPB9, RPO21, YOR131C |
| YPT1 | DSS4, GDI1, MRS8, SEC4 |
| YPT10 | GDI1, MRS6 |
| YPT33 | BCY1, CDC33, MRS6, POR1, TPK1, TPK3, VPS21, |
| | YNL227C, YPT52 |
| YPT6 | ACO1, GDI1, RGP1, RIC1, RNA1 |
| YRB2 | ORM1, DIA4, PRSS |
| YTA6 | TOP2, YGR086C, YPL004C |
| YTMI | ERB1, RPF1, SRP54, VPS35, YBR242W, YHR052W, |
| | YIL1370, YPD1 |

Bold protein names indicate those for which an interaction with the bait was confirmed in the literature using PreBIND.

TABLE 3
Comparison of HMS-PCI and HTP-Y2H datasets

| Datasheet | Interactions found in literature |
|---|---|
| HTP-MS/MS Spoke | 166 |
| HTP-MS/MS Matrix | 230 |
| Ito et al.⁴⁶ | 47 |
| Uetz et al.⁷ | 51 |

TABLE 4A
Proteins removed by filtering criteria (Protocol A)

| ORF Name | Gene | Description |
|---|---|---|
| YLR044C | PDC1 | pyruvate decarboxylase |
| YIL107C | PFK26 | 6-Phosphofructose-2-kinase |
| YAL005C | SSA1 | Heat shock protein of HSP70 family, |
| | | cytoplasmic |
| YLR259C | HSP60 | mitochondrial chaperonin, homolog of E. |
| | | coli groEL protein |
| YJR045C | SSC1 | Mitochondrial matrix protein involved in |
| | | protein import\; subunit of SceI endonuclease |
| YOL145C | CTR9 | involved in mitosis and chromosome |
| | | segregation |
| YDR499W | LCD1 | |
| YMR116C | ASC1 | G-beta like protein |
| YJR121W | ATP2 | F(1)F(0)-ATPase complex beta subunit, |
| | | mitochondrial |
| YOL086C | ADH1 | Alcohol dehydrogenase |
| YLL024C | SSA2 | member of 70 kDa heat shock protein family |
| YBR196C | PGI1 | Glucose-6-phosphate isomerase |
| YBL099W | ATP1 | mitochondrial F1F0-ATPase alpha subunit |
| YBR118W | TEF2 | translational elongation factor EF-1 alpha |
| YOL055C | TH120 | THI for thiamine metabolism. Transcribed in |
| | | the presence of low level of thiamine (10- |
| | | 8M) and turned off in the presence of high |
| | | level (10-6M) of thiamine. Under the |
| | | positive control of TH12 and TH13. |
| YNL064C | YDJ1 | yeast dnaJ homolog (nuclear envelope |
| | | protein)\; heat shock protein |
| YHR111W | YHR111W | moeB, thiF, UBA1 |
| YGL244W | RTF1 | Nuclear protein |
| YPL106C | SSE1 | HSP70 family member, highly homologous |
| | | to Ssa1p and Sse2p |
| YHR174W | ENO2 | enolase |
| YCR012W | PGK1 | 3-phosphoglycerate kinase |
| YFR053C | HXK1 | Hexokinase I (PI) (also called Hexokinase A) |
| YKL152C | GPM1 | Phosphoglycerate mutase |
| YCL018W | LEU2 | beta-IPM (isopropylmalate) dehydrogenase |
| YBR072W | HSP26 | heat shock protein 26 |
| YFL039C | ACT1 | Actin |
| YBR127C | VMA2 | vacuolar ATPase V1 domain subunit B |
| | | (60 kDa) |
| YLR180W | SAM1 | S-adenosylmethionine synthetase |
| YBR020W | GAL1 | galactokinase |
| YGR192C | TDH3 | Glyceraldehyde-3-phosphate |
| | | dehydrogenase 3 |
| YBR136W | MEC1 | similar to phosphatidylinositol(PI)3-kinases |
| | | required for DNA damage induced |
| | | checkpoint responses in G1, S\/M, intra S, |
| | | and G2\/M in mitosis |
| YFL037W | TUB2 | beta-tubulin |
| YJL008C | CCT8 | Component of Chaperonin Containing |
| | | T-complex subunit eight |
| YGL009C | LEU1 | isopropylmalate isomerase |
| YDR050C | TPI1 | triosephosphate isomerase |
| YDL126C | CDC48 | microsomal ATPase |
| YLR150W | STM1 | gene product has affinity for quadruplex |
| | | nucleic acids |
| YAL038W | CDC19 | Pyruvate kinase |
| YML085C | TUB1 | alpha-tubulin |
| YJL148W | RPA34 | unshared RNA polymerase I subunit |
| YBR221C | PDB1 | beta subunit of pyruvate dehydrogenase |
| | | (E1 beta) |
| YJL088W | ARG3 | Ornithine carbamoyltransferase |
| YMR186W | HSC82 | constitutively expressed heat shock protein |
| YBR035C | PDX3 | pyridoxine (pyridoxiamine) phosphate |
| | | oxidase |
| YLR418C | CDC73 | RNA polymerase II accessory protein |
| YJL130C | URA2 | carbamoyl-phophate synthetase, aspartate |
| | | transcarbamylase, and glutamine |
| | | amidotransferase |
| YER177W | BMH1 | Homolog of mammalian 14-3-3 proteins |
| YMR205C | PFK2 | phosphofructokinase beta subunit |
| YCL040W | GLK1 | Glucokinase |
| YDL055C | PSA1 | mannose-1-phosphate guanyltransferase, |
| | | GDP-mannose pyrophosphorylase |
| YLR340W | RPP0 | 60S ribosomal protein P0 (A0) (L10E) |
| YKL060C | FBA1 | aldolase |
| YGR254W | ENO1 | enolase I |
| YJR123W | RPS5 | Ribosomal protein S5 (S2) (rp14) (YS8) |
| YBR279W | PAF1 | RNA polymerase II-associated protein |
| YDL229W | SSB1 | cytoplasmic member of the HSP70 family |
| YER165W | PAB1 | Poly(A) binding protein, cytoplasmic and |
| | | nuclear |
| YNL178W | RPS3 | Ribosomal protein S3 (rp13) (YS3) |
| YBR181C | RPS6B | 40S ribosomal gene product S6B (S10B) |
| | | (rp9) (YS4) |
| YGL206C | CHC1 | presumed vesicle coat protein |
| YPL061W | ALD6 | Cytosolic Aldehyde Dehydrogenase |
| YGL173C | KEM1 | cytoplsamic 5′-to-3′ exonuclease. |
| YFL018c | LPD1 | dihydrolipoamide dehydrogenase precursor |
| | | (mature protein is the E3 component of |
| | | alpha-ketoacid dehydrogenase complexes) |
| YNL071W | LAT1 | Dihydrolipoamide acetyltransferase |
| | | component (E2) of pyruvate dehydrogenase |
| | | complex |
| YPL235W | RVB2 | RUVB-like protein |
| YGL253W | HXK2 | Hexokinase II (PII) (also called |
| | | Hexokinase B) |
| YPL258C | TH121 | THI for thiamine metabolism. Transcribed in |
| | | the presence of low level of thiamine (10- |
| | | 8M) and turned off in the presence of high |
| | | level (10-6M) of thiamine. Under the |
| | | positive control of THI2 and THI3. |
| YPL240C | HSP82 | 82 kDa heat shock protein\; homolog of |
| | | mammalian Hsp90 |
| YOR063W | RPL3 | Ribosomal protein L3 (rp1) (YL1) |
| YPL131W | RPL5 | Ribosomal protein L5 (L1a) (YL3) |
| YJR009C | TDH2 | glyceraldehyde 3-phosphate dehydrogenase |
| YHR082C | KSP1 | Ser\/Thr protein kinase |
| YNL209W | SSB2 | Heat shock protein of HSP70 family, |
| | | homolog of SSB1 |
| YMR076C | PDS5 | (putative) involved in sister chromosome |
| | | cohesion during mitosis |
| YBR031W | RPL4A | Ribosomal protein L4A (L2A) (rp2) (YL2) |
| YJL034W | KAR2 | Homologue of mammalian BiP (GPR78) |
| | | protein\; member of the HSP70 gene family |
| YDR385W | EFT2 | translation elongation factor 2 (EF-2) |
| YDR171W | HSP42 | heat shock protein similar to HSP26, |
| | | involved in cytoskeleton assembly |
| YJR077C | MIR1 | |
| YHR203C | RPS4B | Ribosomal protein S4B (YS6) (rp5) (S7B) |
| YFR031C-A | RPL2A | Ribosomal protein L2A (L5A) (rp8) (YL6) |
| YJL066C | YJL066C | |
| YLL045C | RPL8B | Ribosomal protein L8B (L4B) (rp6) (YL5) |
| YHL034C | SBP1 | Single-strand nucleic acid binding protein |
| YDR099W | BMH2 | member of conserved eukaryotic 14-3-3 gene |
| | | family |
| YML028W | TSA1 | thioredoxin-peroxidase (TPx)\; reduces |
| | | H2O2 and alkyl hydroperoxides with the use |
| | | of hydrogens provided by thioredoxin, |
| | | thioredoxin reductase, and NADPH |
| YBL072C | RPS8A | Ribosomal protein S8A (S14A) (rp19) (YS9) |
| YLR249W | YEF3 | EF-3 (translational elongation factor 3) |
| YDR502C | SAM2 | S-adenosylmethionine synthetase |
| YMR214W | SCJ1 | dnaJ homolog |
| YER110C | KAP123 | Karyopherin beta 4 |
| YOR151C | RPB2 | second largest subunit of RNA polymerase II |
| YGL048C | RPT6 | ATPase |
| YJL052W | TDH1 | Glyceraldehyde-3-phosphate |
| | | dehydrogenase I |
| YKL180W | RPL17A | Ribosomal protein L17A (L20A) (YL17) |
| YML124C | TUB3 | alpha-tubulin |
| YGL076C | RPL7A | Ribosomal protein L7A (L6A) (rp11) (YL8) |
| YFL016C | MDJ1 | DnaJ homolog involved in mitochondrial |
| | | biogenesis and protein folding |
| YCL064C | CHA1 | catabolic serine (threonine) dehydratase |
| YMR066W | SOV1 | (putative) involved in respiration |
| YDR148C | KGD2 | dihydrolipoyl transsuccinylase component of |
| | | alpha-ketoglutarate dehydrogenase complex |
| | | in mitochondria |
| YKL035W | UGP1 | Uridinephosphoglucose pyrophosphorylase |
| YOR374W | ALD4 | mitochondrial aldehyde dehydrogenase |
| YKL182W | FAS1 | pentafunctional enzyme consisting of the |
| | | following domains: acetyl transferase, enoyl |
| | | reductase, dehydratase and malonyl\/palmityl |
| | | transferase |
| YCL037C | SRO9 | RNA binding protein with La motif |
| YBL030C | PET9 | mitochondrial ADP\/ATP translocator |
| YHL033C | RPL8A | Ribosomal protein L8A (rp6) (YL5) (L4A) |
| YIL075C | RPN2 | RPN2p is a component of the 26S proteosome |
| YGL123W | RPS2 | Ribosomal protein S2 (S4) (rp12) (YS5) |
| YBR019C | GAL10 | UDP-glucose 4-epimerase |
| YJL177W | RPL17B | Ribosomal protein L17B (L20B) (YL17) |
| YPL231W | FAS2 | alpha subunit of fatty acid synthase |
| YGR282C | BGL2 | Cell wall endo-beta-1,3-glucanase |
| YER178W | PDA1 | alpha subunit of pyruvate dehydrogenase |
| | | (E1 alpha) |
| YNR001C | CIT1 | citrate synthase. Nuclear encoded |
| | | mitochondrial protein. |
| YJL111W | CCT7 | Component of Chaperonin Containing |
| | | T-complex subunit seven |
| YDL143W | CCT4 | component of chaperonin complex |
| YGL135W | RPL1B | Ribosomal protein L1B |

TABLE 4B
Proteins removed by filtering criteria (Protocol B).

| ORF | Gene Name | Description |
|---|---|---|
| YGL009C | LEU1 | isopropylmalate isomerase |
| YAL005C | SSA1 | Heat shock protein of HSP70 family, |
| | | cytoplasmic |
| YOL055C | TH120 | THI for thiamine metabolism. Transcribed in |
| | | the presence of low level of thiamine (10- |
| | | 8M) and turned off in the presence of high |
| | | level (10-6M) of thiamine. Under the |
| | | positive control of THI2 and THI3. |
| YCL018W | LEU2 | beta-IPM (isopropylmalate) dehydrogenase |
| YLL024C | SSA2 | member of 70 kDa heat shock protein family |
| YAL038W | CDC19 | Pyruvate kinase |
| YLR044C | PDC1 | pyruvate decarboxylase |
| YHR174W | ENO2 | enolase |
| YGR192C | TDH3 | Glyceraldehyde-3-phosphate dehydrogenase 3 |
| YGR254W | ENO1 | enolase I |
| YBR118W | TEF2 | translational elongation factor EF-1 alpha |
| YOL086C | ADH1 | Alcohol dehydrogenase |
| YGL244W | RTF1 | Nuclear protein |
| YCR012W | PGK1 | 3-phosphoglycerate kinase |
| YLR259C | HSP60 | mitochondrial chaperonin, homolog of E. |
| | | coli groEL protein |
| YPL106C | SSE1 | HSP70 family member, highly homologous |
| | | to Ssa1p and Sse2p |
| YMR116C | ASC1 | G-beta like protein |
| YDL229W | SSB1 | cytoplasmic member of the HSP70 family |
| YJL052W | TDH1 | Glyceraldehyde-3-phosphate |
| | | dehydrogenase 1 |
| YJR045C | SSC1 | Mitochondrial matrix protein involved in |
| | | protein import\; subunit of SceI |
| | | endonuclease |
| YKL060C | FBA1 | aldolase |
| YKL152C | GPM1 | Phosphoglycerate mutase |
| YBR072W | HSP26 | heat shock protein 26 |
| YMR186W | HSC82 | constitutively expressed heat shock protein |
| YER091C | MET6 | vitamin B12-(cobalamin)-independent |
| | | isozyme of methionine synthase (also called |
| | | N5-methyltetrahydrofolate homocysteine |
| | | methyltransferase or 5-methyltetra- |
| | | hydropteroyl triglutamate homocysteine |
| | | methyltransferase) |
| YBL075C | SSA3 | heat-inducible cytosolic member of the 70 |
| | | kDa heat shock protein family |
| YBR196C | PGII | Glucose-6-phosphate isomerase |
| YDR502C | SAM2 | S-adenosylmethionine synthetase |
| YDR099W | BMH2 | member of conserved eukaryotic 14-3-3 gene |
| | | family |
| YLR340W | RPP0 | 60S ribosomal protein P0 (A0) (L10E) |
| YGR214W | RPS0A | Ribosomal protein S0A |
| YLR180W | SAM1 | S-adenosylmethionine synthetase |
| YBRC19C | GAL10 | UDP-glucose 4-epimerase |
| YNL209W | SSB2 | Heat shock protein of HSP70 family, |
| | | homolog of SSB1 |
| YJR121W | ATP2 | F(1)F(0)-ATPase complex beta subunit, |
| | | mitochondrial |
| YOR308C | SNU66 | 66 kD U4\/U6.U5 snRNP associated protein |
| YJR009C | TDH2 | glyceraldehyde 3-phosphate dehydrogenase |
| YJL034W | KAR2 | Homologue of mammalian BiP (GPR78) |
| | | protein\; member of the HSP70 gene family |
| YMR108W | ILV2 | acetolactate synthase |
| YER177W | BMHI | Homolog of mammalian 14-3-3 proteins |
| YDR050C | TPI1 | triosephosphate isomerase |
| YBR127C | VMA2 | vacuolar ATPase V1 domain subunit B |
| | | (60 kDa) |
| YGR171C | MSM1 | mitochondrial methionyl-tRNA synthetase |
| YNL178W | RPS3 | Ribosomal protein S3 (rp13) (YS3) |
| YER043C | SAH1 | putative S-adenosyl-L-homocysteine |
| | | hydrolase |
| YLR355C | ILV5 | acetohydroxyacid reductoisomerase |
| YDR171W | HSP42 | heat shock protein similar to HSP26, |
| | | involved in cytoskeleton assembly |
| YHR020W | YHR020W | Aminoacyl tRNA-synthetase |
| YBR020W | GAL1 | galactokinase |
| YMR319C | FET4 | Low-affinity Fe(II) transport protein |
| YDL055C | PSA1 | mannose-1-phosphate guanyltransferase, |
| | | GDP-mannose pyrophosphorylase |
| YKL182W | FAS1 | pentafunctional enzyme consisting of the |
| | | following domains: acetyl transferase, enoyl |
| | | reductase, dehydratase and malonyl\/palmityl |
| | | transferase |
| YJR123W | RPS5 | Ribosomal protein S5 (S2) (rp14) (YS8) |
| YPL231W | FAS2 | alpha subunit of fatty acid synthase |
| YHR111W | YHR111W | moeB, thiF, UBA1 |
| YFR053C | HXK1 | Hexokinase I (PI) (also called Hexokinase A) |
| YKL104C | GFA1 | Glutamine_fructose-6-phosphate |
| | | amidotransferase (glucoseamine-6-phosphate |
| | | synthase) |
| YBL099W | ATP1 | mitochondrial F1F0-ATPase alpha subunit |
| YPL131W | RPL5 | Ribosomal protein L5 (L1a)(YL3) |
| YLR249W | YEF3 | EF-3 (translational elongation factor 3) |
| YER103W | SSA4 | member of 70 kDa heat shock protein family |
| YBR031W | RPL4A | Ribosomal protein L4A (L2A) (rp2) (YL2) |
| YOR375C | GDH1 | NADP-specific glutamate dehydrogenase |
| YDL126C | CDC48 | microsomal ATPase |
| YGL206C | CHC1 | presumed vesicle coat protein |
| YOR374W | ALD4 | mitochondrial aldehyde dehydrogenase |
| YFL039C | ACT1 | Actin |
| YHR203C | RPS4B | Ribosomal protein S4B (YS6) (rp5) (S7B) |
| YFR031C-A | RPL2A | Ribosomal protein L2A (L5A) (rp8) (YL6) |
| YLR048W | RPS0B | Ribosomal protein S0B |
| YGL048C | RPT6 | ATPase |
| YJL130C | URA2 | carbamoyl-phophate synthetase, aspartate |
| | | transcarbamylase, and glutamine |
| | | amidotransferase |
| YBR181C | RPS6B | 40S ribosomal gene product S6B (S10B) |
| | | (rp9) (YS4) |
| YDR394W | RPT3 | ATPase (AAA family) component of the 26S |
| | | proteasome complex |
| YJL008C | CCT8 | Component of Chaperonin Containing |
| | | T-complex subunit eight |
| YJL153C | INO1 | L-myo-inositol-1-phosphate synthase |
| YJL117W | PHO86 | Putative inorganic phosphate transporter |
| YOL145C | CTR9 | involved in mitosis and chromosome |
| | | segregation |
| YPL137C | YPL137C | |
| YGR240C | PFK1 | phosphofructokinase alpha subunit |
| YGL245W | YGL245W | |
| YBL039C | URA7 | CTP synthase, highly homologus to URA8 |
| | | CTP synthase |
| YGL008C | PMA1 | plasma membrane H+-ATPase |
| YER110C | KAP123 | Karyopherin beta 4 |
| YGL253W | HXK2 | Hexokinase II (PII) (also called |
| | | Hexokinase B) |
| YHR199C | YHR199C | |
| YDR385W | EFT2 | translation elongation factor 2 (EF-2) |
| YJL138C | TIF2 | translation initiation factor eIF4A |
| YPL061W | ALD6 | Cytosolic Aldehyde Dehydrogenase |
| YHR137W | ARO9 | aromatic amino acid aminotransferase II |
| YML028W | TSA1 | thioredoxin-peroxidase (TPx)\; reduces |
| | | H2O2 and alkyl hydroperoxides with the use |
| | | of hydrogens provided by thioredoxin, |
| | | thioredoxin reductase, and NADPH |
| YMR257C | PET111 | translational activator of cytochrome c |
| | | oxidase subunit II |
| YOR136W | IDH2 | NAD+-dependent isocitrate dehydrogenase |
| YOR117W | RPT5 | 26S protease regulatory subunit |
| YFL037W | TUB2 | beta-tubulin |
| YOR063W | RPL3 | Ribosomal protein L3 (rp1) (YL1) |
| YNL037C | IDH1 | alpha-4-beta-4 subunit of mitochondrial |
| | | isocitrate dehydrogenase 1 |
| YCL043C | PDI1 | protein disulfide isomerase |
| YML063W | RPS1B | Ribosomal protein S1B (rp10B) |
| YBR018C | GAL7 | galactose-1-phosphate uridyl transferase |
| YJR077C | MIR1 | |
| YCL061C | MRC1 | |
| YLR134W | PDC5 | pyruvate decarboxylase |
| YHL033C | RPL8A | Ribosomal protein L8A (rp6) (YL5) (L4A) |
| YDR012W | RPL4B | Ribosomal protein L4B (L2B) (rp2) (YL2) |
| YPL240C | HSP82 | 82 kDa heat shock protein\; homolog of |
| | | mammalian Hsp90 |
| YBL072C | RPS8A | Ribosomal protein S8A (S14A) (rp19) (YS9) |
| YDL083C | RPS16B | Ribosomal protein S16B (rp61R) |
| YJR109C | CPA2 | carbamyl phosphate synthetase |
| YGL076C | RPL7A | Ribosomal protein L7A (L6A) (rp11) (YL8) |
| YLR304C | ACO1 | Aconitase, mitochondrial |
| YDL143W | CCT4 | component of chaperonin complex |
| YDL185W | TFP1 | vacuolar ATPase V1 domain subunit A |
| | | (69 kDa) |
| YOR123C | LEO1 | |
| YOR096W | RPS7A | Ribosomal protein S7A (rp30) |
| YGR094W | VAS1 | mitochondrial and cytoplasmic valyl-tRNA |
| | | synthetase |
| YBR169C | SSE2 | HSP70 family member, highly homologous |
| | | to Sse1p |
| YBR011C | IPP1 | Inorganic pyrophosphatase |
| YDR018C | YDR018C | |
| YKL035W | UGP1 | Uridinephosphoglucose pyrophosphorylase |
| YFR030W | MET10 | subunit of assimilatory sulfite reductase |
| YKL081W | TEF4 | Translation elongation factor EF-1gamma |
| YJR104C | SOD1 | Cu, Zn superoxide dismutase |
| YHL015W | RPS20 | Ribosomal protein S20 |
| YPL258C | THI21 | THI for thiamine metabolism. Transcribed in |
| | | the presence of low level of thiamine |
| | | (10-8M) and turned off in the presence of |
| | | high level (10-6M) of thiamine. Under the |
| | | positive control of THI2 and THI3. |
| YHR027C | RPN1 | Subunit of 26S Proteasome (PA700 subunit) |
| YNL055C | POR1 | Outer mitochondrial membrane porin |
| | | (voltage-dependent anion channel, or |
| | | VDAC) |
| YLR441C | RPS1A | Ribosomal protein S1A (rp10A) |
| YLR354C | TAL1 | Transaldolase, enzyme in the pentose |
| | | phosphate pathway |
| YGL062W | PYC1 | pyruvate carboxylase |
| YDR190C | RVB1 | RUVB-like protein |
| YCL040W | GLK1 | Glucokinase |
| YBL021C | HAP3 | transcriptional activator protein of CYC1 |
| YBR218C | PYC2 | pyruvate carboxylase |
| YLR058C | SHM2 | serine hydroxymethyltransferase |
| YDR477W | SNF1 | protein serine\/threonine kinase |
| YGR085C | RPL11B | 60S ribosomal protein L11B (L16B) |
| | | (rp39B) (YL22) |
| YDR158W | HOM2 | aspartic beta semi-aldehyde dehydrogenase |
| YPR159W | KRE6 | potential beta-glucan synthase |
| YIL107C | PFK26 | 6-Phosphofructose-2-kinase |
| YGL234W | ADE5, 7 | glycinamide ribotide synthetase and |
| | | aminoimidazole ribotide synthetase |
| YOR369C | RPS12 | 40S ribosomal protein S12 |
| YMR247C | YMR247C | |
| YBR189W | RPS9B | Ribosomal protein S9B (S13) (rp21) (YS11) |
| YLL026W | HSP104 | 104 kDa heat shock protein |
| YDL007W | RPT2 | (putative) 26S protease subunit |
| YOR261C | RPN8 | Subunit of the regulatory particle of the |
| | | proteasome |
| YIL142W | CCT2 | molecular chaperone |
| YFL045C | SEC53 | phosphomannomutase |
| YNL064C | YDJ1 | yeast dnaJ homolog (nuclear envelope |
| | | protein)\; heat shock protein |
| YKL022C | CDC16 | putative metal-binding nucleic acid-binding |
| | | protein, interacts with Cdc23p and Cdc27p to |
| | | catalyze the conjugation of ubiquitin to |
| | | cyclin B |
| YNL040W | YNL040W | |
| YML085C | TUB1 | alpha-tubulin |
| YIL033C | BCY1 | regulatory subunit of cAMP-dependent |
| | | protein kinase |
| YGR180C | RNR4 | Ribonucleotide Reductase |
| YDR064W | RPS13 | Ribosomal protein S13 (S27a) (YS15) |
| YCR002C | CDC10 | conserved potential GTP-ginding protein |
| YML124C | TUB3 | alpha-tubulin |
| YIL094C | LYS12 | Homo-isocitrate dehydrogenase |
| YLR153C | ACS2 | acetyl-coenzyme A synthetase |
| YPR074C | TKL1 | Transketolase 1 |
| YDR212W | TCP1 | chaperonin subunit alpha |
| YDR155C | CPH1 | cyclophilin peptidyl-prolyl cis-trans |
| | | isomerase |
| YHR183W | GND1 | Phosphogluconate Dehydrogenase |
| | | (Decarboxylating) |
| YJR139C | HOM6 | Homoserine dehydrogenase |
| | | (L-homoserine:NADP oxidoreductase) |
| YER062C | HOR2 | DL-glycerol-3-phosphatase |
| YJR064W | CCT5 | subunit of chaperonin subunit epsilon |
| YDR447C | RPS17B | Ribosomal protein S17B (rp51B) |
| YGL026C | TRP5 | tryptophan synthetase |
| YOL139C | CDC33 | mRNA cap binding protein eIF-4E |
| YDR450W | RPS18A | Ribosomal protein S18A |
| YER074W | RPS24A | 40S ribosomal protein S24A |
| YNR001C | CIT1 | citrate synthase. Nuclear encoded |
| | | mitochondrial protein. |
| YDL082W | RPL13A | Ribosomal protein LI3A |
| YLR150W | STM1 | gene product has affinity for quadruplex |
| | | nucleic acids |
| YGL147C | RPL9A | Ribosomal protein L9A (L8A) (rp24) (YL11) |
| YBR025C | YBR025C | probable purine nucleotide-binding protein |
| YGL135W | RPL1B | Ribosomal protein L1B |
| YGL105W | ARC1 | G4 nucleic acid binding protein, involved in |
| | | tRNA aminoacylation |
| YHR179W | OYE2 | NAPDH dehydrogenase (old yellow |
| | | enzyme), isoform 2 |
| YDL182W | LYS20 | homocitrate synthase, highly homologous |
| | | to YDL131W |
| YJL066C | MPM1 | |
| YBR279W | PAF1 | RNA polymerase II-associated protein |
| YIL053W | RHR2 | DL-glycerol-3-phosphatase |
| YEL051W | VMA8 | vacuolar ATPase V1 domain subunit D |
| YMR205C | PFK2 | phosphofructokinase beta subunit |
| YMR120C | ADE17 | 5-aminoimidazole-4-carboxamide |
| | | ribonucleotide (AICAR) |
| | | transformylase\/IMP cyclohydrolase |
| YDR148C | KGD2 | dihydrolipoyl tranasuccinylase component |
| | | of alpha-ketoglutarate dehydrogenase |
| | | complex in mitochondria |
| YMR145C | YMR145C | |
| YNR058W | BIO3 | 7,8-diamino-pelargonic acid |
| | | aminotransferase (DAPA) aminotransferase |
| YCR084C | TUP1 | glucose repression regulatory protein, |
| | | exhibits similarity to beta subunits of G |
| | | proteins |
| YLL045C | RPL8B | Ribosomal protein L8B (L4B) (rp6) (YL5) |
| YLL018C | DPS1 | Aspartyl-tRNA synthetase, cytosolic |
| YGL202W | ARO8 | aromatic amino acid aminotransferase |
| YBL076C | ILS1 | cytoplasmic isoleucyl-tRNA synthetase |
| YLR109W | AHP1 | alkyl hydroperoxide reductase |
| YDR279W | YDR279W | |
| YPL110C | YPL110C | |
| YKL210W | UBA1 | ubiquitin activating enzyme, similar to |
| | | Uba2p |
| YPL235W | RVB2 | RUVB-like protein |
| YMR226C | YMR226C | |
| YBR126C | TPS1 | 56 kD synthase subunit of trehalose-6- |
| | | phosphate synthase\/phosphatase complex |
| YLR075W | RPL10 | Ribosomal protein L10\; Ubiquinol- |
| | | cytochrome C reductase complex subunit VI |
| | | requiring protein |
| YGR155W | CYS4 | Cystathionine beta-synthase |
| YDR427W | RPN9 | Subunit of the regulatory particle of the |
| | | proteasome |
| YOR317W | FAA1 | long chain fatty acyl:CoA synthetase |
| YJR105W | ADO1 | adenosine kinase |
| YLR438W | CAR2 | ornithine aminotransferase |
| YBR121C | GRS1 | Glycyl-tRNA synthase |
| YLR222C | YLR222C | |
| YMR315W | YMR315W | |
| YER021W | RPN3 | component of the regulatory module of the |
| | | 26S proteasome, homologous to human p58 |
| | | subunit |
| YGL256W | ADH4 | alcohol dehydrogenase isoenzyme IV |
| YMR105C | PGM2 | Phosphoglucomutase |
| YMR062C | ECM40 | acetylornithine acetyltransferase |
| YPL028W | ERG10 | acetoacetyl CoA thiolase |
| YER178W | PDA1 | alpha subunit of pyruvate dehydrogenase |
| | | (E1 alpha) |
| YCR031C | RPS14A | Ribosomal protein SL4A (rp59A) |
| YOR259C | RPT4 | ATPase\; component of the 26S proteasome |
| | | cap subunit |
| YJL167W | ERG20 | Farnesyl diphosphate synthetase |
| | | (FPP synthetase) |
| YDL124W | YDL124W | |
| YAR010C | YAR010C | TY1B |
| YDL225W | SHS1 | Septin homolog |
| YFR004W | RPN11 | Similar to S. pombe PAD1 gene product |
| YOR151C | RPB2 | second largest subunit of RNA polymerase II |
| YOL058W | ARG1 | arginosuccinate synthetase |
| YNL302C | RPS19B | Ribosomal protein S19B (rp55B) (S16aB) |
| | | (YS16B) |
| YBR048W | RPS11B | Ribosomal protein S11B (S18B) (rp41B) |
| | | (YS12) |
| YNL069C | RPL16B | Ribosomal protein LL6B (L2LB) (rp23) |
| | | (YL15) |
| YPR191W | QCR2 | 40 kDa ubiquinol cytochrome-c reductase |
| | | core protein 2 |
| YDR471W | RPL27B | Ribosomal protein L27B |
| YCR053W | THR4 | threonine synthase |
| YGL123W | RPS2 | Ribosomal protein S2 (S4) (rp12) |
| | | (YS5) |
| YJL026W | RNR2 | small subunit of ribonucleotide reductase |
| YOL138C | YOL138C | |
| YJR070C | YJR070C | |
| YBL027W | RPL19B | Ribosomal protein L19B (YL14) (L23B) |
| | | (rp15L) |
| YBR221C | PDB1 | beta subunit of pyruvate dehydrogenase |
| | | (E1 beta) |
| YDR127W | ARO1 | pentafunctional arom polypeptide (contains: |
| | | 3-dehydroquinate synthase, 3-dehydroquinate |
| | | dehydratase (3-dehydroquinase), shikimate |
| | | 5-dehydrogenase, shikimate kinase, and |
| | | epap synthase) |
| YDL097C | RPN6 | Subunit of the regulatory particle of the |
| | | proteasome |
| YEL060C | PRB1 | vacuolar protease B |
| YDR418W | RPL12B | Ribosomal protein LL2B (L15B) (YL23) |
| YLR448W | RPL6B | 60S ribosomal subunit protein L6B (L17B) |
| | | (rp18) (YL16) |
| YDR129C | SAC6 | fibrim homolog (actin-filament bundling |
| | | protein) |
| YHR082C | KSP1 | Ser\/Thr protein kinase |
| YDR342C | HXT7 | Hexose transporter |

TABLE 5A
Proteins identified in control lanes (Protocol A).

| ORF Name | Gene | Description |
|---|---|---|
| YOL086C | ADH1 | Alcohol dehydrogenase |
| YMR116C | ASC1 | G-beta like protein |
| YAL038W | CDC19 | Pyruvate kinase |
| YLR418C | CDC73 | RNA polymerase II accessory protein |
| YOL145C | CTR9 | involved in mitosis and chromosome |
| | | segregation |
| YDR385W | EFT2 | translation elongation factor 2 (EF-2) |
| YGR254W | ENO1 | enolase I |
| YHR174W | ENO2 | enolase |
| YPL231W | FAS2 | alpha subunit of fatty acid synthase |
| YKL060C | FBA1 | aldolase |
| YMR186W | HSC82 | constitutively expressed heat shock |
| | | protein |
| YGL253W | HXK2 | Hexokinase II (PII) (also called Hexokinase B) |
| YDR499W | LCD1 | |
| YOR123C | LEO1 | |
| YGL009C | LEU1 | isopropylmalate isomerase |
| YBR136W | MEC1 | similar to phosphatidylinositol(PI)3- |
| | | kinases required for DNA damage |
| | | induced checkpoint responses in G1, |
| | | S\/M, intra S, and G2\/M in mitosis |
| YBR279W | PAF1 | RNA polymerase II-associated protein |
| YBR221C | PDB1 | beta subunit of pyruvate dehydrogenase |
| | | (E1 beta) |
| YLR044C | PDC1 | pyruvate decarboxylase |
| YMR076C | PDS5 | (putative) involved in sister |
| | | chromosome cohesion during mitosis |
| YBR035C | PDX3 | pyridoxine (pyridoxiamine) phosphate |
| | | oxidase |
| YIL107C | PFK26 | 6-Phosphofructose-2-kinase |
| YCR012W | PGK1 | 3-phosphoglycerate kinase |
| YAR007C | RFA1 | 69 kDa subunit of the heterotrimeric |
| | | RPA (RF-A) single-stranded DNA |
| | | binding protein, binds URS1 and |
| | | CAR1 |
| YNL312W | RFA2 | subunit 2 of replication factor RF-A\; |
| | | 29\% identical to the human p34 subunit |
| | | of RF-A |
| YJL173C | RFA3 | subunit 3 of replication factor-A |
| YCR028C-A | RIM1 | Single-stranded zinc finger DNA- |
| | | binding protein |
| YGR180C | RNR4 | Ribonucleotide Reductase |
| YJL148W | RPA34 | unshared RNA polymerase I subunit |
| YPR102C | RPL11A | Ribosomal protein L11A (L16A) |
| | | (rp39A) (YL22) |
| YGR085C | RPL11B | 60S ribosomal protein L11B (L16B) |
| | | (rp39B) (YL22) |
| YDR418W | RPL12B | Ribosomal protein L12B (L15B) |
| | | (YL23) |
| YDL082W | RPL13A | Ribosomal protein L13A |
| YNL069C | RPL16B | Ribosomal protein L16B (L21B) |
| | | (rp23) (YL15) |
| YKL180W | RPL17A | Ribosomal protein L17A (L20A) |
| | | (YL17) |
| YJL177W | RPL17B | Ribosomal protein L17B (L20B) |
| | | (YL17) |
| YBL027W | RPL19B | Ribosomal protein L19B (YL14) |
| | | (L23B) (rp15L) |
| YGL135W | RPL1B | Ribosomal protein L1B |
| YMR242C | RPL20A | Ribosomal protein L20A (L18A) |
| YOR312C | RPL20B | 60S ribosomal protein L20B (L18B) |
| YBR191W | RPL21A | Ribosomal protein L21A |
| YPL079W | RPL21B | Ribosomal protein L21B |
| YBL087C | RPL23A | Ribosomal protein L23A (L17aA) |
| | | (YL32) |
| YOL127W | RPL25 | Ribosomal protein L25 (rp16L) |
| | | (YL25) |
| YHR010W | RPL27A | Ribosomal protein L27A |
| YDR471W | RPL27B | Ribosomal protein L27B |
| YIL018W | RPL2B | Ribosomal protein L2B (L5B) (rp8) |
| | | (YL6) |
| YOR063W | RPL3 | Ribosomal protein L3 (rp1) (YL1) |
| YGL030W | RPL30 | Large ribosomal subunit protein L30 |
| | | (L32) (rp73) (YL38) |
| YPL143W | RPL33A | Ribosomal protein L33A (L37A) |
| | | (YL37) (rp47) |
| YDL191W | RPL35A | Ribosomal protein L35A |
| YJR094W-A | RPL43B | Ribosomal protein L43B |
| YBR031W | RPL4A | Ribosomal protein L4A (L2A) (rp2) |
| | | (YL2) |
| YDR012W | RPL4B | Ribosomal protein LAB (L2B) (rp2) |
| | | (YL2) |
| YPL131W | RPL5 | Ribosomal protein L5 (L1a) (YL3) |
| YML073C | RPL6A | Ribosomal protein L6A (L17A) (rp18) |
| | | (YL16) |
| YLR448W | RPL6B | 60S ribosomal subunit protein L6B |
| | | (L17B) (rp18) (YL16) |
| YGL076C | RPL7A | Ribosomal protein L7A (L6A) (rp11) |
| | | (YL8) |
| YPL198W | RPL7B | Ribosomal protein L7B (L6B) (rp11) |
| | | (YL8) |
| YHL033C | RPL8A | Ribosomal protein L8A (rp6) (YL5) |
| | | (L4A) |
| YGL147C | RPL9A | Ribosomal protein L9A (L8A) (rp24) |
| | | (YL11) |
| YLR340W | RPP0 | 60S ribosomal protein P0 (A0) (L10E) |
| YGR214W | RPS0A | Ribosomal protein S0A |
| YLR048W | RPS0B | Ribosomal protein S0B |
| YOR293W | RPS10A | Ribosomal protein S10A |
| YBR048W | RPS11B | Ribosomal protein S11B (S18B) |
| | | (rp41B) (YS12) |
| YOR369C | RPSL2 | 40S ribosomal protein S12 |
| YDR064W | RPS13 | Ribosomal protein S13 (S27a) (YS15) |
| YOL040C | RPS15 | 40S ribosomal protein S15 (S21) (rp52) |
| | | (RIG protein) |
| YDL083C | RPS16B | Ribosomal protein S16B (rp61R) |
| YML024W | RPS17A | Ribosomal protein S17A (rp51A) |
| YDR447C | RPS17B | Ribosomal protein S17B (rp51B) |
| YDR450W | RPS18A | Ribosomal protein S18A |
| YLR441C | RPS1A | Ribosomal protein S1A (rp10A) |
| YML063W | RPS1B | Ribosomal protein S1B (rp10B) |
| YGL123W | RPS2 | Ribosomal protein S2 (S4) (rp12) (YS5) |
| YHL015W | RPS20 | Ribosomal protein S20 |
| YJL190C | RPS22A | Ribosomal protein S22A (S24A) (rp50) |
| | | (YS22) |
| YER074W | RPS24A | 40S ribosomal protein S24A |
| YGR027C | RPS25A | Ribosomal protein S25A (S31A) (rp45) |
| | | (YS23) |
| YNL178W | RPS3 | Ribosomal protein S3 (rp13) (YS3) |
| YHR203C | RPS4B | Ribosomal protein S4B (YS6) (rp5) |
| | | (S7B) |
| YJR123W | RPS5 | Ribosomal protein S5 (S2) (rp14) |
| | | (YS8) |
| YPL090C | RPS6A | Ribosomal protein S6A (S10A) (rp9) |
| | | (YS4) |
| YBR181C | RPS6B | 40S ribosomal gene product S6B |
| | | (S10B) (rp9) (YS4) |
| YBL072C | RPS8A | Ribosomal protein S8A (S14A) (rp19) |
| | | (YS9) |
| YPL081W | RPS9A | Ribosomal protein S9A (S13) (rp21) |
| | | (YS11) |
| YGL244W | RTF1 | Nuclear protein |
| YAL005C | SSA1 | Heat shock protein of HSP70 family, |
| | | cytoplasmic |
| YLL024C | SSA2 | member of 70 kDa heat shock protein |
| | | family |
| YBL075C | SSA3 | heat-inducible cytosolic member of the |
| | | 70 kDa heat shock protein family |
| YER103W | SSA4 | member of 70 kDa heat shock protein |
| | | family |
| YDL229W | SSB1 | cytoplasmic member of the HSP70 |
| | | family |
| YNL209W | SSB2 | Heat shock protein of HSP70 family, |
| | | homolog of SSB1 |
| YPL106C | SSE1 | HSP70 family member, highly |
| | | homologous to Ssa1p and Sse2p |
| YBR169C | SSE2 | HSP70 family member, highly |
| | | homologous to Sse1p |
| YLR150W | STM1 | gene product has affinity for quadruplex |
| | | nucleic acids |
| YJL052W | TDH1 | Glyceraldehyde-3-phosphate |
| | | dehydrogenase 1 |
| YJR009C | TDH2 | glyceraldehyde 3-phosphate |
| | | dehydrogenase |
| YGR192C | TDH3 | Glyceraldehyde-3-phosphate |
| | | dehydrogenase 3 |
| YDR050C | TPI1 | triosephosphate isomerase |
| YML028W | TSA1 | thioredoxin-peroxidase (TPx)\; |
| | | reduces H2O2 and alkyl hydroperoxides |
| | | with the use of hydrogens provided by |
| | | thioredoxin, thioredoxin reductase, |
| | | and NADPH |
| YBR012W-B | YBR012W-B | The TyB Gag-Pol protein. Gag |
| | | processing produces capsid proteins. |
| | | Pol is cleaved to produce protease, |
| | | reverse transcriptase, and integrase |
| | | activities. |
| YDR210W-D | YDR210W-D | The TyB Gag-Pol protein. Gag |
| | | processing produces capsid proteins. |
| | | Pol is cleaved to produce protease, |
| | | reverse transcriptase, and integrase |
| | | activities. |
| YDR261C-C | YDR261C-C | TyA gag protein. Gag processing |
| | | produces capsid proteins. |
| YDR261C-D | YDR261C-D | The TyB Gag-Pol protein. Gag |
| | | processing produces capsid proteins. |
| | | Pol is cleaved to produce protease, |
| | | reverse transcriptase, and integrase |
| | | activities. |
| YDR316W-B | YDR316W-B | The TyB Gag-Pol protein. Gag |
| | | processing produces capsid proteins. |
| | | Pol is cleaved to produce protease, |
| | | reverse transcriptase, and integrase |
| | | activities. |
| YDR365W-B | YDR365W-B | The TyB Gag-Pol protein. Gag |
| | | processing produces capsid proteins. |
| | | Pol is cleaved to produce protease, |
| | | reverse transcriptase, and integrase |
| | | activities. |
| YLR249W | YEF3 | EF-3 (translational elongation factor 3) |
| YGR027W-B | YGR027W-B | The TyB Gag-Pol protein. Gag |
| | | processing produces capsid proteins. |
| | | Pol is cleaved to produce protease, |
| | | reverse transcriptase, and integrase |
| | | activities. |
| YHR111W | YHR111W | moeB, thiF, UBA1 |
| YJR029W | YJR029W | |
| YMR247C | YMR247C | |
| YNL054W-A | YNL054W-A | TyA Gag protein. Gag processing |
| | | produces capsid proteins. |
| YOR142W-B | YOR142W-B | TyB Gag-Pol protein. Gag processing |
| | | produces capsid proteins. Pol is cleaved |
| | | to produce protease, reverse |
| | | transcriptase and integrase activities. |
| YPL137C | YPL137C | |
| YPL257W-B | YPL257W-B | TyB Gag-Pol protein. Gag processing |
| | | produces capsid proteins. Pol is cleaved |
| | | to produce protease, reverse transcriptase |
| | | and integrase activities. |

TABLE 5B
Proteins identified in control lanes (Protocol B)

| ORF Name | Gene | Description |
|---|---|---|
| YOL086C | ADH1 | Alcohol dehydrogenase |
| YMR116C | ASC1 | G-beta like protein |
| YBL099W | ATP1 | mitochondrial F1F0-ATPase alpha subunit |
| YJR121W | ATP2 | F(1)F(0)-ATPase complex beta subunit, |
| | | mitochondrial |
| YIL142W | CCT2 | molecular chaperone |
| YJL014W | CCT3 | Cytoplasmic chaperonin subunit gamma |
| YDL143W | CCT4 | component of chaperonin complex |
| YJR064W | CCT5 | subunit of chaperonin subunit epsilon |
| YJL111W | CCT7 | Component of Chaperonin Containing |
| | | T-complex subunit seven |
| YJL008C | CCT8 | Component of Chaperonin Containing |
| | | T-complex subunit eight |
| YKL022C | CDC16 | putative metal-binding nucleic |
| | | acid-binding protein, |
| | | interacts with Cdc23p and Cdc27p to |
| | | catalyze the conjugation of ubiquitin |
| | | to cyclin B |
| YAL038W | CDCL9 | Pyruvate kinase |
| YLR418C | CDC73 | RNA polymerase II accessory protein |
| YGR218W | CRM1 | omosome region maintenance protein |
| YOL145C | CTR9 | involved in mitosis and chromosome |
| | | segregation |
| YHR174W | ENO2 | enolase |
| YK1182W | FAS1 | pentafunctional enzyme consisting of |
| | | the following domains: acetyl transferase, |
| | | enoyl reductase, dehydratase and |
| | | malonyl\/palmityl transferase |
| YPL231W | FAS2 | alpha subunit of fatty acid synthase |
| YMR319C | FET4 | Low-affinity Fe(II) transport protein |
| YGR267C | FOL2 | GTP-cyclohydrolase I |
| YKL152C | GPM1 | Phosphoglycerate mutase |
| YER110C | KAPL23 | Karyopherinbeta4 |
| YPR159W | KRE6 | potential beta-glucan synthase |
| YHR082C | KSP1 | Ser\/Thr protein kinase |
| YNL071W | LAT1 | Dihydrolipoamide acetyltransferase |
| | | component (E2) of pyruvate dehydrogenase |
| | | complex |
| YDR499W | LCD1 | |
| YORL23C | LEO1 | |
| YGL009C | LEU1 | isopropylmalate isomerase |
| YFR001W | LOC1 | Double-stranded RNA-binding protein |
| YBR136W | MEC1 | similar to phosphatidylinositol(PI)3-kinases |
| | | required for DNA damage induced |
| | | checkpoint responses in G1, S\/M, intra S, |
| | | and G2\/M in mitosis |
| YDL167C | NRP1 | Asparagine-rich protein |
| YDR356W | NUF1 | component of the spindle pole body that |
| | | interacts with Spc42p, calmodulin, and a 35 |
| | | kDa protein |
| YBR279W | PAF1 | RNA polymerase II-associated protein |
| YBR221C | PDB1 | beta subunit of pyruvate dehydrogenase |
| | | (E1 beta) |
| YMR076C | PDS5 | (putative) involved in sister chromosome |
| | | cohesion during mitosis |
| YIL107C | PFK26 | 6-Phosphofructose-2-kinase |
| YCR012W | PGK1 | 3-phosphoglycerate kinase |
| YNL055C | POR1 | Outer mitochondrial membrane porin |
| | | (voltage-dependent anion channel, or |
| | | VDAC) |
| YDL055C | PSA1 | mannose-1-phosphate guanyltransferase, |
| | | GDP-mannose pyrophosphorylase |
| Q0255 | Q0255 | |
| YJL173C | RFA3 | subunit 3 of replication factor-A |
| YCR028C-A | RIM1 | Single-stranded zinc finger DNA-binding |
| | | protein |
| YJL148W | RPA34 | unshared RNA polymerase I subunit |
| YOR151C | RPB2 | second largest subunit of RNA polymerase II |
| YLR075W | RPL10 | Ribosomal protein L10\; Ubiquinol- |
| | | cytochrome C reductase complex subunit VI |
| | | requiring protein |
| YGR085C | RPL11B | 60S ribosomal protein L11B (L16B) (rp39B) |
| | | (YL22) |
| YDR418W | RPL12B | Ribosomal protein L12B (L15B) (YL23) |
| YMR142C | RPL13B | Ribosomal protein L13B |
| YHL001W | RPL14B | Ribosomal protein L14B |
| YLR029C | RPL15A | Ribosomal protein L15A (YL10) (rp15R) |
| | | (L13A) |
| YIL133C | RPL16A | Ribosomal protein L16A (L21A) (rp22) |
| | | (YL15) |
| YNL069C | RPL16B | Ribosomal protein L16B (L21B) (rp23) |
| | | (YL15) |
| YKL180W | RPL17A | Ribosomal protein L17A (L20A) (YL17) |
| YJL177W | RPL17B | Ribosomal protein L17B (L20B) (YL17) |
| YNL301C | RPL18B | Ribosomal protein L18B (rp28B) |
| YBL027W | RPL19B | Ribosomal protein L19B (YL14) (L23B) |
| | | (rp15L) |
| YGL135W | RPL1B | Ribosomal protein L1B |
| YMR242C | RPL20A | Ribosomal protein L20A (L18A) |
| YBR191W | RPL21A | Ribosomal protein L21A |
| YLR061W | RPL22A | Ribosomal protein L22A (L1c) (rp4) (YL31) |
| YGR148C | RPL24B | Ribosomal protein L24B (rp29) (YL21) |
| | | (L30B) |
| YLR344W | RPL26A | Ribosomal protein L26A (L33A) (YL33) |
| YFR031C-A | RPL2A | Ribosomal protein L2A (L5A) (rp8) (YL6) |
| YOR063W | RPL3 | Ribosomal protein L3 (rp1) (YL1) |
| YGL030W | RPL30 | Large ribosomal subunit protein L30 (L32) |
| | | (rp73) (YL38) |
| YDL075W | RPL31A | Ribosomal protein L31A (L34A) (YL28) |
| YBL092W | RPL32 | Ribosomal protein L32 |
| YPL143W | RPL33A | Ribosomal protein L33A (L37A) (YL37) |
| | | (rp47) |
| YOR234C | RPL33B | Ribosomal protein L33B (L37B) (rp47) |
| | | (YL37) |
| YDL191W | RPL35A | Ribosomal protein L35A |
| YMR194W | RPL36A | Ribosomal protein L36A (L39) (YL39) |
| YJR094W-A | RPL43B | Ribosomal protein L43B |
| YBR031W | RPL4A | Ribosomal protein L4A (L2A) (rp2) (YL2) |
| YDR012W | RPL4B | Ribosomal protein L4B (L2B) (rp2) (YL2) |
| YPL13LW | RPL5 | Ribosomal protein L5 (L1a) (YL3) |
| YML073C | RPL6A | Ribosomal protein L6A (L17A) (rp18) |
| | | (YL16) |
| YLR448W | RPL6B | 60S ribosomal subunit protein L6B (L17B) |
| | | (rp18) (YL16) |
| YPL198W | RPL7B | Ribosomal protein L7B (L6B) (rp11) (YL8) |
| YHL033C | RPL8A | Ribosomal protein L8A (rp6) (YL5) (L4A) |
| YLL045C | RPL8B | Ribosomal protein L8B (L4B) (rp6) (YL5) |
| YGL147C | RPL9A | Ribosomal protein L9A (L8A) (rp24) (YL11) |
| YHR027C | RPN1 | Subunit of 26S Proteasome (PA700 subunit) |
| YHR200W | RPN10 | homolog of the mammalian S5a protein, |
| | | component of 26S proteasome |
| YFR004W | RPN11 | Similar to S. pombe PAD1 gene product |
| YFR052W | RPN12 | cytoplasmic 32-34 kDa protein |
| YIL075C | RPN2 | RPN2p is a component of the 26S |
| | | proteosome |
| YER021W | RPN3 | component of the regulatory module of the |
| | | 26S proteasome, homologous to human p58 |
| | | subunit |
| YDL147W | RPN5 | Subunit of the regulatory particle of the |
| | | proteasome |
| YDL097C | RPN6 | Subunit of the regulatory particle of the |
| | | proteasome |
| YPR108W | RPN7 | Subunit of the regulatory particle of the |
| | | proteasome |
| YOR261C | RPN8 | Subunit of the regulatory particle of the |
| | | proteasome |
| YDR427W | RPN9 | Subunit of the regulatory particle of the |
| | | proteasome |
| YLR340W | RPP0 | 60S ribosomal protein P0 (A0) (L10E) |
| YOL039W | RPP2A | 60S acidic ribosomal protein P2A (L44) |
| | | (A2) (YP2alpha) |
| YDR382W | RPP2B | Ribosomal protein P2B (YP2beta) (L45) |
| YGR214W | RPS0A | Ribosomal protein S0A |
| YOR293W | RPS10A | Ribosomal protein S10A |
| YBR048W | RPS11B | Ribosomal protein S11B (S18B) (rp41B) |
| | | (YS12) |
| YOR369C | RPS12 | 40S ribosomal protein S12 |
| YDR064W | RPS13 | Ribosomal protein S13 (S27a) (YS15) |
| YCR031C | RPS14A | Ribosomal protein S14A (rp59A) |
| YOL040C | RPS15 | 40S ribosomal protein S15 (S21) (rp52) |
| | | (RIG protein) |
| YDL083C | RPS16B | Ribosomal protein S16B (rp61R) |
| YDR447C | RPS17B | Ribosomal protein S17B (rp51B) |
| YDR450W | RPS18A | Ribosomal protein S18A |
| YNL302C | RPS19B | Ribosomal protein S19B (rp55B) (S16aB) |
| | | (YS16B) |
| YLR441C | RPS1A | Ribosomal protein S1A (rp10A) |
| YML063W | RPS1B | Ribosomal protein S1B (rp10B) |
| YJL190C | RPS22A | Ribosomal protein S22A (S24A) |
| | | (rp50) (YS22) |
| YLR367W | RPS22B | Ribosomal protein S22B (S24B) |
| | | (rp50) (YS22) |
| YER074W | RPS24A | 40S ribosomal protein S24A |
| YGR027C | RPS25A | Ribosomal protein S25A (S31A) |
| | | (rp45) (YS23) |
| YNL178W | RPS3 | Ribosomal protein S3 (rp13) (YS3) |
| YLR287C-A | RPS30A | Ribosomal protein S30A |
| YHR203C | RPS4B | Ribosomal protein S4B (YS6) (rp5) |
| | | (S7B) |
| YJR123W | RPS5 | Ribosomal protein S5 (S2) (rp14) (YS8) |
| YBR181C | RPS6B | 40S ribosomal gene product S6B (S10B) |
| | | (rp9) (YS4) |
| YOR096W | RPS7A | Ribosomal protein S7A (rp30) |
| YNL096C | RPS7B | Ribosomal protein S7B (rp30) |
| YBL072C | RPS8A | Ribosomal protein S8A (S14A) |
| | | (rp19) (YS9) |
| YPL081W | RPS9A | Ribosomal protein S9A (S13) (rp21) (YS11) |
| YBR189W | RPS9B | Ribosomal protein S9B (S13) (rp21) (YS11) |
| YKL145W | RPT1 | putative ATPase, 26S protease subunit |
| | | component |
| YDL007W | RPT2 | (putative) 26S protease subunit |
| YOR117W | RPT5 | 26S protease regulatory subunit |
| YGL048C | RPT6 | ATPase |
| YGL244W | RTF1 | Nuclear protein |
| YLR180W | SAM1 | S-adenosylmethionine synthetase |
| YDR502C | SAM2 | S-adenosylmethionine synthetase |
| YAL005C | SSA1 | Heat shock protein of HSP70 family, |
| | | cytoplasmic |
| YLL024C | SSA12 | member of 70 kDa heat shock protein |
| | | family |
| YNL209W | SSB2 | Heat shock protein of HSP70 family, |
| | | homolog of SSB1 |
| YPL106C | SSE1 | HSP70 family member, highly homologous |
| | | to Ssa1p and Sse2p |
| YLR150W | STM1 | gene product has affinity for quadruplex |
| | | nucleic acids |
| YDR212W | TCP1 | chaperonin subunit alpha |
| YGR192C | TDH3 | Glyceraldehyde-3-phosphate |
| | | dehydrogenase 3 |
| YBR118W | TEF2 | translational elongation factor EF-1 alpha |
| YOL055C | THI20 | THI for thiamine metabolism. Transcribed in |
| | | the presence of low level of thiamine |
| | | (10-8M) and turned off in the presence of |
| | | high level (10-6M) of thiamine. Under the |
| | | positive control of THI2 and THI3. |
| YDR050C | TPI1 | triosephosphate isomerase |
| YJL130C | URA2 | carbamoyl-phophate synthetase, aspartate |
| | | transcarbamylase, and glutamine |
| | | amidotransferase |
| YDL058W | USO1 | Integrin analogue gene |
| YBL047C | YBL047C | USO1 homolog (S. cerevisiae), cytoskeletal- |
| | | related transport protein, Ca++ binding |
| YBL104C | YBL104C | |
| YDR128W | YDR128W | |
| YDR279W | YDR279W | |
| YLR249W | YEF3 | EF-3 (translational elongation factor 3) |
| YHL023C | YHL023C | |
| YHR111W | YHR111W | moeB, thiF, UBA1 |
| YMR247C | YMR247C | |
| YOL138C | YOL138C | |
| YPL110C | YPL110C | |

TABLE 6
Excluded Ty protein gene identification numbers

| | | | |
|---|---|---|---|
| 7839187 | 7839173 | 6322010 | 7839201 |
| 6322347 | 7839155 | 7839171 | 6319369 |
| 7839188 | 7839156 | 7839205 | 6323688 |
| 7839162 | 6319468 | 7839159 | 6319467 |
| 7839207 | 7839160 | 7839195 | 6319485 |
| 7839180 | 7839194 | 6319486 | 6323597 |
| 6323689 | 6321110 | 7839164 | 7839199 |
| 6323695 | 6321547 | 6323601 | 7839185 |
| 6323694 | 6322486 | 6319324 | 2499832 |
| 2120056 | 141477 | 1323026 | 1323026 |
| 2499832 | 808856 | | |

TABLE 7
Hypothetical proteins identified by HMS-PCI

| ORF Name | Description | Gene |
|---|---|---|
| Q0032 | questionable ORF | |
| Q0092 | questionable ORF | |
| YAL008w | hypothetical protein | FUN14 |
| YAL017w | similarity to ser/thr protein kinases | FUN31 |
| YAL019w | similarity to helicases of the SNF2/RAD54 | FUN30 |
| | family | |
| YAL027w | hypothetical protein | |
| YAL036c | strong similarity to GTP-binding proteins | FUN11 |
| YAL049c | weak similarity to Legionella small basic | |
| | protein sbpA | |
| YAL056w | similarity to hypothetical protein YOR371c | GPE2 |
| YAR003w | similarity to human RB protein binding protein | FUN16 |
| YAR014c | similarity to hypothetical protein S. pombe | BUD14 |
| YAR044w | similarity to human oxysterol binding protein | OSH1 |
| | (OSBP) | |
| YAR060c | identical to hypothetical protein YHR212c | |
| YAR073w | strong similarity to IMP dehydrogenases | IMD1 |
| YBL004w | weak similarity to Papaya ringspot virus | |
| | polyprotein | |
| YBL029w | hypothetical protein | |
| YBL032w | weak similarity to hnRNP complex protein | |
| | homolog YBR233w | |
| YBL036c | strong similarity to C. elegans hypothetical | |
| | protein | |
| YBL044w | hypothetical protein | |
| YBL046w | weak similarity to hypothetical protein | |
| | YOR054c | |
| YBL047c | similarity to mouse eps15R protein | EDE1 |
| YBL049w | strong similarity to hypothetical protein— | |
| | human | |
| YBL051c | similarity to S. pombe Z66S68_C protein | |
| YBL055c | similarity to hypothetical S. pombe protein | |
| YBL064c | strong similarity to thiol-specific antioxidant | |
| | enzyme | |
| YBL095w | similarity to C. albicans hypothetical protein | |
| YBL104c | weak similarity to S. pombe hypothetical | |
| | protein SPAC12G12.01c | |
| YBL108w | strong similarity to subtelomeric encoded | |
| | proteins | |
| YBR014c | similarity to glutaredoxin | |
| YBR025c | strong similarity to Ylflp | |
| YBR028c | similarity to ribosomal protein kinases | |
| YBR030w | weak similarity to regulatory protein MSR1P | |
| YBR046c | similarity to zeta-crystallin | ZTA1 |
| YBR056w | similarity to glucan 1,3-beta-glucosidase | |
| YBR063c | hypothetical protein | |
| YBR066c | weak similarity to A. niger carbon catabolite | NRG2 |
| | repressor protein | |
| YBR094w | weak similarity to pig tubulin-tyrosine ligase | |
| YBR108w | weak similarity to R. norvegicus atrophin-1 | |
| | related protein | |
| YBR139w | strong similarity to carboxypeptidase | |
| YBR150c | weak similarity to transcription factors | TBS1 |
| YBR155w | weak similarity to stress-induced STI1P | CNS1 |
| YBR158w | weak similarity to TRCDSEMBL:AF176518_1 | CST13 |
| | F-box protein FBL2; human | |
| YBR175w | similarity to S. pombe beta-tranaducin | |
| YBR184w | hypothetical protein | |
| YBR187w | similarity to mouse putative tranamembrane | |
| | protein FT27 | |
| YBR203w | hypothetical protein | |
| YBR223c | hypothetical protein | |
| YBR225w | hypothetical protein | |
| YBR227c | similarity to E. coli ATP-binding protein clpX | MCX1 |
| YBR228w | similarity to hypothetical Athaliana protein | SLX1 |
| YBR239c | weak similarity to transcription factor PUT3P | |
| YBR242w | strong similarity to hypothetical protein | |
| | YGL101w | |
| YBR245c | strong similarity to D. melanogaster iswi | ISW1 |
| | protein | |
| YBR246w | similarity to TREMBL:SPCC18_15 | |
| | hypothetical protein, S. pombe | |
| YBR259w | weak similarity to ‘BH1924’, sugar transport | |
| | system; Bacillus halodurans | |
| YBR260c | similarity to C. elegans GTPase-activating | RGD1 |
| | protein | |
| YBR264c | similarity to GTP-binding proteins | YPT10 |
| YBR267w | similarity to hypothetical protein YLR3B7c | |
| YBR269c | weak similarity to ‘cpa’, phospholipase C, | |
| | Clostridium perfringens | |
| YBR270c | strong similarity to hypothetical protein | |
| | YJL058c | |
| YBR280c | similarity to hypothetical protein S. pombe | |
| YBR281c | similarity to hypothetical protein YFR044c | |
| YCL010c | strong similarity to Saccharomyces pastorianus | |
| | hypothetical protein LgYCL010c | |
| YCL039w | similarity to TUP1P general repressor of RNA | |
| | polymerase II transcription | |
| YCL048w | strong similarity to sporulation-specific protein | |
| | SPS2P | |
| YCL049c | similarity to unknown protein; S. pastorianus | |
| YCL059c | strong similarity to fission yeast rev interacting | KRR1 |
| | protein mis3 | |
| YCL061c | similarity to URK1 | MRC1 |
| YCR001w | weak similarity to chloride channel proteins | |
| YCR009c | similarity to human amphiphysin and RVS167P | RVS161 |
| YCR030c | weak similarity to S. pombe hypothetical | |
| | protein SPBC4C3.06 | |
| YCR033w | similarity to nuclear receptor co-repressor N-Cor | |
| YCR068w | similarity to starvation induced pSI-7 protein of | CVT17 |
| | C. fluvum | |
| YCR076c | weak similarity to latent transforming growth | |
| | factor beta binding protein 3′ H. sapiens | |
| YCR079w | weak similarity to A. thaliana protein | |
| | phosphatase 2C | |
| YCR087w | questionable ORF | |
| YCR099c | strong similarity to PEP1P, VTH1P and | |
| | VTH22p | |
| YCR105w | strong similarity to alcohol dehydrogenases | |
| YCR106w | similarity to transcription factor | |
| YDL001w | similarity to hypothetical protein YFR048w, | |
| | YDR282c and S. pombe hypothetical protein | |
| | SPAC12G12.14 | |
| YDL019c | similarity to SWHIP | OSH2 |
| YDL025c | similarity to probable protein kinase NPR1 | |
| YDL027c | weak similarity to hypothetical protein | |
| | Methanococcus jannaschii | |
| YDL033c | similarity to H. influenzae hypothetical protein | |
| | H10174 | |
| YDL060w | similarity to C. elegans hypothetical protein | TSR1 |
| YDL063c | weak similarity to human estrogen-responsive | |
| | finger protein | |
| YDL074c | weak similarity to spindle pole body protein | BRE1 |
| | NUF1 | |
| YDL086w | similarity to hypothetical Synechocystis protein | |
| YDL100c | similarity to E. coli arsenical pump-driving | |
| | ATPase | |
| YDL113c | similarity to hypothetical protein YDR425w | |
| YDL114w | weak similarity to Rhizobium nodulation | |
| | protein nodG | |
| YDL117w | similarity to hypothetical S. pombe protein, | CYK3 |
| | protein possibly involved in cytokinesis | |
| YDL119c | similarity to bovine Graves disease carrier | |
| | protein | |
| YDL121c | hypothetical protein | |
| YDL124w | similarity to aldose reductases | |
| YDL129w | hypothetical protein | |
| YDL156w | weak similarity to Pas7p | |
| YDL172c | questionable ORF | |
| YDL175c | weak similarity to cellular nucleic acid binding | |
| | proteins | |
| YDL193w | similarity to N. crassa hypothetical 32 kDa | |
| | protein | |
| YDL201w | strong similarity to probable methyltransferase | |
| | related protein Neurospora crassa | |
| YDL204w | similarity to hypothetical protein YDR233c | |
| YDL206w | weak similarity to transporter proteins | |
| YDL213c | weak similarity to potato small nuclear | FYV14 |
| | ribonucleoprotein U2B and human splicing | |
| | factor homolog | |
| YDL214c | strong similarity to putative protein kinase | PRR2 |
| | NPR1 | |
| YDL224c | strong similarity to WHI3 protein | WHI4 |
| YDL239c | hypothetical protein | ADY3 |
| YDL244w | strong similarity to THI5P, YJRI56c, | THI13 |
| | YNL332w and A. parasiticus, S. pombe | |
| | NMT1 protein | |
| YDL248w | strong similarity to subtelomeric encoded | COS7 |
| | proteins | |
| YDR018c | strong similarity to hypothetical protein | |
| | YBR042c | |
| YDR032c | strong similarity to S. pombe obr1 brefeldin | PST2 |
| | A resistance protein | |
| YDR036c | similarity to enoyl CoA hydratase | |
| YDR049w | similarity to C. elegans K06H7.3 protein | |
| YDR055w | strong similarity to SPS2 protein | PST1 |
| YDR063w | weak similarity to glia maturation factor beta | |
| YDR071c | similarity to G. aries arylalkylamine | |
| | N-acetyltransferase | |
| YDR091c | strong similarity to human RNase L inhibitor | RLI1 |
| | and M. jannaschii ABC transporter protein | |
| YDR093w | similarity to P. falciparum ATPase 2 | |
| YDR101c | weak similarity to proliferation-associated | |
| | protein | |
| YDR102c | hypothetical protein | |
| YDR106w | similarity to Actin proteins | ARP10 |
| YDR116c | similarity to bacterial ribosomal Li proteins | |
| YDR119w | similarity to B. subtilis tetracyclin resistance | |
| YDR124w | hypothetical protein | |
| YDR125w | weak similarity to SEC27P, YMR131c and | |
| | human retinoblastoma-binding protein | |
| YDR131c | similarity to hypothetical protein YJL149w | |
| YDR141c | strong similarity to Emericella nidulans | DOP1 |
| | developmental regulatory gene, dopey (dopA) | |
| YDR152w | weak similarity to C. elegans hypothetical | |
| | protein CET26E3 | |
| YDR161w | weak similarity to S. pombe protein of | TCI1 |
| | unknown functionSPBC16D10.01c | |
| YDR163w | weak similarity to S. pombe hypothetical | |
| | protein | |
| YDR165w | weak similarity to hypothetical C. elegans | |
| | protein | |
| YDR186c | hypothetical protein | |
| YDR196c | similarity to C. elegans hypothetical protein | |
| | T05G5.5 | |
| YDR198c | similarity to hypothetical protein S. pombe | |
| YDR200c | similarity to hypothetical protein YLR238w | |
| | similarity to A. eutrophus cation efflux system | |
| | membrane protein czcD, rat zinc transport | |
| YDR205w | protein ZnT | MSC2 |
| YDR214w | similarity to hypothetical protein YNL2S1w | |
| YDR219c | hypothetical protein | |
| YDR229w | similarity to hypothetical protein N. crassa | |
| YDR233c | similarity to hypothetical protein YDL204w | |
| YDR239c | hypothetical protein | |
| YDR247w | strong similarity to SKS1P | |
| YDR255c | weak similarity to hypothetical S. pombe | |
| | hypothetical protein SPBC29A3 | |
| YDR266c | similarity to hypothetical C. elegans protein | |
| YDR267c | weak similarity to human TAFII100 and other | |
| | WD-40 repeat containing proteins | |
| YDR274c | hypothetical protein | |
| YDR275w | weak similarity to YOR042w | |
| YDR279w | hypothetical protein | |
| YDR282c | similarity to hypothetical protein YDL001w, YFR048w | |
| | and S. pombe hypothetical protein | |
| | SPAC12G12.14 | |
| YDR287w | similarity to inositolmonophosphatases | |
| YDR295c | weak similarity to USO1P, YPR179c and fruit | PLO2 |
| | fly tropomyosin | |
| YDR303c | similarity to transcriptional regulator proteins | RSC3 |
| YDR306c | weak similarity to S. pombe hypothetical | |
| | protein SPAC6F6 | |
| YDR316w | similarity to hypothetical ubiquitin system | |
| | protein S. pombe | |
| YDR324c | weak similarity to beta transducin from S. pombe | |
| | and other WD-40 repeat containing proteins | |
| YDR326c | strong similarity to YHR080c, similarity to | |
| | YFL042c and YLR072w | |
| YDR332w | similarity to E. coli hypothetical protein | |
| | and weak similarity to RNA helicase | |
| | MSS116/YDR194c | |
| YDR339c | weak similarity to hypothetical protein | |
| | YOR004w | |
| YDR344c | hypothetical protein | |
| YDR359c | weak similarity to human trichohyalin | VID21 |
| YDR361c | similarity to hypothetical protein S. pombe | BCP1 |
| YDR365c | weak similarity to Streptococcus M protein | |
| YDR368w | strong similarity to members of the aldo/keto | |
| | reductase family YPR1 | |
| YDR372c | similarity to hypothetical S. pombe protein | |
| YDR3S0w | similarity to PDC6P, THI3P and to pyruvate | ARO10 |
| | decarboxylases | |
| YDR393w | weak similarity to rabbit trichohyalin | SHE9 |
| YDR395w | similarity to human KIAA0007 gene | |
| YDR412w | questionable ORF | |
| YDR449c | similarity to hypothetical protein S. pombe | |
| YDR452w | similarity to human sphingomyelin | PHM5 |
| | phosphodiesterase (PIR:S06957) | |
| YDR453c | strong similarity to thiol-specific antioxidant | |
| | proteins | |
| YDR459c | weak similarity to YNL326c | |
| YDR466w | similarity to ser/thr protein kinase | |
| YDR452c | hypothetical protein | |
| YDR496c | similarity to hypothetical human and | |
| | C. elegans proteins | |
| YDR506c | similarity to FET3, YFL041w and | |
| | E. floriforme diphenol oxidase | |
| YDR516c | strong similarity to glucokinase | |
| YDR527w | weak similarity to Plasmodium yoelii | |
| | rhoptry protein | |
| YEL015w | weak similarity to SPA2P | |
| YEL018w | weak similarity to RAD50P | |
| YEL023c | similarity to hypothetical protein PA2063— | |
| | Pseudomonas aeruginosa | |
| YEL025c | hypothetical protein | SRI1 |
| YEL038w | similarity to K. oxytoca enolase- | UTR4 |
| | phosphatase E-1 | |
| YEL064c | similarity to YBL089w | |
| YEL070w | strong similanty to E. coli D-mannonate | |
| | oxidoreductase | |
| YEL077c | strong similarity to subtelomeric encoded | |
| | proteins | |
| YER002w | weak similarity to chicken microfibril- | |
| | associated protein | |
| YER006w | similarity to P. polycephalum myosin-related | |
| | protein mlpA | |
| YER010c | similarity to L. pneumophila dlpA protein | |
| YER019w | weak similarity to human and mouse neutral | ISC1 |
| | sphingomyelinase | |
| YER030w | similarity to mouse nucleolin | |
| YER036c | strong similarity to members of the ABC | KRE30 |
| | transporter family | |
| YER041w | weak similarity to DNA repair protein RAD2P | YEN1 |
| | and Dsh1p | |
| YER049w | strong similarity to hypothetical S. pombe | |
| | protein YER049W | |
| YER066c-a | hypothetical protein | |
| YER066w | strong similarity to cell division control | |
| | protein CDC4P | |
| YER067w | strong similarity to hypothetical protein | |
| | YIL057c | |
| YER077c | hypothetical protein | |
| YER078c | similarity to E. coli X-Pro aminopeptidase II | |
| YER080w | hypothetical protein | |
| YER082c | similarity to M. sexta steroid regulated MNG10 | KRE31 |
| | protein | |
| YER083c | hypothetical protein | |
| YER084w | questionable ORF | |
| YER087w | similarity to E. coli prolyl-tRNA synthetase | |
| YER093c | weak similarity to S. epidermidis PepB protein | |
| YER124c | weak similarity to Dictyostelium WD40 repeat | |
| | protein 2 | |
| YER126c | weak similarity to E. coli colicin N | KRE32 |
| YER130c | similarity to MSN2P and weak similarity to | |
| | MSN4P | |
| YER140w | similarity to PIR:T39406 hypothetical protein | |
| | S. pombe | |
| YER158c | weak similarity to AFR1P | |
| YER166w | similarity to ATPase P. falciparum ATPase 2 | |
| YER182w | similarity to hypothetical protein | |
| | SPAC3A12.08—S. pombe | |
| YER184c | similarity to multidrug resistance proteins | |
| | PDR3P and PDR1P | |
| YER185w | strong similarity to Rtm1p | |
| YFL006w | similarity to hypothetical protein | |
| | TRCDSEMBL:AB024034_15 A. thaliana | |
| YFL007w | weak similarity to Mms19p | BLM3 |
| YFL013c | weak similarity to Dictyostelium protein kinase | IES1 |
| YFL024c | weak similarity to YMR164c and GAL11P | EPL1 |
| YFL027c | weak similarity to P. falciparum Pfmdr2 | |
| | protein | |
| YFL030w | similarity to several transaminases | |
| YFL034w | similarity to hypothetical S. pombe protein | |
| | and to C. elegans F35D11 protein | |
| YFL042c | similarity to hypothetical protein YLR072w | |
| YFL054c | similarity to channel proteins | |
| YFR001w | weak similarity to rabbit triadin SPP41P | L0C1 |
| YFR003c | strong similarity to hypothetical protein | |
| | SPAC6B12.13—S. pombe | |
| YFR008w | weak similarity to human centromere protein E | |
| YFR016c | similarity to mammalian neurofilament proteins | |
| | and to Dictyostelium protein kinase | |
| YFR017c | hypothetical protein | |
| YFR021w | similarity to hypothetical protein YPL100w | NMR1 |
| YFR024c-a | similarity to Acanthamoeba myosin heavy | |
| | chain IC and weak similarity to other myosin | |
| | class I heavy c | |
| YFR039c | similarity to hypothetical protein YGL228w | |
| YFR044c | similarity to hypothetical protein YBR281c | |
| YGL004c | weak similarity to TUP1P | |
| YGL020c | weak similarity to | |
| | TRCDSEMBL:SPBC543_10 putative | |
| | coiled-coil protein S. pombe | |
| YGL037c | similarity to PIR:B70386 pyrazinamidase/ | PNC1 |
| | nicotinamidase—Aquifex aeolicus | |
| YGL057c | hypothetical protein | |
| YGL059w | similarity to rat branched-chain alpha-ketoacid | |
| | dehydrogenase kinase | |
| YGL060w | strong similarity to hypothetical protein | |
| | YBR216c | |
| YGL068w | strong similarity to Cricetus mitochrondial | |
| | ribosomal L12 protein | |
| YGL081w | hypothetical protein | |
| YGL083w | weak similarity to bovine rhodopsin kinase and | SCY1 |
| | to YGR052w | |
| YGL096w | similarity to copper homeostasis protein | TOS8 |
| | CUP9P | |
| YGL099w | similarity to putative human GTP-binding | KRE35 |
| | protein MMR1 | |
| YGL101w | strong similarity to hypothetical protein | |
| | YBR242w | |
| YGL104c | similarity to glucose transport proteins | |
| YGL110c | similarity to hypothetical protein | |
| | SPCC1906.02c S. pombe | |
| YGL111w | weak similarity to hypothetical protein | |
| | S. pombe | |
| YGL113w | weak similarity to YOR165w | SLD3 |
| YGL117w | hypothetical protein | |
| YGL121c | hypothetical protein | |
| YGL129c | similarity to S. pombe pl hypothetical protein | RSM23 |
| | SPBC29A3.15C—putative mitochondrial | |
| | function | |
| YGL131c | weak similarity to S. pombe hypothetical | |
| | protein C3H1.12C | |
| YGL140c | weak similarity to Lactobacillus putative histidine | |
| | protein kinase SppK | |
| YGL146c | hypothetical protein | |
| YGL150c | similarity to SNF2P and human SNF2alpha | INO80 |
| YGL174w | weak similarity to C. elegans hypothetical | BUD13 |
| | protein R08D7.1 | |
| YGL179c | strong similarity to PAK1P, ELM1P and | TOS3 |
| | KIN82P | |
| YGL184c | strong similarity to Emericella nidulans and | STR3 |
| | similarity to other cystathionine beta-lyase and | |
| | CYS3P | |
| YGL220w | weak similarity to V. alginolyticus bolA protein | |
| YGL222c | weak similarity to EDC2 | EDC1 |
| YGL227w | weak similarity to human RANBPM | VID30 |
| | NP_005484.1 | |
| YGL228w | similarity to hypothetical protein YFR039c | SHE10 |
| YGL245w | strong similanty to glutamine—tRNA ligase | |
| YGL246c | weak similarity to C. elegans dom-3 protein | RAI1 |
| YGR002c | similarity to hypothetical S. pombe protein | |
| YGR004w | strong similarity to hypothetical protein | |
| | YLR324w | |
| YGR016w | weak similarity to M. jannaschii hypothetical | |
| | protein MJ1317 | |
| YGR017w | weak similarity to | |
| | TRCDSEMBL:AC006418_11 A. thaliana | |
| YGR021w | similarity to M. leprae yfcA protein | |
| YGR033c | weak similarity to | |
| | TRCDSEMBLNEW:AP002861_10 | |
| | Oryza sativa | |
| YGR042w | weak similarity to TRCDSEMBL:CH20111_1 | |
| | Troponin-I; Clupea harengus | |
| YGR043c | strong similarity to transaldolase | |
| YGR052w | similarity to ser/thr protein kinases | |
| YGR054w | similarity to C. elegans E04D5.1 protein | |
| YGR066c | similarity to hypothetical protein YBR105c | |
| YGR067c | weak similarity to transcription factors | |
| YGR073e | questionable ORF | |
| YGR077c | similarity to Hansenula polymorpha PER1 | PEX8 |
| | protein and weak similanty to Pichia pastoris | |
| | PER3 protein | |
| YGR086c | strong similarity to hypothetical protein | |
| | YPL004c | |
| YGR090w | similarity to PIR:T40678 hypothetical protein | |
| | SPBC776.08c S. pombe | |
| YGR103w | similarity to zebrafish essential for embryonic | |
| | development gene pescadillo | |
| YGR110w | weak similarity to YLR099c and YDR125c | |
| YGR111w | weak similarity to mosquito carboxylesterase | |
| YGR128c | hypothetical protein | |
| YGR130c | weak similarity to myosin heavy chain proteins | |
| YGR134w | hypothetical protein | CAF130 |
| YGR136w | weak similarity to chicken growth factor | |
| | receptor-binding protein GRB2 homolog | |
| YGR145w | similarity to C. elegans hypothetical | |
| | protein | |
| YGR150c | similarity to PIR:T39838 hypothetical protein | |
| | SPBC19G7.07c S. pombe | |
| YGR154c | strong similarity to hypothetical proteins | |
| | YKR076w and YMR251w | |
| YGR161c | hypothetical protein | |
| YGR165w | similarity to PIR:T39444 hypothetical protein | |
| | SPBC14C8.16c S. pombe | |
| YGR169c | similarity to RIB2P | |
| YGR173w | strong similarity to human GTP-binding protein | |
| YGR187c | weak similarity to human HMG1P and HMG2P | HGH1 |
| YGR196c | weak similarity to Tetrahymena acidic | FYV8 |
| | repetitive protein ARP1 | |
| YGR198w | weak similarity to PIR:T38996 hypothetical | |
| | protein SPAC637.04 S. pombe | |
| YGR200c | weak similarity to rape guanine nucleotide | ELP2 |
| | regulatory protein | |
| YGR205w | similarity to S. pombe hypothetical protein | |
| | D89234 | |
| YGR210c | similarity to M. jannaschii GTP-binding protein | |
| | and to M. caprtcolum hypothetical protein | |
| | SGC3 | |
| YGR223c | weak similarity to hypothetical protein | |
| | YFR021w | |
| YGR235c | hypothetical protein | |
| YGR243w | strong similarity to hypothetical protein | |
| | YHR162w | |
| YGR250c | weak similarity to human cleavage stimulation | |
| | factor 64K chain | |
| YGR262c | weak similarity to protein kinases and M. | BUD32 |
| | jannaschii O-sialoglycoprotein endopeptidase | |
| | homolog | |
| YGR263c | weak similarity to E. coli lipase like enzyme | |
| YGR266w | hypothetical protein | |
| YGR271w | strong similarity to S. pombe RNA helicase | SLH1 |
| YGR278w | similarity to C. elegans LET-858 | |
| YGR279c | similarity to glucanase | SCW4 |
| YGR280c | weak similarity to CBF5P | |
| YGR296w | strong similarity to YPL283c; YNL339c and | YRF1-3 |
| | other Y encoded proteins | |
| YHL010c | similarity to C. elegans hypothetical protein, | |
| | homolog to human breast cancer-associated | |
| | protein BRAP | |
| YHL013c | similarity to C. elegans hypothetical protein | |
| | F21D5.2 | |
| YHL014c | similarity to E. coli GTP-binding protein | YLF2 |
| YHL017w | strong similarity to PTM1P | |
| YHL023c | weak similarity to TRCDSEMBL:SPBC543_4 | |
| | hypothetical protein S. pombe | |
| YHL026c | similarity to PIR:T41446 conserved | |
| | hypothetical protein SPCC594.02c S. pombe | |
| YHL035c | similarity to multidrug resistance proteins | |
| YHL039w | weak similarity to YPL208w | |
| YHR001w | similarity to KES1P | OSH7 |
| YHR002w | similarity to bovine mitochondrial carrier | |
| | protein/Grave's disease carrier protein | |
| YHR009c | similarity to S. pombe hypothetical protein | |
| YHR011w | strong similarity to seryl-tRNA synthetases | DIA4 |
| YHR016c | strong similarity to hypothetical protein | YSC84 |
| | YFR024c-a | |
| YHR020w | strong similarity to human glutamyl-prolyl- | |
| | tRNA synthetase and fruit fly multifunctional | |
| | aminoacyl-t | |
| YHR022c | weak similarity to ras-related protein | |
| YHR033w | strong similarity to glutamate 5-kinase | |
| YHR035w | weak similarity to human SEC23 protein | |
| YHR040w | weak similarity to HIT1P | |
| YHR045w | hypothetical protein | |
| YHR046c | similarity to inositolmonophosphatases | |
| YHR052w | weak similarity to P. yoelii rhoptry protein | |
| YHR056c | strong similarity to YHR054c | RSC30 |
| YHR059w | weak similarity to Ustilago hordei B east | FYV4 |
| | mating protein 2 | |
| YHR063c | weak similarity to translational activator CBS2 | PAN5 |
| YHR070w | strong similarity to N. crassa met-10+ protein | TRM5 |
| YHR073w | similarity to OSH1P, YDL019c and mammalian | OSH3 |
| | oxysterol-binding protei | |
| YHR074w | weak similarity to B. subtilis spore outgrowth | QNS1 |
| | factor B | |
| YHR076w | weak similarity to C. elegans hypothetical | |
| | protein CEW09D10 | |
| YHR080c | similarity to hypothetical protein YDR326c, | |
| | YFL042c and YLR072w | |
| YHR087w | weak similarity to PIR:T50363 hypothetical | |
| | protein SPBC21C3.19 S. pombe | |
| YHR088w | similarity to hypothetical protein YNL07Sw | RPF1 |
| YHR098c | similarity to human hypothetical protein | SFB3 |
| YHR100c | strong similarity to PIR:T48794 hypothetical | |
| | protein Neurospora crassa | |
| YHR105w | weak similarity to MVP1P | |
| YHR111w | similarity to molybdopterin biosynthesis | |
| | proteins | |
| YHR112c | similarity to cystathionine gamma-synthases | |
| YHR113w | similarity to vacuolar aminopeptidase Ape1p | |
| YHR114w | similarity to S. pombe hypothetical protein | BZZ1 |
| | and human protein-tyrosine kinase fer | |
| YHR115c | strong similarity to hypothetical protein | |
| | YNL116w | |
| YHR122w | similarity to hypothetical C. elegans protein | |
| | F45G2.a | |
| YHR149c | similarity to hypothetical protein YGR221c | |
| YHR169w | strong similarityto DRS1P and other probable | DBP8 |
| | ATP-dependent RNA helicases | |
| YHR177w | weak similarity to S. pombe PAC2 protein | |
| YHR182w | weak similarity to PIR:S58162 probable Rho | |
| | GTPase protein S. pombe | |
| YHR186c | similarity to C. elegans hypothetical protein | |
| | C10C5.6 | |
| YHR188c | similarity to hypothetical C. elegans proteins | GPI16 |
| | F17c11.7 | |
| YHR196w | weak similarity to YDR398w | |
| YHR197w | weak similarity to PIR:T22172 hypothetical | |
| | protein F44E5.2 C. elegans | |
| YHR199c | strong similarity to hypothetical protein | |
| | YHR198c | |
| YHR209w | similarity to hypothetical protein YER175c | |
| YHR214w-a | strong similarity to hypothetical protein | |
| | YAR068w | |
| YIL005w | similarity to protein disulfide isomerases | |
| YIL007c | similarity to C. elegans hypothetical protein | |
| YIL017c | similarity to S. poinbe SPAC26H5.04 protein | VID28 |
| | of unknown function | |
| YIL028w | hypothetical protein | |
| YIL037c | weak similarity to C. elegans F26G1.6 protease | PRM2 |
| YIL055c | hypothetical protein | |
| YIL077c | hypothetical protein | |
| YIL079c | strong similarity to hypothetical protein | |
| | YDL175c | |
| YIL091c | weak similarity to SPT5P | |
| YIL093c | weak similarity to S. pombe hypothetical | RSM25 |
| | protein SPBC16A3 | |
| YIL097w | weak similanty to erythroblast macrophage | FYV10 |
| | protein EMP Mus musculus | |
| YIL104c | similarity to hypothetical S. pombe protein | |
| YIL105c | weak similarity to probable transcription factor | |
| | ASK10P | |
| YIL108w | similarity to hypothetical S. pombe protein | |
| YIL112w | similarity to ankyrin and coiled-coil proteins | |
| YIL113w | strong similarity to dual-specificity phosphatase | |
| | MSG5P | |
| YIL117c | similarity to hypothetical protein YNL058c | PRM5 |
| YIL120w | similarity to antibiotic resistance proteins | QDR1 |
| YIL137c | similarity to M. musculus aminopeptidase | |
| YIL164c | strong similarity to nitrilases, putative | NIT1 |
| | pseudogene | |
| YIR00Lc | similarity to D. melanogaster RNA binding | SGN1 |
| | protein | |
| YIR003w | weak similarity to mammalian neurofilament | |
| | triplet H proteins | |
| YIR005w | similarity to RNA-binding proteins | IST3 |
| YIR007w | hypothetical protein | |
| YIR035c | similarity to human corticosteroid | |
| | 11-beta-dehydrogenase | |
| YJL019w | weak similarity to hypothetical protein | |
| | C. elegans | |
| YJL020c | similarity to S. pombe hypothetical protein | BBC1 |
| | SPAC23A1.16 | |
| YJL038c | strong similarity to hypothetical protein | |
| | YJL037w | |
| YJL045w | strong similarity to succinate dehydrogenase | |
| | flavoprotein | |
| YJL047c | weak similarity to CDC53P | RTT101 |
| YJL051w | hypothetical protein | |
| YJL066c | hypothetical protein | MPMt |
| YJL068c | strong similarity to human esterase D | |
| YJL069c | similarity to C. elegans hypothetical protein | |
| YJL070c | similarity to AMP deaminases | |
| YJL073w | similarity to heat shock proteins | JEM1 |
| YJL082w | strong similarity to hypothetical protein | IML2 |
| | YKR018c | |
| YJL084c | similarity to hypothetical protein YKR021w | |
| YJL105w | similarity to hypothetical protein YKR029c | |
| YJL107c | similarity to hypothetical S. pombe protein | |
| YJL109c | weak similarity to ATPase DRS2P | |
| YJL122w | weak similarity to dog-fish transition protein 52 | |
| YJL132w | weak similarity to human phospholipase D | |
| YJL149w | similarity to hypothetical protein YDR131c | |
| YJL181w | similarity to hypothetical protein YJR030c | |
| YJL204c | weak similarity to TOR2P | |
| YJL207c | weak similarity to rat omega-conotoxin- | |
| | sensitive calcium channel alpha-1 subunit rbB-I | |
| YJL211c | questionable ORF | |
| YJR011c | hypothetical protein | |
| YJR024c | weak similarity to C. elegans Z49131_E | |
| | ZC373.5 protein | |
| YJR041c | weak similarity to hypothetical protein | |
| | SPAC2G11.02 S. pombe | |
| YJR054w | similarity to hypothetical protein YML047c | |
| YJR061w | similarity to MNN4P | |
| YJR070c | similarity to C. elegans hypothetical protein | |
| | C14A4.1 | |
| YJR072c | strong similarity to C. elegans hypothetical | |
| | protein and similarity to YLR243w | |
| YJR078w | similarity to mammalian indoleamine | |
| | 2,3-dioxygenase | |
| YJR080c | hypothetical protein | |
| YJR087w | questionable ORF | |
| YJR100c | weak similarity to BUD3P | |
| YJR101w | weak similarity to superoxide dismutases | RSM26 |
| YJR105w | strong similarity to human adenosine kinase | ADO1 |
| YJR110w | similarity to human myotubularmn | |
| YJR119c | similarity to human retinoblastoma binding | |
| | protein 2 | |
| YJR126c | similarity to human prostate-specific membrane | |
| | antigen and transferrin receptor protein | |
| YJR129c | weak similarity to hypothetical protein | |
| | YNL024c | |
| YJR134c | similarity to paramyosin, myosin | SGM1 |
| YJR138w | similarity to C. elegans hypothetical protein | IML1 |
| | T0BA11.1 | |
| YJR141w | weak similarity to hypothetical protein | |
| | SPBC1734.10c S. pombe | |
| YJR149w | similarity to 2-nitropropane dioxygenase | |
| YJR151c | similarity to mucus proteins, YKL224c, Sta1p | DAN4 |
| YKL010c | similarity to rat ubiquitin ligase Nedd4 | UFD4 |
| YKL014c | similarity to hypothetical protein | |
| | SPCC14G10.02 S. pombe | |
| YKL018w | similarity to C. elegans hypothetical protein | |
| YKL034w | weak similarity to YOL013c | |
| YKL036c | questionable ORF | |
| YKL047w | hypothetical protein | |
| YKL054c | similarity to glutenin, high molecular weight | VID31 |
| | chain proteins and SNF5P | |
| YKL056c | strong similarity to human 1gB-dependent | |
| | histamine-releasing factor | |
| YKL075c | hypothetical protein | |
| YKL082c | weak similarity to C. elegans hypothetical | |
| | protein | |
| YKL088w | similarity to C. tropicalis hal3 protein, to | |
| | C-term of SIS2P and to hypothetical protein | |
| | YOR054c | |
| YKL095w | similarity to C. elegans hypothetical proteins | YJU2 |
| YKL099c | similarity to C. elegans hypothetical proteins | |
| | C18G6.06 and C16C10.2 | |
| YKL105c | similarity to YMR086w | |
| YKL116c | similarity to rat SNF1, C. elegans unc-51, | PRR1 |
| | DUN1P and other protein seine kinases | |
| YKL120w | similarity to mitochondrial uncoupling proteins | OAC1 |
| | (MCF) | |
| YKL121w | strong similarity to YMR102c | |
| YKL133c | similarity to hypothetical protein YMR115w | |
| YKL155c | similarity to S. pombe SPAC1420.04c putative | RSM22 |
| | cytochrome c oxidase assembly protein | |
| YKL161c | strong similarity to ser/thr-specific protein | (MLP1) |
| | kinase SLT2P | |
| YKL179c | similarity to NUF1P | |
| YKL189w | similarity to mouse hypothetical calcium- | HYM1 |
| | binding protein and D. melanogaster | |
| | Mo25 gene | |
| YKL195w | similarity to rabbit histidine-rich calcium- | |
| | binding protein | |
| YKL206c | hypothetical protein | |
| YKL214c | weak similarity to mouse transcriptional | |
| | coactivator ALY | |
| YKL215c | similarity to P. aeruginosa hyuA and hyuB | |
| YKL218c | strong similarity to E. coli and H. influenzae | SRY1 |
| | threonine dehydratases | |
| YKL222c | weak similarity to transcription factors, | |
| | similarity to finger proteins YOR162c, | |
| | YOR172w and YLR266c | |
| YKR005c | hypothetical protein | |
| YKR007w | weak similarity to Streptococcus protein M5 | |
| | precursor | |
| YKR017c | similarity to human hypothetical KIAA0161 | |
| | protein | |
| YKR018c | strong similarity to hypothetical protein | |
| | YJL082w | |
| YKR020w | hypothetical protein | |
| YKR029c | similarity to YJL105w and Lentinula MFBA | |
| | protein | |
| YKR038c | similarity to QR17P | |
| YKR046c | hypothetical protein | |
| YKR051w | similarity to C. elegans hypothetical protein | |
| YKR060w | similarity to hypothetical protein S. pombe | |
| YKR064w | weak similarity to transcription factors | |
| YKR065c | similarity to hypothetical protein S. pombe | |
| YKR067w | strong similarity to SCT1P | |
| YKR079c | similarity to S. pombe hypothetical protein | |
| | SPAC1D4.10 | |
| YKR081c | strong similarity to hypothetical protein | |
| | S. pombe | |
| YKR090w | similarity to chicken Lim protein kinase and | |
| | Islet proteins | |
| YKR096w | similarity to mitochondrial aldehyde | |
| | dehydrogenase Ald1p | |
| YLL013c | similarity to Drosophila pumilio protein | |
| YLL015w | similarity to YCF1P, YOR1P, rst organic anion | BPT1 |
| | transporter | |
| YLL029w | similarity to M. jannaschii X-Pro dipeptidase | |
| | and S. pombe hypothetical protein | |
| YLL034c | similarity to mammalian valosin | |
| YLL038c | weak similarity to YJR125c and YDL161w | ENT4 |
| YLL054c | similarity to transcription factor PIP2P | |
| YLL063e | strong similarity to Gibberella zeae | AYT1 |
| | trichothecene 3-O-acetyltransferase | |
| YLR002c | similarity to hypothetical C. elegans protein | |
| YLR009w | similarity to ribosomal protein L24.e.B | |
| YLR015w | weak similarity to S. pombe hypothetical | BRE2 |
| | protein SPBC13G1 | |
| YLR016c | weak similarity to | |
| | TRCDSEMBLNEW:AK022615_1 unnamed | |
| | ORF; Homo sapiens | |
| YLR024c | similarity to ubiquitin—protein ligase UBR1P | UBR2 |
| YLR035c | similarity to human mutL protein homolog, | MLH2 |
| | mouse PMS2, MLH1P and PMS1P | |
| YLR062e | questionable ORF | BUD28 |
| YLR063w | hypothetical protein | |
| YLR070c | strong similarity to sugar dehydrogenases | |
| YLR074c | weak similarity to human zinc finger protein | BUD20 |
| YLR080w | strong similarity to EMP47P | |
| YLR087c | weak similarity to hypothetical protein | CSF1 |
| | D. melanogaster | |
| YLR097c | weak similarity to H. sapiens F-box protein | |
| YLR106c | similarity to Kaposi's sarcoma-associated | |
| | herpes-like virus ORF73 homolog gene | |
| YLR117c | strong similarity to Drosophila putative cell | CLF1 |
| | cycle control protein cm | |
| YLR122c | hypothetical protein | |
| YLR152c | similarity to YOR3165w and YNL095c | |
| YLR154c | hypothetical protein | |
| YLR177w | similarity to suppressor protein Psp5p | |
| YLR179c | similarity to TFS1P | |
| YLR183c | similarity to YDR501w | TOS4 |
| YLR186w | strong similarity to S. pombe hypothetical | EMG1 |
| | protein C18C36.07C | |
| YLR187w | similarity to hypothetical protein YNL278w | |
| YLR193c | similarity to G. gallus pxt9 and MSF1P | |
| YLR196w | similarity to human IEF SSP 9502 protein | PWP1 |
| YLR199c | hypothetical protein | |
| YLR205c | hypothetical protein | |
| YLR211e | hypothetical protein | |
| YLR215c | strong similarity to rat cell cycle progression | CDC123 |
| | related D123 protein | |
| YLR219w | hypothetical protein | MSC3 |
| YLR222c | similarity to DIP2P | CST29 |
| YLR231c | strong similarity to rat kynureninase | |
| YLR238w | similarity to YDR200c | |
| YLR241w | similarity to hypothetical S. pombe | |
| | protein SPAC2G11.09 | |
| YLR243w | strong similarity to YOR262w | |
| YLR247c | similarity to S. pombe rad8 protein and | |
| | RDH54P | |
| YLR266c | weak similarity to transcription factors | |
| YLR267w | hypothetical protein | BOP2 |
| YLR270w | strong similarity to YOR173w | |
| YLR271w | weak similarity to hypothetical protein | |
| | T04H1.5 C. elegans | |
| YLR276c | similarity to YDL031w, MAK5P and RNA | DBP9 |
| | helicases | |
| YLR282c | questionable ORF | |
| YLR287c | weak similarity to S. pombe hypothetical | |
| | protein SPAC22E12 | |
| YLR289w | strong similarity to E. coli elongation | GUF1 |
| | factor-type GTP-hinding protein lepa | |
| YLR320w | hypothetical protein | |
| YLR323c | weak similarity to N. crassa uvs2 protein | |
| YLR324w | strong similarity to YGR004w | |
| YLR326w | hypothetical protein | |
| YLR328w | strong similarity to YGR010w | |
| YLR331c | questionable ORF | |
| YLR349w | questionable ORF | |
| YLR352w | hypothetical protein | |
| YLR368w | weak similarity to Mus musculus F-box | |
| | protein FBA | |
| YLR373c | similarity to hypothetical protein Ygr071cp | VID22 |
| YLR381w | hypothetical protein | |
| YLR386w | similarity to hypothetical S. pombe protein | |
| YLR392c | hypothetical protein | |
| YLR397c | strong similarity to CDC48 | AFG2 |
| YLR400w | hypothetical protein | |
| YLR401c | similarity to A. brasilense nifR3 protein | |
| YLR405w | similarity to A. brasilense nifR3 protein | |
| YLR409c | strong similarity to S. pombe beta-transducin | |
| YLR410w | strong similarity to S. pombe protein ASP1P | VIP1 |
| YLR413w | strong similarity to YKL187c | |
| YLR415c | questionable ORF | |
| YLR419w | similarity to helicases | |
| YLR421c | weak similarity to human 42K membrane | RPN13 |
| | glycoprotein | |
| YLR422w | similarity to human DOCK180 protein | |
| YLR424w | weak similarity to STU1P | |
| YLR425w | similarity to GDP-GTP exchange factors | TUS1 |
| YLR426w | weak similarity to 3-oxoacyl-[acyl-carrier- | |
| | protein] reductase from E. coli | |
| YLR427w | weak similarity to human transcription | |
| | regulator Staf-5 | |
| YLR432w | strong similarity to IMP dehydrogenases, Pur5p | IMD3 |
| | and YML056c | |
| YLR454w | similarity to YPR117w | |
| YLR460c | similarity to C. carbonum toxD protein | |
| YML002w | hypothetical protein | |
| YML005w | similarity to hypothetical S. pombe protein | |
| YML006c | hypothetical protein | GIS4 |
| YML020w | hypothetical protein | |
| YML023c | weak similarity to NMD2P | |
| YML029w | hypothetical protein | |
| YML034w | similarity to YDR458c | SRC1 |
| YML036w | weak similarity to C. elegans hypothetical | |
| | protein CELW03F8 | |
| YML056c | strong similarity to IMP dehydrogenases | IMD4 |
| YML059c | similarity to C. elegans ZK370.4 protein | |
| YML068w | similarity to C. elegans hypothetical protein | |
| YML072c | similarity to YOR3141c and YNL087w | |
| YML076c | weak similarity to transcription factor | |
| YML081w | strong similarity to ZMS1 protein | |
| YML093w | similarity to P. falciparum liver stage | |
| | antigen LSA-1 | |
| YML111w | strong similarity to ubiquitination protein | BUL2 |
| | BUL1P | |
| YML117w | similarity to YPL184c | |
| YML128c | weak similarity to S. pombe SPBC365.12c | MSC1 |
| | protein of unknown function | |
| YMR015w | similarity to tetratricopeptide-repeat protein | |
| | PAS10 | |
| YMR019w | weak similarity to YIL130w, PUT3P and | STB4 |
| | other transcription factors | |
| YMR029c | weak similarity to human nuclear autoantigen | |
| YMR030w | hypothetical protein | |
| YMR049c | weak similarity to A. thaliana PRL1 protein | ERB1 |
| YMR066w | hypothetical protein | SOV1 |
| YMR068w | weak similarity to mouse transcription factor | |
| | NF-kappaB | |
| YMR074c | strong similarity to hypothetical S. pombe | |
| | protein | |
| YMR086w | similarity to YKLL05c | |
| YMR093w | weak similarity to PWP2P | |
| YMR099c | similarity to P. ciliare possible | |
| | apospory-associated protein | |
| YMR102c | strong similarity to YKL121w | |
| YMR135c | weak similarity to conserved hypothetical | |
| | protein S. pombe | |
| YMR144w | weak similarity to MLP1P | |
| YMR155w | weak similarity to E. coli hypothetical | |
| | protein f402 | |
| YMR172w | similarity to MSN1 protein | HOT1 |
| YMR196w | strong similarity to hypothetical protein | |
| | Neurospora crassa | |
| YMR206w | weak similarity to hypothetical protein | |
| | YNR014w | |
| YMR207c | strong similarity to acetyl-CoA carboxylase | HFA1 |
| YMR209c | similarity to conserved hypothetical protein | |
| | S. pombe | |
| YMR223w | similarity to mouse deubiquitinating enzyme | UBP8 |
| | and UBP13P, UBP9, DOA4P | |
| YMR226c | similarity to ketoreductases | |
| YMR233w | strong similarity to YOR295w | |
| YMR247c | similarity to hypothetical protein S. pombe | |
| YMR250w | similarity to glutamate decarboxylases | GAD1 |
| YMR251w | strong similarity to YKR076w and YGR154c | |
| YMR259c | similarity to hypothetical protein S. pombe | |
| YMR265c | weak similarity to hypothetical protein | |
| | S. pombe | |
| YMR266w | similarity to A. thaliana hyp1 protein | RSN1 |
| YMR278w | similarity to phosphomannomutases | |
| YMR285c | similarity to CCR4P | NGL2 |
| YMR289w | weak similarity to para-aminobenzoate synthase | |
| | component I (EC 4.1.3.-) Campylobacter jejuni | |
| YMR291w | similarity to ser/thr protein kinase | |
| YMR304w | similarity to human ubiquitin-specific protease | UBP15 |
| YMR306w | similarity to 1,3-beta-glucan synthases | FKS3 |
| YMR315w | similarity to hypothetical S. pombe protein | |
| YMR316w | similarity to YOR385w and YNL165w | DIA1 |
| YMR318c | strong similarity to alcohol-dehydrogenase | |
| YMR323w | strong similarity to phosphopyruvate hydratases | |
| YNL102c | strong similarity to mammalian ribosomal L7 | RLP7 |
| | proteins | |
| YNL004w | strong similarity to GBP2P | HRB1 |
| YNL008c | similarity to YMR119w | |
| YNL023e | similarity to D. melanogaster shuttle craft | FAP1 |
| | protein | |
| YNL032w | similarity to YNL099c, YNL056w and | SIW14 |
| | YDR067c | |
| YNL035c | similarity to hypothetical protein S. pombe | |
| YNL040w | weak similarity to M. genitalium alanine— | |
| | tRNA ligase | |
| YNL045w | strong similarity to human leukotriene-A4 | |
| | hydrolase | |
| YNL047c | similarity to probable transcription factor | |
| | ASK10P and hypothetical protein YPR115w, and | |
| | strong simi | |
| YNL051w | weak similarity to hypothetical protein | |
| | Drosophila melanogaster | |
| YNL056w | similarity to YNL032w and YNL099c | |
| YNL063w | weak similarity to Mycoplasma | |
| | protoporphyrinogen oxidase | |
| YNL078w | hypothetical protein | |
| YNL083w | weak similarity to rabbit peroxisomal | |
| | Ca-dependent solute carrier | |
| YNL091w | similarity to chicken h-caldesmon, USO1P | |
| | and YKL201c | |
| YNL094w | similarity to S. pombe hypothetical protein | |
| YNL096c | strong similarity to ribosomal protein S7 | RPS7B |
| YNL099c | similarity to YNL032w, YNL056w and | |
| | YDR067c | |
| YNL107w | similarity to human AF-9 protein | YAF9 |
| YNL108c | strong similarity to YOR110w | |
| YNL109w | weak similarity to cytochrome-c oxidase | |
| YNL110c | weak similarity to fruit fly RNA-binding | |
| | protein | |
| YNL116w | weak similarity to RING zinc finger protein | |
| | from Gallus gallus | |
| YNL123w | weak similarity to C. jejuni acme protease | |
| YNL124w | similarity to hypothetical S. pombe protein | |
| YNL127w | similarity to C. elegans hypothetical protein | |
| YNL128w | weak similarity to tensin and to the mammalian | TEP1 |
| | tumor suppressor gene product | |
| | MMAC1/PTEN/TEP1 | |
| YNL132w | similarity to A. ambisexualis anthendiol | KRE33 |
| | steroid receptor | |
| YNL134c | similarity to C. carbonum toxD gene | |
| YNL144c | similarity to YHR131c | |
| YNL157w | weak similarity to S. pombe hypothetical | |
| | protein SPAC10F6 | |
| YNL161w | strong similarity to U. maydis Ukc1p protein | CBK1 |
| | kinase | |
| YNL166c | similarity to S. pombe SPBC1711.05 | BNI5 |
| | serine-rich repeat protein of unknown function | |
| YNL175c | similarity to S. pombe Rnp24p, NSR1P and | NOP13 |
| | human splicing factor | |
| YNL180c | similarity to S. pombe CDC42P and other | RHO5 |
| | GTP-hinding proteins | |
| YNL181w | similarity to hypothetical S. pombe protein | |
| YNL182c | weak similarity to S. pombe hypothetical | |
| | protein | |
| YNL201c | weak similarity to pleiotropic drug resistance | |
| | control protein PDR6 | |
| YNL207w | similarity to M. jannaschii hypothetical protein | |
| | MJ1073 | |
| YNL208w | weak similarity to Colletotrichum | |
| | gloeosporioides nitrogen starvation-induced | |
| | glutamine rich protein | |
| YNL213c | similarity to hypothetical protein Neurospora | |
| | crassa | |
| YNL217w | weak similarity to E. coli bis(5′-nucleosyl)- | |
| | tetraphosphatase | |
| YNL227c | similarity to dnaJ-like proteins | |
| YNL230c | weak similarity to mammalian transcription | ELA1 |
| | elongation factor elongin A | |
| YNL253w | similarity to hypothetical protein C. elegans | |
| YNL255c | strong similarity to nucleic acid-binding | GIS2 |
| | proteins, similarity to Tetrahymena thermophila | |
| | cnjB prote | |
| YNL260c | weak similarity to hypothetical protein | |
| | S. pombe | |
| YNL275w | similarity to human band 3 anion transport | |
| | protein | |
| YNL278w | similarity to YLR187w | CAF120 |
| YNL279w | similarity to S. pombe coiled-coil protein of | PRM1 |
| | unknown function | |
| YNL281w | strong similarity to YDR214w | HCH1 |
| YNL294c | similarity to TRCDSEMBL:AF152926_1 | |
| | pa1H Emericella nidulans | |
| YNL308c | similarity to S. pombe and C. elegans | KRI1 |
| | hypothetical proteins | |
| YNL311c | hypothetical protein | |
| YNL313c | similarity to C. elegans hypothetical protein | |
| YNL320w | strong similarity to S. pombe Bem46 protein | |
| YNL321w | weak similarity to VCX1P | |
| YNL323w | similarity to Ycx1p | LEM3 |
| YNL334c | strong similarity to hypothetical proteins | SNO2 |
| | YFL060c and YMR095c | |
| YNR018w | similarity to TRCDSEMBL:SPAC1565_1 | |
| | hypothetical protein S. pombe | |
| YNR021w | weak similarity to hypothetical protein | |
| | S. pombe | |
| YNR039c | weak similarity to Anopheles mitochondrial | ZRG17 |
| | NADH dehydrogenase subunit 2 | |
| YNR047w | similarity to ser/thr protein kinases | |
| YNR053c | strong similarity to human breast tumor | |
| | associated autoantigen | |
| YNR054e | similarity to C. elegans hypothetical protein | |
| | CEESL47F | |
| YNR065c | strong similarity to YJL222w, YIL173w and | |
| | PEP1P | |
| YNR066c | strong similarity to PEP1P | |
| Y0L010w | similarity to human RNA 3-terminal phosphate | RCL1 |
| | cyclase | |
| Y0L027c | similarity to YPR125w | |
| Y0L029c | hypothetical protein | |
| Y0L034w | similarity to S. pombe RAD18 and rpgL29 | |
| | genes and other members of the SMC | |
| | superfamily | |
| YOL041c | weak similarity to M. sativa NUM1, hnRNP | NOP12 |
| | protein from C. tentans and D. melanogaster, | |
| | murine/bovine p | |
| YOL045w | similarity to ser/thr protein kinase | |
| YOL046c | questionable ORF | |
| YOL054w | weak similarity to transcription factors | |
| YOL063c | hypothetical protein | |
| YOL075c | similarity to A. gambiae ATP-binding-cassette | |
| | protein | |
| YOL077c | strong similarity to C. elegans K12H4.3 protein | BRX1 |
| YOL078w | similarity to stress activated MAP kinase | |
| | interacting protein S. pombe | |
| YOL082w | similarity to YOL083w | CVT19 |
| YOL083w | similarity to YOL082w | |
| YOL084w | similarity to A. thaliana hyp1 protein | PHM7 |
| YOL087c | similarity to S. pombe hypothetical protein | |
| YOL100w | similarity to ser/thr protein kinases | PKH2 |
| YOL101c | similarity to YOL002c and YDR492w | |
| YOL111c | weak similarity to human ubiquitin-like | |
| | protein GDX | |
| YOL114c | similarity to human DS-1 protein | |
| YOL117w | weak similarity to human sodium channel alpha | |
| | chain HBA | |
| YOL128c | strong similarity to protein kinase MCK1P | |
| YOL133w | similarity to Lotus RING-finger protein | HRT1 |
| YOL138c | weak similarity to hypothetical trp-asp | |
| | repeats containing protein S. pombe | |
| YOL146w | weak similarity to hypothetical protein | |
| | S. pombe | |
| YOR054w | similarity to S. fumigata Asp FII | |
| YOR001w | similarity to human nucleolar 100K | RRP6 |
| | polymyositis-scleroderma protein | |
| YOR007c | similarity to protein phosphatases | SGT2 |
| YOR009w | similarity to TIR1P and TIR2P | TIR4 |
| YOR042w | weak similarity to YDR273w | |
| YOR051c | weak similarity to nsyosin heavy chain | |
| | proteins | |
| YOR054c | similarity to SIS2P protein and C. tropicalis | |
| | hal3 protein | |
| YOR056c | weak similarity to human phosphorylation | |
| | regulatory protein HP-10 | |
| YOR066w | hypothetical protein | |
| YOR073w | hypothetical protein | |
| YOR080w | weak similarity to | DIA2 |
| | TRCDSEMBL:RNRNAHOP_1 Rattus | |
| | norvegicus roRNA for Hsp70/Hsp90 | |
| | organizing protein | |
| YOR086c | weak similarity to synaptogamines | |
| YOR093c | similarity to S. pombe hypothetical protein | |
| | SPAC22F3.04 | |
| YOR118w | similarity to PIR:T39884 hypothetical protein | |
| | SPBC21.02 S. pombe | |
| YOR129c | weak similarity to hypothetical protein | |
| | SPBC776.06e S. pombe | |
| YOR144c | weak similarity to human DNA-binding protein | EFD1 |
| | PO-GA and to bacterial H+-transporting | |
| | ATP synthases | |
| YOR145c | strong similarity to hypothtical S. pombe | |
| | protein and to hypothetical C. elegans protein | |
| YOR154w | similarity to hypothetical A. thaliana proteins | |
| | F19G10.15 and T19F06.21 | |
| YOR155c | similarity to 5′-flanking region of the Pichia | |
| | MOX gene | |
| YOR164c | similarity to conserved hypothetical protein | |
| | S. pombe | |
| YOR172w | similarity to finger protein YKL222c, | |
| | YOR162c and YLR266c | |
| YOR173w | strong similarity to YLR270w | |
| YOR177c | weak similarity to rat SCP1 protein | |
| YOR186w | hypothetical protein | |
| YOR191w | similarity to RAD5 protein | RIS1 |
| YOR203w | questionable ORF | |
| YOR206w | similarity to Brettanomyces RAD4 and to | (RAD4) |
| | S. pombe hypothetical protein | |
| YOR214c | hypothetical protein | |
| YOR215c | similarity to M. xanthus hypothetical protein | |
| YOR220w | hypothetical protein | |
| YOR226c | strong similarity to nitrogen fixation proteins | ISU2 |
| YOR227w | similarity to microtubule-interacting protein | |
| | MHP1P | |
| YOR256c | strong similarity to secretory protein SSP134P | |
| YOR267c | similarity to ser/thr protein kinases | |
| YOR269w | similarity to human LIS-1 protein | PAC1 |
| YOR283w | weak similarity to phosphoglycerate mutases | |
| YOR285w | similarity to D. melanogaster heat shock | |
| | protein 67B2 | |
| YOR296w | similarity to hypothetical S. pombe protein | |
| YOR304c-a | similarity to mouse apolipoprotein A-IV | |
| | precursor | |
| YOR304w | strong similarity to Drosophila ISW1 and | ISW2 |
| | human SNF2P homolog | |
| YOR322c | similarity to hypothetical S. pombe protein | |
| | SPAC1F12.05 | |
| YOR324c | similarity to YAL028w | |
| YOR339c | strong similarity to E2 ubiquitin-conjugating | UBC11 |
| | enzymes | |
| YOR352w | hypothetical protein | |
| YOR353c | weak similarity to adenylate cyclases | |
| YOR356w | strong similarity to human electron transfer | |
| | flavoprotein-ubiquinone oxidoreductase | |
| YOR367w | similarity to mammalian smooth muscle protein | SCP1 |
| | SM22 and chicken calponin alpha | |
| YOR371c | similarity to YAL056w | GPE1 |
| YOR378w | strong similarity to aminotriazole resistance | |
| | protein | |
| YPL004c | strong similarity to YGR086c | |
| YPL009c | similarity to M. jannaschii hypothetical | |
| | protein | |
| YPL012w | hypothetical protein | |
| YPL013c | strong similarity to N. crassa mitochondrial | |
| | ribosomal protein S24 | |
| YPL019c | strong similarity to YFL004w, similarity to | VTC3 |
| | YJL012c | |
| YPL032c | strong similarityto PAM1P | SVL3 |
| YPL034w | questionable ORF | |
| YPL055c | hypothetical protein | |
| YPL067c | hypothetical protein | |
| YPL068c | hypothetical protein | |
| YPL074w | similarity to VPS4P and YER047c | YTA6 |
| YPL093w | similarity to M. jannaschii GTP-binding | NOG1 |
| | protein | |
| YPL109c | similarity to aminoglycoside acetyltransferase | |
| | regulator from P. stuartii | |
| YPL110c | similarity to C. elegans hypothetical protein, | |
| | weak similarity to PHO81P | |
| YPL113c | similarity to glycerate dehydrogenases | |
| YPL126w | weak similarity to fruit fly TFIID subunit p85 | NAN1 |
| YPL133c | weak similarity to transcription factors | |
| YPL135w | strong similarity to nitrogen fixation protein | ISU1 |
| | (nifU) | |
| YPL137c | similarity to microtubule-interacting protein | |
| | MHP1P and to hypothetical protein YOR227w | |
| YPL138c | weak similarity to fruit fly polycomblike | |
| | nuclear protein | |
| YPL146c | weak similarity to myosin heavy chain proteins | |
| YPL150w | similarity to ser/thr protein kinases | |
| YPL151c | strong similarity to A. thaliana PRL1 and | PRP46 |
| | PRL2 proteins | |
| YPL156c | weak similarity to YDL010w | PRM4 |
| YPL158c | weak similarity to human nucleolin | |
| YPL166w | weak similarity to paramyosins | |
| YPL168w | weak similarity to E. coli hfpB protein | |
| YPL170w | similarity to C. elegans LIM homeohox protein | |
| YPL176c | similarity to chinese hamster transferrin | SSP134 |
| | receptor protein | |
| YPL181w | weak similarity to YKR029c | |
| YPL184c | weak similarity to PUB1P | |
| YPL191c | strong similarity to YGL082w | |
| YPL206c | weak similarity to glycerophosphoryl diester | |
| | phosphodiesterases | |
| YPL207w | similarity to hypthetical proteins from A. | |
| | fulgidus, M. thermoautotrophicum and | |
| | M. jannaschii | |
| YPL208w | similarity to YHL039w | |
| YPL216w | similarity to YGL133w | |
| YPL217c | similarity to human hypothetical protein | BMS1 |
| | KIAA0187 | |
| YPL222w | similarity to C. perfringens hypothetical protein | |
| YPL226w | similarity to translation elongation factor eEF3 | NEW1 |
| YPL236c | similarity to PRK1P, and serine/threonine | |
| | protein kinase homolog from A. thaliana | |
| YPL247c | similarity to human HAN11 protein and petunia | |
| | an11 protein | |
| YPL249c | similarity to mouse Tbc1 protein | |
| YPL253c | similarity to CIK1P | VIK1 |
| YPL258c | similarity to B. subtilis transcriptional activator | THI21 |
| | tenA, and strong similarity to hypothetical | |
| | prote | |
| YPL273w | strong similarity to YLL062c | SAM4 |
| YPR003c | similarity to sulphate transporter proteins | |
| YPR015c | similarity to transcription faetors | |
| YPR021c | similarity to human citrate transporter protein | |
| YPR022c | weak similarity to fruit fly dorsal protein | |
| | and SNF5P | |
| YPR023c | similarity to human hypothetical protein | EAF3 |
| YPR030w | similarity to YBL101c | CSR2 |
| YPR037c | similarity to ERV1P and rat ALR protein | ERV2 |
| YPR038w | questionable ORF | |
| YPR040w | similarity to C. elegans C02C2.6 protein | SDF1 |
| YPR049c | similarity to USO1P | CVT9 |
| YPR078c | hypothetical protein | |
| YPR085c | hypothetical protein | |
| YPR090w | weak similarity to hypothetical protein | |
| | SPAC25B8.08 S. pombe | |
| YPR091c | weak similarity to C. elegans LIM homeobox | |
| | protein | |
| YPR093c | weak similarity to zinc-finger proteins | |
| YPR105c | similarity to hypothetical protein SPCC338.13 | |
| | S. pombe | |
| YPR115w | similarity to probable transcription factor | |
| | ASK10P, and to YNL047c and YIL10Sc | |
| YPR117w | similarity to YLR454w | |
| YPR121w | similarity to B subtilis transcriptional activator | TH122 |
| | tenA | |
| YPR130c | questionable ORF | |
| YPR139c | weak similarity to nGAP H. sapiens | |
| | nGAP mRNA | |
| YPR143w | hypothetical protein | |
| YPR184w | similarity to human 4-alpha-glucanotransferase | GDB1 |
| | (EC 2 4.1.25)/amylo-1,6-glucosidase | |
| | (EC 3.2.1.33) | |
| YPR188c | similarity to calmodulin and calinodulin-related | MLC2 |
| | proteins | |

1. A method for identifying a protein interaction network comprising two or more bait proteins, comprising:
(a) isolating complexes comprising at least one of said two or more bait proteins and their prey proteins from a sample;
(b) separating said complexes; and
(c) determining the identity of the prey proteins in each of said complexes using mass spectrometry, thereby identifying the protein interaction network.

2. A method for identifying a protein interaction network comprising two or more bait proteins, comprising:
(a) contacting said two or more bait proteins with a sample containing potential prey proteins, wherein the bait proteins and complexes comprising at least one said bait protein(s) are capable of being separated from other proteins in the sample;
(b) separating said complexes comprising at least one said bait proteins and their prey proteins; and
(c) identifying prey proteins in the complexes using mass spectrometry, thereby identifying the protein interaction network.

3. The method of claim 1, wherein steps (a)-(c) are repeated multiple times for said sample.

4. The method of claim 1, wherein said protein interaction network comprises 20 or more bait proteins.

5. The method of claim 1, wherein said protein interaction network comprises 100 or more bait proteins.

6. The method of claim 1, wherein said protein interaction network comprises bait proteins that constitute 10% or more of the proteome encoded by a given genome.

7. The method of claim 1, wherein said protein interaction network comprises all bait proteins known to be involved in the same biochemical pathway or biological process.

8. The method of claim 1, wherein said protein interaction network comprises the same type of proteins.

9. The method of claim 8, wherein said same type of proteins is protein phosphatase.

10. The method of claim 8, wherein said same type of proteins is protein kinase.

11. The method of claim 1, wherein said bait proteins are unmodified.

12. The method of claim 1, wherein said bait proteins are fused with a heterologous polypeptide.

13. The method of claim 12, wherein said heterologous polypeptide is: GST, HA epitope, c-myc epitope, 6-His tag, FLAG tag, biotin, or MBP.

14. The method of claim 1, wherein said bait proteins are expressed in a host cell as an exogenous polypeptide.

15. The method of claim 1, wherein said bait proteins are immobilized on a carrier.

16. The method of claim 1, wherein the sample is a biological sample.

17. The method of claim 16, wherein the biological sample is extract of a cell.

18. The method of claim 17, wherein the extract is concentrated.

19. The method of claim 17, wherein said cell is a yeast cell.

20. The method of claim 17, wherein said cell is from a higher eukaryote selected from:
worm (C. elegans), insect, fish, reptile, amphibian, plant, or mammal.

21. The method of claim 17, wherein said cell is a human cell.

22. The method of claim 1, wherein formation of said complexes comprising at least one of said two or more bait proteins and their prey proteins is induced using an extracellular or intracellular factor.

23. The method of claim 1, wherein the isolation step (step (a)) is effectuated by immunoprecipitation.

24. The method of claim 1, wherein the isolation step (step (a)) is effectuated by GST-pull down assay.

25. The method of claim 1, wherein said complexes are separated by SDS-PAGE.

26. The method of claim 1, wherein said complexes are separated by chromatography, HPLC, Capillary Electrophoresis (CE), isoelectric focusing (IEF).

27. The method of claim 1, wherein said complexes are digested by protease before the separation step (step (b)).

28. The method of claim 25, wherein said complexes are separated by SDS-PAGE, and wherein said complexes are digested by in-gel protease digestion after separation.

29. The method of claim 1, wherein said mass spectrometry is tandem mass spectrometry (MS/MS).

30. The method of claim 29, wherein the MS/MS is coupled with Liquid Chromatography (LC).

31. The method of claim 29, wherein step (c) includes comparing protein sequence obtained from tandem mass spectrometry with protein sequence databases.

32. The method of claim 3 1, wherein said protein sequence databases include a combination of public database and proprietary database.

33. The method of claim 1, further comprising repeating steps (a)-(c) using proteins identified from a previous round as new bait proteins, wherein said new bait proteins are different from any bait proteins used in said previous round.

34. A database of protein interaction network(s) identified by a method of the instant invention, comprising information regarding two or more bait proteins and their interactions.

35. The database of claim 34, wherein said information includes: the identity of all bait proteins and their interacting prey proteins, the conditions under which the interactions are observed, and/or the identity of the sample from which said information is obtained.

36. The database of claim 34, wherein one or more filters are used to modify the creation of said protein interaction network database.

37. The database of claim 34, wherein the database is verified by information obtained from public or proprietary database.

38. The database of claim 34, wherein the database comprises a set of potential protein interactions and molecular complexes in a given proteome, under one or more specific conditions.

39. The database of claim 34, wherein the database comprises at least about 30% of the potential protein interactions of a given organism.

40. The database of claim 34, further comprising annotations of certain protein-protein interaction information obtained from searching available scientific literature using proprietary software.

41. The database of claim 40, wherein said annotations are dynamically updated, preferably automatically, by repeated searches performed at predetermined time intervals.

42. The database of claim 39, wherein the organism is a yeast.

43. The database of claim 42, wherein the database comprises a set of more than 4000 yeast protein interactions.

44. The database of claim 42, wherein the database comprises the complexes of Table 2, 4A, 4B, 5A, 5B, and 7.

45. A method of identifying differences in protein interaction networks comprising one or more selected bait proteins, comprising:
(a) providing a first protein interaction network identified by (i) isolating complexes comprising a selected bait protein(s) and prey proteins from a first sample; (ii) separating complexes comprising the bait protein(s) and prey proteins; and (iii) determining the identity of the prey proteins, preferably by mass spectrometry, thereby identifying the first protein interaction network;
(b) providing a second protein interaction network identified by (i) isolating complexes comprising the selected bait protein(s) and prey proteins from a second sample; (ii) separating complexes comprising the bait protein(s) and prey proteins; and (iii) determining the identity of the prey proteins, preferably by mass spectrometry, thereby identifying the second protein interaction network; and
(c) comparing the first and second protein interaction networks, thereby identifying differences in the protein interaction networks.

46. The method of claim 45, wherein the first sample is from a tumor tissue, and the second sample is from a normal tissue of the same tissue.

47. The method of claim 45, wherein the tumor tissue and the normal tissue are from the same patient.

48. The method of claim 45, wherein the first sample and the second sample are from different developmental stages of the same organism.

49. The method of claim 45, wherein the first sample is from a tissue, and the second sample is from the same tissue after a treatment.

50. The method of claim 49, wherein the tissue is a tumor tissue.

51. The method of claim 49, wherein the treatment is chemotherapy, or radiotherapy.

52. A method of assaying for changes in protein interaction networks in response to an intracellular or extracellular factor comprising:
(a) contacting two or more bait proteins with a sample containing prey proteins in the presence of an intracellular or extracellular factor, wherein the bait proteins and complexes comprising the bait proteins are capable of being separated from other proteins in the sample;
(b) separating complexes comprising bait proteins and prey proteins;
(c) identifying prey proteins in the complexes using mass spectrometry, thereby identifying the protein interaction network; and
(d) comparing the protein interaction network identified in (c) with a protein interaction network identified in the absence of the intracellular or extracellular factor.

53. A method of conducting a pharmaceutical business, comprising:
(a) identifying a protein interaction network of one or more known bait proteins from a sample using a method of the invention wherein said bait protein is a potential drug target;
(b) identifying, among prey proteins that interact with said bait proteins in the protein interaction network, new potential drug targets; and
(c) licensing, to a third party, the rights for further drug development of inhibitors or activators of the drug target.

54. A method of conducting a pharmaceutical business, comprising:
(a) identifying a protein interaction network of one or more known bait proteins from a biological sample using a method of the invention, wherein said bait protein is a potential drug target;
(b) identifying, among prey proteins that interact with said bait proteins in the protein interaction network, new potential drug targets;
(c) identifying compounds that modulate activity of said new potential drug targets;
(d) conducting therapeutic profiling of compounds identified in step (c), or further analogs thereof, for efficacy and toxicity in animals; and,
(e) formulating a pharmaceutical preparation including one or more compounds identified in step (d) as having an acceptable therapeutic profile.

55. The business method of claim 54, further comprising an additional step of establishing a distribution system for distributing the pharmaceutical preparation for sale.

56. The business method of claim 54, further including establishing a sales group for marketing the pharmaceutical preparation.

57. A method for constructing a protein interaction network map for a proteome comprising:
(a) identifying a protein interaction network according to claim 1; and
(b) displaying the network as a linkage map.

58. An integrated modular system for performing the method of claim 1, the system comprising one or more of:
(a) a module for retrieving recombinant clones encoding bait proteins;
(b) an automated immunoprecipitation module for purification of complexes comprising bait and prey proteins;
(c) an analysis module for further purifying the proteins from (b) or preparing fragments of said proteins that are suitable for mass spectrometry;
(d) a mass spectrometer module for automated analysis of fragments from (c);
(e) a computer module comprising an integration software for communication among the modules of the system and integrating operations; and
(f) a module for integrating the operation of one or more of (a)-(d).
